SEQUENCE LISTING (196 sequences)

SEQ ID NO: 1 - DNA, 51 bp, Artificial Sequence
  Chemically Synthesized
  misc_feature (2)..(2): n is Deoxyuridine
  misc_feature (10)..(10): n is Deoxyuridine
  misc_feature (14)..(14): n is Deoxyuridine
  misc_feature (18)..(34): n is a, c, g, or t
gnggagaggntctnacannnnnnnnnnnnnnnnntgtgagagcctctccgc

SEQ ID NO: 2 - DNA, 53 bp, Artificial Sequence
  Chemically Synthesized
  misc_feature (2)..(2): n is Deoxyuridine
  misc_feature (10)..(10): n is Deoxyuridine
  misc_feature (14)..(14): n is Deoxyuridine
  misc_feature (18)..(36): n is a, c, g, or t
gnggagaggntctnacannnnnnnnnnnnnnnnnnntgtgagagcctctccgc

SEQ ID NO: 3 - DNA, 55 bp, Artificial Sequence
  Chemically Synthesized
  misc_feature (2)..(2): n is Deoxyuridine
  misc_feature (10)..(10): n is Deoxyuridine
  misc_feature (14)..(14): n is Deoxyuridine
  misc_feature (18)..(38): n is a, c, g, or t
gnggagaggntctnacannnnnnnnnnnnnnnnnnnnntgtgagagcctctccgc

SEQ ID NO: 4 - DNA, 57 bp, Artificial Sequence
  Chemically Synthesized
  misc_feature (2)..(2): n is Deoxyuridine
  misc_feature (10)..(10): n is Deoxyuridine
  misc_feature (14)..(14): n is Deoxyuridine
  misc_feature (18)..(40): n is a, c, g, or t
gnggagaggntctnacannnnnnnnnnnnnnnnnnnnnnntgtgagagcctctccgc

SEQ ID NO: 5 - DNA, 51 bp, Artificial Sequence
  Chemically Synthesized
caagcagaagacggcatacgagctcttccgatctgcggagaggctctcaca

SEQ ID NO: 6 - DNA, 81 bp, Artificial Sequence
  Chemically Synthesized
aatgatacggcgaccaccgagatctacactctttccctacacgacgctcttccgatcttctactgtggagaggttcttaca

SEQ ID NO: 7 - DNA, 41 bp, Artificial Sequence
  Chemically Synthesized
gaggctctcacatcgccctcccttgacggtgtgagagcctc

SEQ ID NO: 8 - DNA, 41 bp, Artificial Sequence
  Chemically Synthesized
gaggctctcacatcacccaccttcaatggtgtgagagcctc

SEQ ID NO: 9 - DNA, 41 bp, Artificial Sequence
  Chemically Synthesized
gaggctctcacactttgccttgcggaccttgtgagagcctc

SEQ ID NO: 10 - DNA, 41 bp, Artificial Sequence
  Chemically Synthesized
gaggctctcacatcattaggcgtaattggtgtgagagcctc

SEQ ID NO: 11 - DNA, 41 bp, Artificial Sequence
  Chemically Synthesized
gaggctctcacatctatcggctttacaggtgtgagagcctc

SEQ ID NO: 12 - DNA, 41 bp, Artificial Sequence
  Chemically Synthesized
gaggctctcacataaaaggggcagggtggtgtgagagcctc

SEQ ID NO: 13 - DNA, 41 bp, Artificial Sequence
  Chemically Synthesized
gaggctctcacattggtcccctttatcggtgtgagagcctc

SEQ ID NO: 14 - DNA, 41 bp, Artificial Sequence
  Chemically Synthesized
gaggctctcacattggggtgtacttacggtgtgagagcctc

SEQ ID NO: 15 - DNA, 41 bp, Artificial Sequence
  Chemically Synthesized
gaggctctcacattaggcggcacctctagtgtgagagcctc

SEQ ID NO: 16 - DNA, 41 bp, Artificial Sequence; identical to SEQ ID NO: 14
  Chemically Synthesized

SEQ ID NO: 17 - DNA, 41 bp, Artificial Sequence
  Chemically Synthesized
gaggctctcacattcactatacccttgattgtgagagcctc

SEQ ID NO: 18 - DNA, 41 bp, Artificial Sequence
  Chemically Synthesized
gaggctctcacatgtcctaacctctccggtgtgagagcctc

SEQ ID NO: 19 - DNA, 41 bp, Artificial Sequence
  Chemically Synthesized
gaggctctcacaactcagccctcccagggtgtgagagcctc

SEQ ID NO: 20 - DNA, 41 bp, Artificial Sequence
  Chemically Synthesized
gaggctctcacactatcggacgcggtacttgtgagagcctc

SEQ ID NO: 21 - DNA, 47 bp, Artificial Sequence
  Chemically Synthesized
gaggctctcacaggaatccaagctcggcctcccggtgtgagagcctc

SEQ ID NO: 22 - DNA, 47 bp, Artificial Sequence
  Chemically Synthesized
gaggctctcacacggttacggtcacccaagcgcattgtgagagcctc

SEQ ID NO: 23 - DNA, 47 bp, Artificial Sequence
  Chemically Synthesized
gaggctctcacatccacccttccgcgatgacatggtgtgagagcctc

SEQ ID NO: 24 - DNA, 47 bp, Artificial Sequence
  Chemically Synthesized
gaggctctcacatcactgatcacagctctttttggtgtgagagcctc

SEQ ID NO: 25 - DNA, 47 bp, Artificial Sequence
  Chemically Synthesized
gaggctctcacatcgcaaaaagggtcagaattcggtgtgagagcctc

SEQ ID NO: 26 - DNA, 47 bp, Artificial Sequence
  Chemically Synthesized
gaggctctcacatcgccccattccctgttgctcggtgtgagagcctc

SEQ ID NO: 27 - DNA, 47 bp, Artificial Sequence
  Chemically Synthesized
gaggctctcacatcgcgcctttccccagctttcggtgtgagagcctc

SEQ ID NO: 28 - DNA, 47 bp, Artificial Sequence
  Chemically Synthesized
gaggctctcacatcgatgcctcctttactatacggtgtgagagcctc

SEQ ID NO: 29 - DNA, 47 bp, Artificial Sequence
  Chemically Synthesized
gaggctctcacatcgttccaccctttctgtttcggtgtgagagcctc

SEQ ID NO: 30 - DNA, 47 bp, Artificial Sequence
  Chemically Synthesized
gaggctctcacatcgtggtcaggttgatttgctggtgtgagagcctc

SEQ ID NO: 31 - DNA, 47 bp, Artificial Sequence
  Chemically Synthesized
gaggctctcacatcggtgtggcaggtttattacggtgtgagagcctc

SEQ ID NO: 32 - DNA, 47 bp, Artificial Sequence
  Chemically Synthesized
gaggctctcacatcggatacacaccaactgcttggtgtgagagcctc

SEQ ID NO: 33 - DNA, 47 bp, Artificial Sequence
  Chemically Synthesized
gaggctctcacatcggacgacctaaggcaaaacggtgtgagagcctc

SEQ ID NO: 34 - DNA, 47 bp, Artificial Sequence
  Chemically Synthesized
gaggctctcacacactccttcatactccctcggcctgtgagagcctc

SEQ ID NO: 35 - DNA, 47 bp, Artificial Sequence
  Chemically Synthesized
gaggctctcacaactatgcgctggcacctcttgtctgtgagagcctc

SEQ ID NO: 36 - DNA, 47 bp, Artificial Sequence
  Chemically Synthesized
gaggctctcacaacgctccctcccaagtattatggtgtgagagcctc

SEQ ID NO: 37 - DNA, 47 bp, Artificial Sequence
  Chemically Synthesized
gaggctctcacatcttcggctccctcctctcagactgtgagagcctc

SEQ ID NO: 38 - DNA, 47 bp, Artificial Sequence
  Chemically Synthesized
gaggctctcacatcgttctttggtgcggtgaatggtgtgagagcctc

SEQ ID NO: 39 - DNA, 47 bp, Artificial Sequence
  Chemically Synthesized
gaggctctcacatcgggggcgctctttaatattggtgtgagagcctc

SEQ ID NO: 40 - DNA, 47 bp, Artificial Sequence
  Chemically Synthesized
gaggctctcacataagtgtgatcgagccctcctggtgtgagagcctc

SEQ ID NO: 41 - DNA, 47 bp, Artificial Sequence
  Chemically Synthesized
gaggctctcacatttactctcgccatcgatcacggtgtgagagcctc

SEQ ID NO: 42 - DNA, 47 bp, Artificial Sequence
  Chemically Synthesized
gaggctctcacaacgcgcctcccgtccgaattcggtgtgagagcctc

SEQ ID NO: 43 - DNA, 46 bp, Artificial Sequence
  Chemically Synthesized
gaggctctcacactgtcgcgcctctccggatatggtgtgagagcct

SEQ ID NO: 44 - DNA, 46 bp, Artificial Sequence
  Chemically Synthesized
gaggctctcacatgcgcagtcccctcacgttaccttgtgagagcct

SEQ ID NO: 45 - DNA, 47 bp, Artificial Sequence
  Chemically Synthesized
gaggctctcacaaccacgttcccggcatgtcattatgtgagagcctc

SEQ ID NO: 46 - DNA, 45 bp, Artificial Sequence
  Chemically Synthesized
gaggctctcacatcgcggtagtccctttttcggtgtgagagcctc

SEQ ID NO: 47 - DNA, 45 bp, Artificial Sequence
  Chemically Synthesized
gaggctctcacacgttcgctgttcgtggtaatatgtgagagcctc

SEQ ID NO: 48 - DNA, 45 bp, Artificial Sequence
  Chemically Synthesized
gaggctctcacattgtgcgatccctttatacggtgtgagagcctc

SEQ ID NO: 49 - DNA, 45 bp, Artificial Sequence
  Chemically Synthesized
gaggctctcacatatgccagctttccatcacggtgtgagagcctc

SEQ ID NO: 50 - DNA, 43 bp, Artificial Sequence
  Chemically Synthesized
gaggctctcacaactccgcgccgacccattatgtgagagcctc

SEQ ID NO: 51 - DNA, 43 bp, Artificial Sequence
  Chemically Synthesized
gaggctctcacacgccggattcccctgtatttgtgagagcctc

SEQ ID NO: 52 - DNA, 43 bp, Artificial Sequence
  Chemically Synthesized
gaggctctcacatctggggcggtcattaaggtgtgagagcctc

SEQ ID NO: 53 - DNA, 43 bp, Artificial Sequence
  Chemically Synthesized
gaggctctcacatcggtccccctttaaacggtgtgagagcctc

SEQ ID NO: 54 - DNA, 43 bp, Artificial Sequence
  Chemically Synthesized
gaggctctcacatcggcctctccttgtttggtgtgagagcctc

SEQ ID NO: 55 - DNA, 43 bp, Artificial Sequence
  Chemically Synthesized
gaggctctcacatcgccctctcggcactcggtgtgagagcctc

SEQ ID NO: 56 - DNA, 43 bp, Artificial Sequence
  Chemically Synthesized
gaggctctcacatcgcacaggtttagtacggtgtgagagcctc

SEQ ID NO: 57 - DNA, 43 bp, Artificial Sequence
  Chemically Synthesized
gaggctctcacatcaggctcctccttattggtgtgagagcctc

SEQ ID NO: 58 - DNA, 43 bp, Artificial Sequence
  Chemically Synthesized
gaggctctcacatctgttgcctctccggaactgtgagagcctc

SEQ ID NO: 59 - DNA, 43 bp, Artificial Sequence
  Chemically Synthesized
gaggctctcacaatcatactccccgctttggtgtgagagcctc

SEQ ID NO: 60 - DNA, 43 bp, Artificial Sequence
  Chemically Synthesized
gaggctctcacaggggttccgtagggagtggtgtgagagcctc

SEQ ID NO: 61 - DNA, 43 bp, Artificial Sequence
  Chemically Synthesized
gaggctctcacattggcacatggcgttacggtgtgagagcctc

SEQ ID NO: 62 - DNA, 41 bp, Artificial Sequence
  Chemically Synthesized
gaggctctcacattggggcgtgctgacggtgtgagagcctc

SEQ ID NO: 63 - DNA, 41 bp, Artificial Sequence
  Chemically Synthesized
gaggctctcacattgcactccttcatcggtgtgagagcctc

SEQ ID NO: 64 - DNA, 41 bp, Artificial Sequence
  Chemically Synthesized
gaggctctcacatcgggctcctttaacggtgtgagagcctc

SEQ ID NO: 65 - DNA, 41 bp, Artificial Sequence
  Chemically Synthesized
gaggctctcacactttgctggggcggacttgtgagagcctc

SEQ ID NO: 66 - DNA, 41 bp, Artificial Sequence
  Chemically Synthesized
gaggctctcacatcgggctcctttatcggtgtgagagcctc

SEQ ID NO: 67 - DNA, 717 bp, Aequorea Victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 68 - RNA, 104 nt, Artificial Sequence
  Chemically Synthesized
ggccucgaacuucaccucggcgguuuuagagcuagaaauagcaaguuaaaauaaggcuaguccguuaucaacuugaaaaguggcaccgagucggugcuuuuuuu

SEQ ID NO: 69 - RNA, 105 nt, Artificial Sequence
  Chemically Synthesized
gguuuugcaguuuaucaggaugguuuuagagcuagaaauagcaaguuaaaauaaggcuaguccguuaucaacuugaaaaaguggcaccgagucggugcuuuuuuu

SEQ ID NO: 70 - DNA, 41 bp, Artificial Sequence
  Chemically Synthesized
gaggctctcacacgcgacgaagctggacatgtgagagcctc

SEQ ID NO: 71 - DNA, 41 bp, Artificial Sequence
  Chemically Synthesized
gaggctctcacaggagcgacgggatctcatgtgagagcctc

SEQ ID NO: 72 - DNA, 69 bp, Artificial Sequence
  Chemically Synthesized
gaggctctcacaggtccggtacggcacggtcgcgaagcgaggtctggggtggggaggtgtgagagcctc

SEQ ID NO: 73 - DNA, 41 bp, Artificial Sequence
  Chemically Synthesized
gaggctctcacatcgcgcctttctttcggtgtgagagcctc

SEQ ID NO: 74 - DNA, 37 bp, Artificial Sequence
  Chemically Synthesized
gaggctctcacatcgcgcctttcggtgtgagagcctc

SEQ ID NO: 75 - DNA, 36 bp, Artificial Sequence
  Chemically Synthesized
gaggctctcacatcgcgccttcggtgtgagagcctc

SEQ ID NO: 76 - DNA, 40 bp, Artificial Sequence
  Chemically Synthesized
gaggctctcacatcgcgccttttttcggtgtgagagcctc

SEQ ID NO: 77 - DNA, 39 bp, Artificial Sequence
  Chemically Synthesized
gaggctctcacatcgcgcctttttcggtgtgagagcctc

SEQ ID NO: 78 - DNA, 38 bp, Artificial Sequence
  Chemically Synthesized
gaggctctcacatcgcgccttttcggtgtgagagcctc

SEQ ID NO: 79 - DNA, 34 bp, Artificial Sequence
  Chemically Synthesized
gaggctctcacatcggtaacggtgtgagagcctc

SEQ ID NO: 80 - DNA, 43 bp, Artificial Sequence; identical to SEQ ID NO: 50
  Chemically Synthesized

SEQ ID NO: 81 - DNA, 43 bp, Artificial Sequence
  Chemically Synthesized
gaggctctcacatgggtacgcgcctttgtggtgtgagagcctc

SEQ ID NO: 82 - DNA, 43 bp, Artificial Sequence
  Chemically Synthesized
gaggctctcacatcgcgcccctcatcatcggtgtgagagcctc

SEQ ID NO: 83 - DNA, 43 bp, Artificial Sequence
  Chemically Synthesized
gaggctctcacattgccctctttcaatacggtgtgagagcctc

SEQ ID NO: 84 - DNA, 47 bp, Artificial Sequence
  Chemically Synthesized
gaggctctcacatcgcgcctccctgtaaaattcggtgtgagagcctc

SEQ ID NO: 85 - DNA, 47 bp, Artificial Sequence
  Chemically Synthesized
gaggctctcacatcgcgcctctccgcaacatacggtgtgagagcctc

SEQ ID NO: 86 - DNA, 47 bp, Artificial Sequence
  Chemically Synthesized
gaggctctcacatcgtgacctactcgcgccgtatgtgtgagagcctc

SEQ ID NO: 87 - DNA, 47 bp, Artificial Sequence
  Chemically Synthesized
gaggctctcacactgtcgcgcctctccggatatggtgtgagagcctc

SEQ ID NO: 88 - DNA, 47 bp, Artificial Sequence
  Chemically Synthesized
gaggctctcacaccctccacacgcgcctggtcccatgtgagagcctc

SEQ ID NO: 89 - DNA, 47 bp, Artificial Sequence
  Chemically Synthesized
gaggctctcacatctcgcctttccccagctttcggtgtgagagcctc

SEQ ID NO: 90 - DNA, 47 bp, Artificial Sequence
  Chemically Synthesized
gaggctctcacaacgcgcctcccgtccgaattaggtgtgagagcctc

SEQ ID NO: 91 - DNA, 37 bp, Artificial Sequence
  Chemically Synthesized
gaggctctcacatcgggcctttcggtgtgagagcctc

SEQ ID NO: 92 - DNA, 37 bp, Artificial Sequence
  Chemically Synthesized
gaggctctcacatcgcccctttcggtgtgagagcctc

SEQ ID NO: 93 - DNA, 37 bp, Artificial Sequence
  Chemically Synthesized
gaggctctcacatcgcggctttcggtgtgagagcctc

SEQ ID NO: 94 - DNA, 37 bp, Artificial Sequence
  Chemically Synthesized
gaggctctcacatcgcgcgtttcggtgtgagagcctc

SEQ ID NO: 95 - DNA, 37 bp, Artificial Sequence
  Chemically Synthesized
gaggctctcacatcgcgccattcggtgtgagagcctc

SEQ ID NO: 96 - DNA, 37 bp, Artificial Sequence
  Chemically Synthesized
gaggctctcacatcgcgcctatcggtgtgagagcctc

SEQ ID NO: 97 - DNA, 37 bp, Artificial Sequence
  Chemically Synthesized
gaggctctcacatcgcgccttacggtgtgagagcctc

SEQ ID NO: 98 - DNA, 35 bp, Artificial Sequence
  Chemically Synthesized
aggctctcacatcgcgcctttcggtgtgagagcct

SEQ ID NO: 99 - DNA, 33 bp, Artificial Sequence
  Chemically Synthesized
ggctctcacatcgcgcctttcggtgtgagagcc

SEQ ID NO: 100 - DNA, 31 bp, Artificial Sequence
  Chemically Synthesized
gctctcacatcgcgcctttcggtgtgagagc

SEQ ID NO: 101 - DNA, 29 bp, Artificial Sequence
  Chemically Synthesized
ctctcacatcgcgcctttcggtgtgagag

SEQ ID NO: 102 - DNA, 25 bp, Artificial Sequence
  Chemically Synthesized
ctcacatcgcgcctttcggtgtgag

SEQ ID NO: 103 - DNA, 19 bp, Artificial Sequence
  Chemically Synthesized
acatcgcgcctttcggtgt

SEQ ID NO: 104 - DNA, 13 bp, Artificial Sequence
  Chemically Synthesized
tcgcgcctttcgg

SEQ ID NO: 105 - DNA, 37 bp, Artificial Sequence
  Chemically Synthesized
ctggctctcacatcgcgcctttcggtgtgagagccag

SEQ ID NO: 106 - DNA, 37 bp, Artificial Sequence
  Chemically Synthesized
gaccctctcacatcgcgcctttcggtgtgagagggtc

SEQ ID NO: 107 - DNA, 37 bp, Artificial Sequence
  Chemically Synthesized
gagggactcacatcgcgcctttcggtgtgagtccctc

SEQ ID NO: 108 - DNA, 37 bp, Artificial Sequence
  Chemically Synthesized
gaggctgacacatcgcgcctttcggtgtgtcagcctc

SEQ ID NO: 109 - DNA, 37 bp, Artificial Sequence
  Chemically Synthesized
gaggctctgtcatcgcgcctttcggtgacagagcctc

SEQ ID NO: 110 - DNA, 37 bp, Artificial Sequence
  Chemically Synthesized
gaggctctcagttcgcgcctttcggactgagagcctc

SEQ ID NO: 111 - DNA, 37 bp, Artificial Sequence
  Chemically Synthesized
gaggctctcacatgccgcctttgcgtgtgagagcctc

SEQ ID NO: 112 - DNA, 37 bp, Artificial Sequence
  Chemically Synthesized
gaggctctcacaacgcgcctttcggtgtgagagcctc

SEQ ID NO: 113 - DNA, 37 bp, Artificial Sequence
  Chemically Synthesized
gaggctctcacagcgcgcctttcgttgtgagagcctc

SEQ ID NO: 114 - DNA, 37 bp, Artificial Sequence
  Chemically Synthesized
gaggctctcacaccgcgcctttcggtgtgagagcctc

SEQ ID NO: 115 - DNA, 37 bp, Artificial Sequence
  Chemically Synthesized
gaggctctcacatcgcgccttttggtgtgagagcctc

SEQ ID NO: 116 - DNA, 37 bp, Artificial Sequence
  Chemically Synthesized
gaggctctcacatcacgccttttggtgtgagagcctc

SEQ ID NO: 117 - DNA, 37 bp, Artificial Sequence
  Chemically Synthesized
gaggctctcacatcccgcctttgggtgtgagagcctc

SEQ ID NO: 118 - DNA, 37 bp, Artificial Sequence
  Chemically Synthesized
gaggctctcacatcacgcctttcggtgtgagagcctc

SEQ ID NO: 119 - DNA, 37 bp, Artificial Sequence
  Chemically Synthesized
gaggctctcacatggcgcctttccgtgtgagagcctc

SEQ ID NO: 120 - DNA, 37 bp, Artificial Sequence
  Chemically Synthesized
gaggctctcacattgcgcctttcggtgtgagagcctc

SEQ ID NO: 121 - DNA, 37 bp, Artificial Sequence
  Chemically Synthesized
gaggctctcacatagcgcctttcggtgtgagagcctc

SEQ ID NO: 122 - DNA, 38 bp, Artificial Sequence
  Chemically Synthesized
gaggctctcacatccacgccttttggtgtgagagcctc

SEQ ID NO: 123 - DNA, 37 bp, Artificial Sequence; identical to SEQ ID NO: 74
  Chemically Synthesized
  modified_base (1)..(3)
  modified_base (34)..(36): phosphorotioate nucleotide

SEQ ID NO: 124 - DNA, 37 bp, Artificial Sequence; identical to SEQ ID NO: 74
  Chemically Synthesized
  modified_base (4)..(6): phosphorotioate nucleotide
  modified_base (31)..(33): phosphorotioate nucleotide

SEQ ID NO: 125 - DNA, 37 bp, Artificial Sequence; identical to SEQ ID NO: 74
  Chemically Synthesized
  modified_base (7)..(9): phosphorotioate nucleotide
  modified_base (28)..(30): phosphorotioate nucleotide

SEQ ID NO: 126 - DNA, 37 bp, Artificial Sequence; identical to SEQ ID NO: 74
  Chemically Synthesized
  modified_base (10)..(12): phosphorotioate nucleotide
  modified_base (25)..(27): phosphorotioate nucleotide

SEQ ID NO: 127 - DNA, 37 bp, Artificial Sequence; identical to SEQ ID NO: 74
  Chemically Synthesized
  modified_base (13)..(15): phosphorotioate nucleotide
  modified_base (22)..(24): phosphorotioate nucleotide

SEQ ID NO: 128 - DNA, 37 bp, Artificial Sequence; identical to SEQ ID NO: 74
  Chemically Synthesized
  modified_base (16)..(22): phosphorotioate nucleotide

SEQ ID NO: 129 - DNA, 37 bp, Artificial Sequence; identical to SEQ ID NO: 74
  Chemically Synthesized
  modified_base (1)..(37): phosphorotioate nucleotide

SEQ ID NO: 130 - DNA, 29 bp, Artificial Sequence
  Chemically Synthesized
attgaccgctgtgtgacgcaacactcaat

SEQ ID NO: 131 - DNA, 38 bp, Artificial Sequence
  Chemically Synthesized
cggtgtgagagcctcgagggaggctctcacatcgcgcc

SEQ ID NO: 132 - RNA, 43 nt, Artificial Sequence
  Chemically Synthesized
ggccucgaacuucaccucggcgguuuuagagcuaugcuguuug

SEQ ID NO: 133 - RNA, 41 nt, Artificial Sequence
  Chemically Synthesized
caacuacaagacccgcgccgguuuuagagcuaugcuguuug

SEQ ID NO: 134 - RNA, 41 nt, Artificial Sequence
  Chemically Synthesized
cgaugcccuucagcucgaugguuuuagagcuaugcuguuug

SEQ ID NO: 135 - RNA, 41 nt, Artificial Sequence
  Chemically Synthesized
caugccgagagugaucccggguuuuagagcuaugcuguuug

SEQ ID NO: 136 - DNA, 22 bp, Artificial Sequence
  Chemically Synthesized
cgccgaggtgaagttcgaggcc

SEQ ID NO: 137 - RNA, 22 nt, Artificial Sequence
  Chemically Synthesized
cgccgaggugaaguucgaggcc

SEQ ID NO: 138 - DNA, 38 bp, Artificial Sequence
  Chemically Synthesized
cggtgtgagagcctcgagggaggctctcacatcgcggc

SEQ ID NO: 139 - DNA, 38 bp, Artificial Sequence
  Chemically Synthesized
cggtgtgagagcctcgagggaggctctcacatcgcatc

SEQ ID NO: 140 - DNA, 37 bp, Artificial Sequence; identical to SEQ ID NO: 93
  Chemically Synthesized

SEQ ID NO: 141 - DNA, 37 bp, Artificial Sequence
  Chemically Synthesized
gaggctctcacatcgcatctttcggtgtgagagcctc

SEQ ID NO: 142 - DNA, 47 bp, Artificial Sequence
  Chemically Synthesized
gaggctctcacatcgcgccaaaaaaaaaaaaacggtgtgagagcctc

SEQ ID NO: 143 - DNA, 47 bp, Artificial Sequence
  Chemically Synthesized
gaggctctcacatcgcggcaaaaaaaaaaaaacggtgtgagagcctc

SEQ ID NO: 144 - DNA, 47 bp, Artificial Sequence
  Chemically Synthesized
gaggctctcacatcgcatcaaaaaaaaaaaaacggtgtgagagcctc

SEQ ID NO: 145 - DNA, 20 bp, Artificial Sequence
  Chemically Synthesized
cggcgcgggtcttgtagttg

SEQ ID NO: 146 - DNA, 20 bp, Artificial Sequence
  Chemically Synthesized
catcgagctgaagggcatcg

SEQ ID NO: 147 - DNA, 34 bp, Artificial Sequence
  Chemically Synthesized
cggtgtgagagcctcgagggaggctctcacatcg

SEQ ID NO: 148 - DNA, 36 bp, Artificial Sequence
  Chemically Synthesized
cggtgtgagagcctcgaaagaggctctcacatcgcg

SEQ ID NO: 149 - DNA, 37 bp, Artificial Sequence
  Chemically Synthesized
cggtgtgagagcctcgaaagaggctctcacatcgcgc

SEQ ID NO: 150 - DNA, 39 bp, Artificial Sequence
  Chemically Synthesized
cggtgtgagagcctcgaaagaggctctcacatcgcgccg

SEQ ID NO: 151 - DNA, 40 bp, Artificial Sequence
  Chemically Synthesized
cggtgtgagagcctcgaaagaggctctcacatcgcgccga

SEQ ID NO: 152 - DNA, 42 bp, Artificial Sequence
  Chemically Synthesized
ggcgcggtgtgagagcctcgaaagaggctctcacatcgcgcc

SEQ ID NO: 153 - DNA, 37 bp, Artificial Sequence
  Chemically Synthesized
gaggctctcacatcgcgccgaggtgaagttcgaggcc

SEQ ID NO: 154 - DNA, 38 bp, Artificial Sequence
  Chemically Synthesized
ggcctcgaacttcacctcggcgcgatgtgagagcctca

SEQ ID NO: 155 - DNA, 39 bp, Artificial Sequence
  Chemically Synthesized
tcggtgtgagagcctcgaaagaggctctcacatcgcgcc

SEQ ID NO: 156 - DNA, 40 bp, Artificial Sequence
  Chemically Synthesized
ttcggtgtgagagcctcgaaagaggctctcacatcgcgcc

SEQ ID NO: 157 - DNA, 41 bp, Artificial Sequence
  Chemically Synthesized
tttcggtgtgagagcctcgaaagaggctctcacatcgcgcc

SEQ ID NO: 158 - DNA, 26 bp, Artificial Sequence
  Chemically Synthesized
cggtgtgaggaaactcacatcgcgcc

SEQ ID NO: 159 - DNA, 3630 bp, Homo sapiens
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcaactcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagca

SEQ ID NO: 160 - RNA, 41 nt, Artificial Sequence
  Chemically Synthesized
aucauaauuccucugcacauguuuuagagcuaugcuguuug

SEQ ID NO: 161 - RNA, 41 nt, Artificial Sequence
  Chemically Synthesized
ccacuguguugagggcaaugguuuuagagcuaugcuguuug

SEQ ID NO: 162 - DNA, 38 bp, Artificial Sequence
  Chemically Synthesized
cggtgtgagagcctcgaaagaggctctcacatcgatgt

SEQ ID NO: 163 - DNA, 38 bp, Artificial Sequence
  Chemically Synthesized
cggtgtgagagcctcgaaagaggctctcacatcgcatt

SEQ ID NO: 164 - DNA, 39 bp, Artificial Sequence
  Chemically Synthesized
cggtgtgagagcctcgaaagaggctctcacatcgatgtg

SEQ ID NO: 165 - DNA, 40 bp, Artificial Sequence
  Chemically Synthesized
cggtgtgagagcctcgaaagaggctctcacatcgatgtgc

SEQ ID NO: 166 - DNA, 39 bp, Artificial Sequence
  Chemically Synthesized
cggtgtgagagcctcgaaagaggctctcacatcgcattg

SEQ ID NO: 167 - DNA, 40 bp, Artificial Sequence
  Chemically Synthesized
cggtgtgagagcctcgaaagaggctctcacatcgcattgc

SEQ ID NO: 168 - DNA, 20 bp, Artificial Sequence
  Chemically Synthesized
atgtgcagaggaattatgat

SEQ ID NO: 169 - DNA, 20 bp, Artificial Sequence
  Chemically Synthesized
cattgccctcaacacagtgg

SEQ ID NO: 170 - DNA, 23 bp, Artificial Sequence
  Chemically Synthesized
gtgcaccaactgaagtacaccgg

SEQ ID NO: 171 - RNA, 41 nt, Artificial Sequence
  Chemically Synthesized
gugcaccaacugaaguacacguuuuagagcuaugcuguuug

SEQ ID NO: 172 - DNA, 38 bp, Artificial Sequence
  Chemically Synthesized
cggtgtgagagcctcgaaagaggctctcacatcggtgt

SEQ ID NO: 173 - RNA, 20 nt, Artificial Sequence
  Chemically Synthesized
cgucgccguccagcucgacc

SEQ ID NO: 174 - DNA, 38 bp, Artificial Sequence
  Chemically Synthesized
gacgcaaagtgagagcctcgaaagaggctctcactttg

SEQ ID NO: 175 - DNA, 38 bp, Artificial Sequence
  Chemically Synthesized
gacgcgaagtgagagcctcgaaagaggctctcactttg

SEQ ID NO: 176 - DNA, 38 bp, Artificial Sequence
  Chemically Synthesized
gacgcagagtgagagcctcgaaagaggctctcactttg

SEQ ID NO: 177 - DNA, 38 bp, Artificial Sequence
  Chemically Synthesized
gacgcaaggtgagagcctcgaaagaggctctcactttg

SEQ ID NO: 178 - RNA, 20 nt, Artificial Sequence
  Chemically Synthesized
gugccaccugcgugaagaag

SEQ ID NO: 179 - RNA, 20 nt, Artificial Sequence
  Chemically Synthesized
cgccagaccaggcagucgcu

SEQ ID NO: 180 - RNA, 20 nt, Artificial Sequence
  Chemically Synthesized
uggcaguucuccucuccugc

SEQ ID NO: 181 - DNA, 38 bp, Artificial Sequence
  Chemically Synthesized
gcaccaaagtgagagcctcgaaagaggctctcactttg

SEQ ID NO: 182 - DNA, 38 bp, Artificial Sequence
  Chemically Synthesized
gccgcaaagtgagagcctcgaaagaggctctcactttg

SEQ ID NO: 183 - DNA, 38 bp, Artificial Sequence
  Chemically Synthesized
gccacaaagtgagagcctcgaaagaggctctcactttg

SEQ ID NO: 184 - DNA, 837 bp, Artificial Sequence
  Chemically synthesized
atggcgtcttcttctcatttcacaccgaagcagagtttttcggattcccgagtagcagatgaccatgacaagtagcggcaggaccagccccaagatgacttggagataactactaagaagaaaacggtgagcaagggcgaggaggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaag

SEQ ID NO: 185 - RNA, 41 nt, Artificial Sequence
  Chemically Synthesized
cagaugaccaugacaaguagguuuuagagcuaugcuguuug

SEQ ID NO: 186 - DNA, 40 bp, Artificial Sequence
  Chemically Synthesized
  modified_base (1)..(2): phosphorotioate nucleotide
  modified_base (38)..(39): phosphorotioate nucleotide
cggtgtgagagcctcgaaagaggctctcacatcgctactt

SEQ ID NO: 187 - DNA, 28 bp, Artificial Sequence
  Chemically Synthesized
  modified_base (1)..(2): phosphorotioate nucleotide
  modified_base (26)..(27): phosphorotioate nucleotide
gaggctctcacagaaatgtgagagcctc

SEQ ID NO: 188 - DNA, 40 bp, Artificial Sequence; identical to SEQ ID NO: 186
  Chemically Synthesized
  modified_base (1)..(2): phosphorotioate nucleotide
  modified_base (35)..(40): locked nucleic acid
  modified_base (35)..(40): locked nucleic acid
  modified_base (35)..(35): m5c
  modified_base (38)..(38): m5c
  modified_base (38)..(39): phosphorotioate nucleotide

SEQ ID NO: 189 - RNA, 41 nt, Artificial Sequence
  Chemically Synthesized
gcauuucucaguccuaaacaguuuuagagcuaugcuguuug

SEQ ID NO: 190 - RNA, 41 nt, Artificial Sequence
  Chemically Synthesized
gaguccgagcagaagaagaaguuuuagagcuaugcuguuug

SEQ ID NO: 191 - DNA, 40 bp, Artificial Sequence
  Chemically Synthesized
  modified_base (1)..(2): phosphorotioate nucleotide
  modified_base (35)..(40): locked nucleic acid
  modified_base (38)..(39): phosphorotioate nucleotide
cggtgtgagagcctcgaaagaggctctcacatcgtgttta

SEQ ID NO: 192 - DNA, 40 bp, Artificial Sequence
  Chemically Synthesized
  modified_base (1)..(2): phosphorotioate nucleotide
  modified_base (35)..(40): locked nucleic acid
  modified_base (35)..(40): locked nucleic acid
  modified_base (37)..(37): m5c
  modified_base (38)..(39): phosphorotioate nucleotide
  modified_base (40)..(40): m5c
cggtgtgagagcctcgaaagaggctctcacatcgttcttc

SEQ ID NO: 193 - DNA, 18 bp, Artificial Sequence
  Chemically Synthesized
acatcagcagctgttctg

SEQ ID NO: 194 - DNA, 18 bp, Artificial Sequence
  Chemically Synthesized
ggctgaaaggagagaact

SEQ ID NO: 195 - DNA, 26 bp, Artificial Sequence
  Chemically Synthesized
gccatccccttctgtgaatgttagac

SEQ ID NO: 196 - DNA, 23 bp, Artificial Sequence
  Chemically Synthesized
cggaatctaccaccccaggctct

(19) United States
(12) Patent Application Publication
(10) Pub. No.: US 2020/0255836 A1
(43) Pub. Date: Aug. 13, 2020

(54) NUCLEIC ACID APTAMER FOR INHIBITING ACTIVITY OF GENOME-EDITING ENZYME

(71) Applicant: NATIONAL INSTITUTE OF ADVANCED INDUSTRIAL SCIENCE AND TECHNOLOGY, Tokyo (JP)

(72) Inventors: Makoto Miyagishi, Tsukuba-shl, Ibaraki (JP); Yoshio KATO, Tsukuba-shi, Ibaraki (JP); Jing ZHAO, Tsukuba-shi, Ibaraki (JP)

(21) Appl. No.: 16/639,389

(22) PCT Filed: Aug. 17, 2018
(86) PCT No.: PCT/JP2018/030530

§ 371 Date: Feb. 14, 2020

(30) Foreign Application Priority Data
Aug. 18, 2017 (JP) 2017-157970

(51) Int. Cl. C12N 15/115; C12N 9/22

(52) U.S. Cl. CPC C12N 15/115; C12N 9/22; C12N 2310/315; C12N 2310/16; C12N 2310/20

(57) ABSTRACT

A nucleic acid aptamer inhibits the binding activity or enzymatic activity of a complex comprising guide RNA and nuclease against a target nucleic acid, the nucleic acid aptamer comprising the following regions (1) to (3): (1) a single-stranded guide RNA recognition region comprising a guide RNA-recognizing oligonucleotide including a sequence recognizing the guide RNA; (2) a neck region comprising a first neck oligonucleotide including the PAM sequence, and a second neck oligonucleotide including a sequence having complementarity to the PAM sequence; and (3) a double-stranded structure stabilization region comprising a first structure-stabilizing oligonucleotide and a second structure-stabilizing oligonucleotide, in which the region (1) is linked to the second neck oligonucleotide to form a loop structure or a flap structure, and the regions (2) and (3) are linked to each other to together form a stem structure.

### TECHNICAL FIELD

The present invention relates to a nucleic acid aptamer that inhibits activity of a genome-editing enzyme.

### BACKGROUND ART

Methods using Zinc finger, TALEN, or CRISPR-Cas9 are known as genome editing methods. Among them, a genome editing method using CRISPR-Cas9 is often adopted to research targeting a wide range of cells or species of organisms because of its high efficiency and convenience. CRISPR-Cas9 forms a complex with guide RNA, and recognizes and cleaves DNA having a sequence complementary to a part of the guide RNA and a PAM sequence adjacent thereto. The gene can be engineered (e.g., knocked out) by, e.g., base insertion or deletion that occurs during the process of repairing the cleavage site of the DNA.

Methods for introducing a foreign gene through homologous recombination or non-homologous end joining (NHEJ) exploiting the fact that the cleavage of DNA by CRISPR-Cas9 promotes a DNA repair mechanism have also been developed. Moreover, a single-nucleotide editing technique using dCas9 (Cas9 mutant lacking cleaving activity) and deaminase, and an epigenome editing technique using dCas9 and DNA (de)methylase have also been reported. For example, Patent Document 1 discloses a fusion protein of dCas9 and a heterologous functional domain (e.g., a transcriptional activation domain).

If a method for specifically inhibiting CRISPR-Cas9 can be established, genome editing would be controllable temporally and/or spatially, and various approaches as described above are applicable to various purposes. Cas9 is known to have an off-target effect of cleaving a nontarget site. If an enzymatic reaction time can be limited using an inhibitor of CRISPR-Cas9, it is expected that the off-target effect in genome editing can be reduced.

### CITATION LIST

### Patent Document

[Patent Document 1] JP 2016-512264 A

### SUMMARY OF INVENTION

### Technical Problem

The present invention has been made with an object to provide a nucleic acid aptamer that can inhibit the effect of a genome editing enzyme on a target sequence by inhibiting the binding of the genome editing enzyme to the target sequence.

### Solution to Problem

The present inventors have earnestly researched and, as a result, successfully obtained a nucleic acid aptamer that can inhibit activities of a genome editing enzyme by specifically binding thereto.

Specifically, according to one embodiment, the present invention provides a nucleic acid aptamer that inhibits binding activity or enzymatic activity of a complex comprising guide RNA and an nuclease against a target nucleic acid serving as a substrate of the complex, the nucleic acid aptamer comprising the following regions (1) to (3): (1) a single-stranded guide RNA recognition region comprising a guide RNA-recognizing oligonucleotide including a sequence recognizing the guide RNA; (2) a double-stranded neck region comprising a PAM sequence corresponding to the nuclease in one strand, the neck region comprising a first neck oligonucleotide including the PAM sequence, and a second neck oligonucleotide including a sequence having complementarity to the PAM sequence; and (3) a double-stranded structure stabilization region comprising a first structure-stabilizing oligonucleotide and a second structure-stabilizing oligonucleotide, wherein the region (1) is linked to the second neck oligonucleotide to form a flap structure, or the region (1) is linked to the first neck oligonucleotide and the second neck oligonucleotide to form a loop structure where the guide RNA-recognizing oligonucleotide in the region (1) is adjacent to the second neck oligonucleotide, and the region (2) and the region (3) are linked to each other to together form a stem structure.

The nuclease is preferably a nuclease of the CRISPR-Cas family.

The guide RNA-recognizing oligonucleotide in the region (1) preferably includes a 2-base to 30-base long sequence adjacent to the PAM sequence in the target nucleic acid.

The guide RNA-recognizing oligonucleotide in the region (1) more preferably includes a 3-base to 22-base long sequence adjacent to the PAM sequence in the target nucleic acid.

The region (1) is preferably 6 to 50 bases long.

The region (1) preferably comprises a bridged nucleic acid.

The region (2) may comprise a mismatch or a bulge.

Preferably, the first neck oligonucleotide in the region (2) is 5′-NGG-3′, and the complex is CRISPR-Cas9.

Preferably, the first neck oligonucleotide in the region (2) is 5′-TTTN-3′, and the complex is CRISPR-Cpf1.

The region (3) is preferably 4 base pairs long or longer.

The nucleic acid aptamer may comprise a phosphorothioate modification.

According to one embodiment, the present invention also provides a method for producing a nucleic acid aptamer inhibiting binding activity or enzymatic activity of a complex comprising guide RNA and nuclease against a target nucleic acid served as a substrate for the complex, the method comprising the steps of:
(1) determining a guide RNA-recognizing oligonucleotide including a sequence recognizing the guide RNA;
(2) determining a first neck oligonucleotide including a PAM sequence compatible with the nuclease, and a second neck oligonucleotide including a sequence having complementarity to the first neck oligonucleotide;
(3) determining a first structure-stabilizing oligonucleotide and a second structure-stabilizing oligonucleotide;
(4) adding the first structure-stabilizing oligonucleotide to the first neck oligonucleotide;
(5) adding the second structure-stabilizing oligonucleotide to the second neck oligonucleotide;
(6) linking the guide RNA-recognizing oligonucleotide to the second neck oligonucleotide; and
(7) synthesizing a nucleic acid comprising the sequences designed by the steps (1) to (6).

The method preferably further comprises the step of:
(5′) linking the first structure-stabilizing oligonucleotide to the second structure-stabilizing oligonucleotide.

Alternatively, the method preferably further comprises the step of:
(6′) linking the guide RNA-recognizing oligonucleotide to the first neck oligonucleotide either directly or via a linker oligonucleotide.

According to one embodiment, the present invention also provides a method for inhibiting binding activity or enzymatic activity of a complex comprising guide RNA and nuclease against a target nucleic acid served as a substrate of the complex, the method comprising the steps of:
(1) preparing a reaction solution containing the complex and the target nucleic acid; and
(2) adding the nucleic acid aptamer to the reaction solution.

According to one embodiment, the present invention also provides a method for intracellularly inhibiting binding activity or enzymatic activity of a complex comprising guide RNA and nuclease against a target nucleic acid served as a substrate of the complex, the method comprising the steps of:
(1) introducing the complex to a cell containing the target nucleic acid; and
(2) introducing the nucleic acid aptamer to the cell.

According to one embodiment, the present invention provides a genome editing method comprising the steps of:
(1) introducing a complex comprising guide RNA and nuclease of the CRISPR-Cas family to a cell; and
(2) introducing the nucleic acid aptamer to the cell.

### Advantageous Effects of Invention

The nucleic acid aptamer of the present invention can temporally and/or spatially control the effect of a genome editing enzyme on a target sequence by inhibiting the binding of the genome editing enzyme to the target sequence based on the CRISPR-Cas system. Furthermore, the aptamer is synthesized from nucleic acids and is therefore easily introduced into cells as compared to an antibody. Hence, the nucleic acid aptamer of the present invention exerts the following excellent effects and is useful.

(1) Use of the nucleic acid aptamer of the present invention can control the timing or accuracy of genome editing in vitro or in vivo (in cells or in live animals), and can reduce or circumvent unnecessary and unwanted effects of a genome editing enzyme complex on a target sequence.

(2) When cleaving a nucleic acid using a genome editing enzyme complex in vitro or in vivo (in cells or in live animal), use of the nucleic acid aptamer of the present invention at a low concentration, or use of a nucleic acid aptamer with attenuated binding specificity, can partially suppress the activity of the genome editing enzyme complex. As a result, the genome editing enzyme complex can act precisely only on the target sequence, and off-target effects can be minimized.

(3) When a plurality of Cas9/sgRNA complexes targeting different sequences are introduced at the same time into cells, only the activity of a particular Cas9/sgRNA complex can be inhibited.

(4) Linking of a functional nucleic acid recognizing an additional substance to the aptamer of the present invention allows its inhibitory activity on a genome editing enzyme complex to be freely controlled using the additional substance.

(5) If a target pathogen in phage therapy has the CRISPR-Cas system, the pathogen acquires immunity against phages, and the phages can therefore no longer infect the pathogen. However, use of the nucleic acid aptamer of the present invention can circumvent this problem. Use of the nucleic acid aptamer of the present invention to suppress the CRISPR-Cas system of the pathogen can sustain the ability of the phages to infect the pathogen, and can enhance the effect of the phage therapy.

(6) Combination use of an RNA-targeting CRISPR-Cas system-based diagnostic kit for viral infection and the nucleic acid aptamer of the present invention can reduce false-positive detection, and can enhance the accuracy of diagnosis.

### BRIEF DESCRIPTION OF DRAWINGS

FIG. 1 is a view showing results of evaluating candidate aptamers against Cas9, obtained by SELEX for their inhibitory effect on the cleavage at the target site by Cas9/sgRNA(GFPg1), in in vitro assay. The s21, s36 and s40 were found to have high inhibitory activity.

FIG. 2 is a view showing results of confirming the concentration dependence of the inhibitory activity of the candidate aptamers s21, s36 and s40 against the cleavage at the target site by Cas9/sgRNA(GFPg1), in in vitro assay.

FIG. 3 is a schematic diagram showing the secondary structure of s21.

FIG. 4 is a view showing results of evaluating s21 mutants with a truncated guide RNA recognition region (truncation mutants) for their inhibitory effect on the cleavage at the target site by Cas9/sgRNA(GFPg1), in in vitro assay.

FIG. 5 is a view showing results of evaluating s21 and s36 aptamer mutants having a mutation introduced in the guide RNA recognition region for their inhibitory effect on the cleavage at the target site by Cas9/sgRNA(GFPg1), in in vitro assay.

FIG. 6 is a view showing results of evaluating s21-2 aptamer mutants having a point mutation introduced in the guide RNA recognition region for their inhibitory effects on the cleavage at the target site by Cas9/sgRNA(GFPg1), in in vitro assay.

FIG. 7 is a view showing results of evaluating aptamers having varied lengths of the structure stabilization region of s21-2 for their inhibitory effect on the cleavage at the target site by Cas9/sgRNA(GFPg1), in in vitro assay.

FIG. 8 is a view showing results of evaluating aptamers having varied sequences of the structure stabilization region of s21-2 for their inhibitory effect on the cleavage at the target site by Cas9/sgRNA(GFPg1), in in vitro assay.

FIG. 9 is a view showing results of evaluating s21-2 aptamer mutants having a mutation introduced in the neck region for their inhibitory effect on the cleavage at the target site by Cas9/sgRNA(GFPg1), in to in vitro assay.

FIG. 10 is a view showing results of evaluating phosphorothioate-modified s21-2 aptamers for their inhibitory effect on the cleavage at the target site by Cas9/sgRNA(GFPg1), in in vitro assay.

FIG. 11 is a view showing results of evaluating the interaction of s21-based aptamers with Cas9, in gel shift assay.

FIG. 12 is a view showing results of evaluating the interaction of s21-based aptamers with Cas9, in surface plasmon resonance assay.

FIG. 13 is a schematic diagram showing the secondary structures of s21-2 (stem-loop-type aptamer) and s21-sf (stem-flap-type aptamer).

FIG. 14 is a view showing results of evaluating an s21-based stem-flap-type aptamer for its inhibitory effect on the cleavage at the target site by Cas9/sgRNA(GFPg1), in in vitro assay.

FIG. 15 is a view showing results of evaluating s21-2 for its inhibitory effects on Cas9/crRNA-targeting distinctive sequences, in in vitro assay.

FIG. 16 is a view showing results of evaluating antisense DNA and antisense RNA against a guide sequence in sgRNA for their inhibitory effect on the cleavage at the target site by Cas9/sgRNA(GFPg1), in in vitro assay.

FIG. 17 is a schematic diagram of a mechanism underlying the inhibition of Cas9/sgRNA by the aptamer.

FIG. 18 is a view showing results of evaluating stem-loop-type aptamers and stem-flap-type aptamers for their inhibitory effects on Cas9/crRNA(GFP332) or Cas9/crRNA(GFP373), in in vitro assay.

FIG. 19 is a view showing results of evaluating stem-loop-type aptamers for the influence of the length of the loop structure on their inhibitory effects in in vitro assay.

FIG. 20 is a view showing results of evaluating stem-flap-type aptamers for the crRNA sequence specificity of their inhibitory effect on Cas9/crRNA, in in vitro assay.

FIG. 21 is a view showing results of evaluating stem-flap-type aptamers for the influence of the length of the flap structure on their inhibitory effects, in in vitro assay.

FIG. 22 is a view showing results of evaluating stem-flap-type aptamers for the influence of base addition to an end opposite to the flap structure on their inhibitory effects, in in vitro assay.

FIG. 23 is a view showing results of evaluating stem-flap-type aptamers for their inhibitory effects on Cas9/crRNA targeting EGFR gene, in in vitro assay.

FIG. 24 is a view showing results of evaluating stem-flap-type aptamers for the influence of the length of the flap structure on their inhibitory effects, in in vitro assay.

FIG. 25 is a view showing results of evaluating stem-flap-type aptamers for their inhibitory effects on Cas9/crRNA targeting EpCAM gene, in in vitro assay.

FIG. 26 is a schematic diagram of a mechanism underlying the inhibition of Cpf1/crRNA by the stem-flap-type aptamer.

FIG. 27 is a view showing results of evaluating stem-flap-type aptamers for their inhibitory effects on the cleavage at the target site by Cpf1/crRNA(GFPa), in in vitro assay.

FIG. 28 is a view showing results of evaluating stem-flap-type aptamers for their inhibitory effects on Cpf1/crRNA targeting EGFR gene, in in vitro assay.

FIG. 29 is a view showing fluorescence microscope images for confirming the inhibitory effect of stem-flap-type aptamers on the genome editing by a Cas9/crRNA:tracrRNA complex targeting a target sequence-mScarlet reporter cassette integrated into the genome in cells.

FIG. 30 is a view showing fluorescence microscope images for confirming the inhibitory effect of an aptamer with an LNA-modified flap structure moiety on the genome editing by a Cas9/crRNA:tracrRNA complex targeting a target sequence-mScarlet reporter cassette integrated into the genome in cells.

FIG. 31 is a graph showing results of quantifying the collected cells shown in FIG. 30 by FACS.

FIG. 32 is a view showing fluorescence microscope images for confirming the concentration dependence of the inhibitory effect of an aptamer with an LNA-modified flap structure moiety on the genome editing by a Cas9/crRNA:tracrRNA complex targeting a target sequence-mScarlet reporter cassette integrated into the genome in cells.

FIG. 33 is a graph showing results of quantifying the collected cells shown in FIG. 32 by FACS.

FIG. 34 is a view showing results of evaluating aptamers for their inhibitory effects on the genome editing by a Cas9/crRNA:tracrRNA complex targeting endogenous genes, on the basis of the detection of indels.

### DESCRIPTION OF EMBODIMENTS

Hereinafter, the present invention will be described in detail. However, the present invention is not limited by embodiments described in the present specification.

According to the first embodiment, the present invention provides a nucleic acid aptamer inhibiting the binding activity or enzymatic activity of a complex comprising guide RNA and nuclease against a target nucleic acid served as a substrate of the complex, the nucleic acid aptamer comprising the following regions (1) to (3):
(1) a single-stranded guide RNA recognition region comprising a guide RNA-recognizing oligonucleotide including a sequence recognizing the guide RNA;
(2) a double-stranded neck region comprising a PAM sequence compatible with the nuclease in one strand, the neck region comprising a first neck oligonucleotide including the PAM sequence, and a second neck oligonucleotide including a sequence having complementarity to the PAM sequence; and
(3) a double-stranded structure stabilization region comprising a first structure-stabilizing oligonucleotide and a second structure-stabilizing oligonucleotide, wherein the region (1) is linked to the second neck oligonucleotide to form a flap structure, or the region (1) is linked to the first neck oligonucleotide and the second neck oligonucleotide to form a loop structure where the guide RNA-recognizing oligonucleotide in the region (1) is adjacent to the second neck oligonucleotide, and the region (2) and the region (3) are linked to each other to together form a stem structure.

In the present invention, the “nucleic acid aptamer” means a nucleic acid molecule that can specifically bind to a target molecule (in the present invention, a complex comprising guide RNA and nuclease) with high affinity. The nucleic acid aptamer can have an inhibitory effect on the activity of a target molecule by specifically binding to the target molecule. The nucleic acid constituting the nucleic acid aptamer is not particularly limited and can be, for example, DNA, RNA, or a modified nucleic acid, only one or two or more in combination of which can constitute the nucleic acid aptamer. Accordingly, the nucleic acid aptamer according to the present invention can be a DNA aptamer, an RNA aptamer, a DNA/RNA chimeric nucleic acid aptamer, an aptamer comprising a modified nucleic acid in a portion of any of these aptamers, or the like. Preferably, the nucleic acid aptamer according to the present invention is a DNA aptamer.

In the present specification, the modified nucleic acid refers to a nucleic acid constituted by a non-natural nucleotide, or a non-natural nucleic acid. In this context, the “non-natural nucleotide” refers to a nucleotide containing a non-naturally occurring artificial chemical modification in a base or a sugar, and has properties and/or a structure similar to those of a natural nucleotide. Various non-natural nucleotides are known in the art. Examples thereof include non-natural nucleotides comprising abasic nucleoside, arabinonucleoside, 2′-deoxyuridine, α-deoxyribonucleoside, β-L-deoxyribonucleoside, or other nucleosides having a modified sugar (e.g., substituted pentose (2′-O-methylribose, 2′-deoxy-2′-fluororibose, 3′-O-methylribose, and 1′,2′-deoxyribose), arabinose, substituted arabinose sugar, substituted hexose, and α-anomeric sugar). In the present specification, the non-natural nucleotide may be a nucleotide comprising a base analog or a modified base. Examples of the base analog include a 2-oxo(1H)-pyridin-3-yl group, a 5-substituted 2-oxo(1H)-pyridin-3-yl group, a 2-amino-6-(2-thiazolyl)purin-9-yl group, a 2-amino-6-(2-thiazolyl)purin-9-yl group, and a 2-amino-6-(2-oxazolyl)purin-9-yl group. Examples of the modified base include modified pyrimidine (e.g., 5-hydroxycytosine, 5-fluorouracil, and 4-thiouracil), modified purine (e.g., 6-methyladenine and 6-thioguanosine), and other heterocyclic bases. In the present specification, the “non-natural nucleic acid” refers to a nucleic acid analog having a non-naturally occurring artificial chemical modification introduced in its structure, and has properties and/or a structure similar to those of a natural nucleic acid. Examples of the non-natural nucleic acid include peptide nucleic acid (PNA), peptide nucleic acid having a phosphate group (PHONA), bridged nucleic acid, morpholino nucleic acid, and triazole-linked nucleic acid. Further examples thereof include methyl phosphonate type DNA/RNA, phosphorothioate type DNA/RNA, phosphoramidate type DNA/RNA, and 2′-O-methyl type DNA/RNA.

The modified nucleic acid for use in the nucleic acid aptamer of the present invention is preferably a bridged nucleic acid and/or a phosphorothioate-modified nucleic acid. The modified nucleic acid may be contained in any region of the nucleic acid aptamer of the present invention. As mentioned below in detail, particularly preferably, a bridged nucleic acid is contained in the guide RNA recognition region of the nucleic acid aptamer.

In the present invention, the “complex comprising guide RNA and nuclease” means a complex that induces the nuclease specifically for a site to be recognized by the guide RNA, and can interact with a nucleic acid. Hereinafter, the complex comprising guide RNA and nuclease is referred to as a “gRNA/nuclease complex” in the present specification. The gRNA/nuclease complex according to the present invention is originally derived from the mechanism of acquired immunity called CRISPR-Cas (clustered regularly interspaced short palindromic repeats and CRISPR-associated proteins) in prokaryotes. At present, the CRISPR-Cas system is broadly classified into two classes on the basis of the type of Cas, and further classified into 6 types (types I to VI: types I, III and IV for class 1; and types II, V and VI for class 2). The gRNA/nuclease complex according to the present invention may be derived from any CRISPR-Cas system thereamong.

Thus, the “nuclease” according to the present invention may be arbitrary nuclease of the CRISPR-Cas family. Nonlimiting examples of the nuclease of the CRISPR-Cas family include Cas1, Cas1B, Cas2, Cas3, Cas4, Cas5, Cash, Cas7, Cas8, Cas9 (formerly called Csn1 or Csx12), Cas10, Cas12, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4, and their homologs.

The nuclease according to the present invention may be RNA-guided nuclease (RGN) other than Cas nuclease, and can be a freely chosen nuclease derived from an freely chosen species of organism, as long as the nuclease is induced by guide RNA and can interact with a target site in a nucleic acid. The nuclease according to the present invention may have an artificial modification or mutation, as long as the nuclease maintains its function of being induced by guide RNA and interacting with a target site in a nucleic acid.

The nuclease according to the present invention may be any DNA nuclease or RNA nuclease, and is particularly preferably a nuclease for use in genetic engineering in a genome editing technique. Specifically, the nuclease according to the present invention is preferably a nuclease of the CRISPR-Cas family, particularly preferably Cas9 or Cpf1 (also called Cas12a) (Takashi Yamano et al., “Crystal Structure of Cpf1 in Complex with Guide RNA and Target DNA”, Cell, doi:10.1016/j.cell.2016.04.003). Preferred examples of the RNA nuclease according to the present invention include Cas13a.

The nuclease according to the present invention may be an endonuclease, or may be a nickase. The nuclease according to the present invention may have lost its cleaving activity due to a mutation. Specifically, when Cas9 is taken as an example, the nuclease according to the present invention can include all types of endonuclease wild-type Cas9, nickase Cas9 (D10A), and dCas9 lacking cleaving activity. The nuclease according to the present invention can further include a fusion protein of such mutated nuclease and an additional functional domain (e.g., a transcriptional activation domain, a transcriptional suppression domain, and a cytidine deaminase).

In the present invention, the “guide RNA” means RNA that comprises a guide sequence complementary to a target nucleic acid, and has a function of guiding the gRNA/nuclease complex to the target nucleic acid and allowing the complex to specifically bind thereto. The structure of the guide RNA is not particularly limited as long as the guide RNA comprises a guide sequence and has the function. When the gRNA/nuclease complex according to the present invention is, for example, the type I CRISPR-Cas system, the guide RNA can be CRISPR RNA (crRNA). When the gRNA/nuclease complex according to the present invention is the type II CRISPR-Cas system, the guide RNA can be dual RNA of crRNA and trans-activating crRNA (tracrRNA). The guide RNA according to the present invention may be single-stranded guide RNA (sgRNA) comprising crRNA and tracrRNA linked through a linker. For example, in the CRISPR-Cas system, the crRNA has a guide sequence approximately 16 bases to 24 bases long that is complementary to a target nucleic acid, and a repeat region, and the tracrRNA has an anti-repeat region complementary to the repeat region. The crRNA and the tracrRNA form a double strand.

The nucleic acid aptamer of the present invention comprises, as a first region, a single-stranded guide RNA recognition region comprising a guide RNA-recognizing oligonucleotide including a sequence recognizing the guide RNA contained in the gRNA/nuclease complex. The “guide RNA recognition region” in the nucleic acid aptamer of the present invention means a functional structural unit for the interaction of the nucleic acid aptamer with the guide RNA contained in the gRNA/nuclease complex of interest.

The guide RNA recognition region in the nucleic acid aptamer of the present invention comprises a guide RNA-recognizing oligonucleotide including a sequence recognizing the guide RNA contained in the gRNA/nuclease complex. In this context, the “sequence recognizing the guide RNA” means that the sequence forms base pairs with at least a portion of the guide RNA. The base pairs can include not only G:C and A:T but wobble base pairs such as G:T or G:U. In this context, the phrase “at least a portion” of the guide RNA means a length of 2 bases to the full length of the guide RNA. Thus, the “sequence recognizing the guide RNA” in the nucleic acid aptamer of the present invention has complementarity to a length of 2 bases to the full length of the guide RNA. In this respect, the guide RNA-recognizing oligonucleotide need not have perfect or complete (i.e., 100%) complementarity to a length of 2 bases to the full length of the guide RNA, and can have complementarity sufficient for the interaction of the guide RNA-recognizing oligonucleotide with at least a portion of the guide RNA.

Thus, the “guide RNA-recognizing oligonucleotide” in the nucleic acid aptamer of the present invention can have at least 80%, 90%, 95%, 99%, or 100% sequence complementarity to a length of 2 bases to the full length of the guide RNA. In other words, the “guide RNA-recognizing oligonucleotide” in the nucleic acid aptamer of the present invention can be a sequence derived from a sequence completely complementary to a length of 2 bases to the full length of the guide RNA by the insertion, deletion or substitution of several (e.g., 1, 2, 3, 4 and 5) bases. The sequence complementarity can be calculated using a calculation algorithm (NCBI BLAST, etc.) commonly used in the art.

When the guide RNA-recognizing oligonucleotide in the nucleic acid aptamer of the present invention includes a sequence having complementarity to a partial sequence of the guide RNA, the partial sequence can be selected from any portion of the guide RNA, and is preferably selected from the guide sequence in the guide RNA. In this context, the partial sequence to be selected from the guide sequence is preferably complementary to a portion adjacent to a PAM sequence in a target nucleic acid of the gRNA/nuclease complex. Specifically, the guide RNA-recognizing oligonucleotide in the nucleic acid aptamer of the present invention can include, for example, a sequence of 2 bases or more, 3 bases or more, or 4 bases or more adjacent to a PAM sequence of a target nucleic acid, and preferably includes a 2-base to 30-base long sequence adjacent to a PAM sequence of a target nucleic acid, particularly preferably a 3-base to 22-base long sequence adjacent to a PAM sequence of a target nucleic acid, most preferably a 4-base to 15-base long sequence adjacent to a PAM sequence of a target nucleic acid.

The guide RNA recognition region in the nucleic acid aptamer of the present invention may be constituted only by the guide RNA-recognizing oligonucleotide, or may comprise a linker oligonucleotide. The linker oligonucleotide can have a freely chosen sequence, and preferably has a sequence that forms no base pair with the guide RNA-recognizing oligonucleotide and also preferably has an AT-rich sequence. The linker oligonucleotide can be linked to either one end or both ends of the guide RNA-recognizing oligonucleotide, and is preferably linked directly to the second neck oligonucleotide mentioned below without the mediation of a linker sequence. The linker oligonucleotide can have a freely chosen length as long as the function of the guide RNA recognition region is maintained. Thus, the guide RNA recognition region can be, for example, 6 to 50 bases long, preferably 7 to 25 bases long, particularly preferably 7 to 15 bases long, in total.

The nucleic acid aptamer of the present invention may be constituted by a freely chosen nucleic acid such as DNA, RNA, or modified nucleic acid, as mentioned above, and particularly preferably comprises a bridged nucleic acid in the guide RNA recognition region. The guide RNA recognition region comprising a bridged nucleic acid can further improve the inhibitory activity of the nucleic acid aptamer against the complex. BNA (bridged nucleic acid) and 2′,4′-BNA (also called LNA (locked nucleic acid)) as well as their analogs (e.g., amino-LNA, thio-LNA, α-L-oxy-LNA, ENA (2′-0,4′-C-ethylene-bridged nucleic acid), AmNA (amido-bridged nucleic acid), GuNA (guanidine-bridged nucleic acid), scpBNA (2′-0,4′-C-spirocyclopropylene-bridged nucleic acid), cEt-BNA (constrained ethyl-bridged Nucleic Acid), 3′-amino-2′,4′-BNA, 5′-amino-2′,4′-BNA, PrNA (2′-0,4′-C-propylene-bridged nucleic acid), 2′,4′-BNA^{NC} (2′-0,4′-C-aminomethylene-bridged nucleic acid), and 2′,4′-BNA^{COC} (2′-0,4′-C-methyleneoxymethylene-bridged nucleic acid)) can be used as the bridged nucleic acid. The percentage of the bridged nucleic acid contained in the guide RNA recognition region is not particularly limited, and can be, for example, 50% or more, 70% or more, 80% or more, 90% or more, or 100%.

The nucleic acid aptamer of the present invention comprises, as a second region, a double-stranded neck region comprising a PAM sequence compatible with the nuclease in one strand, the neck region comprising a first neck oligonucleotide including the PAM sequence, and a second neck oligonucleotide including a sequence having complementarity to the PAM sequence.

The first neck oligonucleotide in the nucleic acid aptamer of the present invention includes a PAM sequence that is recognized by the nuclease in the gRNA/nuclease complex. The “PAM (protospacer adjacent motif)” is a 2-base to 8-base long sequence which is necessary for the interaction of the nuclease with a target site in a target nucleic acid, and is located adjacent to a sequence targeted by the guide RNA, in the target nucleic acid (the PAM is absent in the targeting guide RNA). The PAM sequence differs depending on the bacterial species from which the nuclease is derived, or the type and/or subtype of the nuclease. Table 1 given below shows exemplary PAM sequences that are recognized by Cas9. For example, the PAM sequence that is recognized by Cas9 derived from S. pyogenes is known to be 5′-NGG.

TABLE 1

| Bacterial species | Subtype | PAM sequence |
|---|---|---|
| S. pyogenes | II | 5′-NGG |
| S. solfataricus | I-A1 | 5′-CCN |
| S. solfataricus | I-A2 | 5′-TCN |
| H. walsbyi | I-B | 5′-TTC |
| E. coli | I-E | 5′-AWG |
| E. coli | I-F | 5′-CC |
| P. aeruginosa | I-F | 5′-CC |
| S. thermophilus | II-A | 5′-NNAGAA |
| S. agelactiae | II-A | 5′-NGG |

Nucleases mutated so as to recognize various different PAM sequences have also been prepared. There are many reports on such nucleases and PAM sequences (see e.g., Cebrian-Serrano, A. & Davies, B., “CRISPR-Cas orthologues and variants: optimizing the repertoire, specificity and delivery of genome engineering tools”, Mamm. Genome (2017). doi:10.1007/s00335-017-9697-4; and Murovec J, Pirc Z, Yang B., “New variants of CRISPR RNA-guided genome editing enzymes.”, Plant Biotechnol. J. (2017) April 1. doi:10.1111/pbi.12736). The nucleic acid aptamer of the present invention may be directed to any of the nucleases and the PAM sequences described above. A PAM sequence suitable for the targeted gRNA/nuclease complex can be selected and used as the first neck oligonucleotide.

When the targeted gRNA/nuclease complex comprises an RNA nuclease, such as Cas13a, the sequence of a protospacer flanking site (PFS) can be used as the first neck oligonucleotide. The PFS sequence is a sequence functionally similar to the PAM sequence, and is located adjacent to a sequence targeted by the guide RNA, in target RNA. Thus, in the present invention, the PFS sequence may be included in the PAM sequence that can be used as the first neck oligonucleotide.

As already mentioned above, the nuclease contained in the gRNA/nuclease complex according to the present invention is preferably Cas9 or Cpf1. Thus, in the nucleic acid aptamer of the present invention, a PAM sequence compatible therewith (5′-NGG and 5′-TTTN, respectively) is preferably used as the first neck oligonucleotide.

The second neck oligonucleotide in the nucleic acid aptamer of the present invention includes a sequence having complementarity to the PAM sequence used as the first neck oligonucleotide. In this respect, the second neck oligonucleotide preferably has 100% complementarity to the PAM sequence used as the first neck oligonucleotide, and can comprise one or two mismatches or bulges as long as the first neck oligonucleotide and the second neck oligonucleotide can form a double strand.

In the nucleic acid aptamer of the present invention, the single-stranded guide RNA recognition region is linked to at least the second neck oligonucleotide. When the single-stranded guide RNA recognition region is linked only to the second neck oligonucleotide, the single-stranded guide RNA recognition region forms a flap structure. When the single-stranded guide RNA recognition region is linked to both the first neck oligonucleotide and the second neck oligonucleotide, the single-stranded guide RNA recognition region forms a loop structure. In any of the cases, the single-stranded guide RNA recognition region is linked such that the guide RNA-recognizing oligonucleotide contained therein is adjacent to the second neck oligonucleotide.

The nucleic acid aptamer of the present invention comprises, as a third region, a double-stranded structure stabilization region comprising a first structure-stabilizing oligonucleotide and a second structure-stabilizing oligonucleotide. The first structure-stabilizing oligonucleotide can include a freely chosen nucleic acid sequence, and the second structure-stabilizing oligonucleotide can have a freely chosen nucleic acid sequence as long as the sequence can form a double-strand with the first structure-stabilizing oligonucleotide. Thus, the first structure-stabilizing oligonucleotide and the second structure-stabilizing oligonucleotide can have at least 70% or more, 80% or more, 90% or more, 95% or more, or 100% complementarity. The double-stranded structure stabilization region may comprise one or more (e.g., 1, 2, 3, 4, 5 or more) mismatches or bulges.

The first structure-stabilizing oligonucleotide is added to the first neck oligonucleotide, and the second structure-stabilizing oligonucleotide is added to the second neck oligonucleotide. These oligonucleotides are hybridized with each other to form a double-stranded structure stabilization region, thereby stabilizing the entire structure of the nucleic acid aptamer. Thus, the double-stranded structure stabilization region can have a freely chosen length as long as the nucleic acid aptamer of the present invention maintains the structures and functions of the neck region and the guide RNA recognition region. The double-stranded structure stabilization region can be, for example, 4 base pairs long or longer, 6 base pairs long or longer, or 8 base pairs long or longer, and is preferably 6 to 20 base pairs long.

The first structure-stabilizing oligonucleotide and the second structure-stabilizing oligonucleotide may have the same length, and may have different lengths. Thus, the free end (i.e., the end opposite to the end linked with the neck region) of the structure stabilization region may be a blunt end, or may be a sticky end (5′ protruding or 3′ protruding end). Alternatively, the bases at the free end of the structure stabilization region may be mutually linked.

The nucleic acid aptamer of the present invention can be produced by combining the single-stranded guide RNA recognition region, the double-stranded neck region, and the double-stranded structure stabilization region designed according to the description above.

Specifically, according to the second embodiment, the present invention provides a method for producing a nucleic acid aptamer inhibiting the binding activity or enzymatic activity of a complex comprising guide RNA and nuclease against a target nucleic acid serving as a substrate of the complex, the method comprising the steps of:
(1) determining a guide RNA-recognizing oligonucleotide including a sequence recognizing the guide RNA;
(2) determining a first neck oligonucleotide including a PAM sequence compatible with the nuclease, and a second neck oligonucleotide including a sequence having complementarity to the first neck oligonucleotide;
(3) determining a first structure-stabilizing oligonucleotide and a second structure-stabilizing oligonucleotide;
(4) adding the first structure-stabilizing oligonucleotide to the first neck oligonucleotide;
(5) adding the second structure-stabilizing oligonucleotide to the second neck oligonucleotide;
(6) linking the guide RNA-recognizing oligonucleotide to the second neck oligonucleotide; and
(7) synthesizing a nucleic acid comprising the sequences designed by the steps (1) to (6).

In this context, in the CRISPR-Cas system, the arrangement (positional relationship) of the PAM sequence in a target gene and a sequence targeted by the guide RNA (hereinafter, referred to as a “guide RNA-targeted sequence”) differs depending on the type of Cas. For example, Cas9 recognizes a PAM sequence downstream (i.e., adjacent to the 3′ end of the guide RNA-targeted sequence) of the guide RNA-targeted sequence, and Cpf1 recognizes a PAM sequence upstream (i.e., adjacent to the 5′ end of the guide RNA-targeted sequence) of the guide RNA-targeted sequence. Hence, the order in which the guide RNA-recognizing oligonucleotide and the first and/or second neck oligonucleotide in the nucleic acid aptamer of the present invention are arranged in the 5′→3′ direction is appropriately changeable by the type of the nuclease contained in the gRNA/nuclease complex to which the nucleic acid aptamer is directed.

Thus, for example, in the case of producing a nucleic acid aptamer in which the single-stranded guide RNA recognition region is linked to both the first and second neck oligonucleotides (i.e., the single-stranded guide RNA recognition region forms a loop structure),
(i) a nucleic acid aptamer directed to a complex comprising nuclease, such as Cas9, which recognizes a PAM sequence downstream of the guide RNA-targeted sequence comprises:
second structure-stabilizing oligonucleotide-second neck oligonucleotide-guide RNA-recognizing oligonucleotide-(linker oligonucleotide—)first neck oligonucleotide-first structure-stabilizing oligonucleotide
   and linked in this order in the 5′→3′ direction; and
(ii) a nucleic acid aptamer directed to a complex comprising nuclease, such as Cpf1, which recognizes a PAM sequence upstream of the guide RNA-targeted sequence comprises:
first structure-stabilizing oligonucleotide-first neck oligonucleotide-(linker oligonucleotide—)guide RNA-recognizing oligonucleotide-second neck oligonucleotide-second structure-stabilizing oligonucleotide
   and linked in this order in the 5′→3′ direction.
   In the description above, the term “(linker oligonucleotide—)” means a freely chosen linker oligonucleotide. Thus, the method of the present embodiment may further comprise the step of (6′) linking the guide RNA-recognizing oligonucleotide to the first neck oligonucleotide either directly or via a linker oligonucleotide.

On the other hand, for example, in the case of producing a nucleic acid aptamer in which the single-stranded guide RNA recognition region is linked only to the second neck oligonucleotide (i.e., the single-stranded guide RNA recognition region forms a flap structure),
(i) a nucleic acid aptamer directed to a complex comprising nuclease, such as Cas9, which recognizes a PAM sequence downstream of the guide RNA-targeted sequence comprises:
first neck oligonucleotide-first structure-stabilizing oligonucleotide(−)second structure-stabilizing oligonucleotide-second neck oligonucleotide-guide RNA-recognizing oligonucleotide
   and linked in this order in the 5′→3′ direction; and
(ii) a nucleic acid aptamer directed to a complex comprising nuclease, such as Cpf1, which recognizes a PAM sequence upstream of the guide RNA-targeted sequence comprises:
guide RNA-recognizing oligonucleotide-second neck oligonucleotide-second structure-stabilizing oligonucleotide(−)first structure-stabilizing oligonucleotide-first neck oligonucleotide
   and linked in this order in the 5′→3′ direction.
   In the description above, the symbol “(−)” means a freely chosen linking to the free end of the structure stabilization region. Thus, the method of the present embodiment may further comprise the step of (5′) linking the first structure-stabilizing oligonucleotide to the second structure-stabilizing oligonucleotide.

The nucleic acid aptamer designed as described above can be produced by a method known in the art. Specifically, the nucleic acid aptamer can be chemically synthesized by, for example, an amidite method or a phosphoramidite method (see e.g., Nucleic Acid (Vol. 2) [1] Synthesis and Analysis of Nucleic Acid (Editor: Yukio Sugiura, Hirokawa Publishing Company)). Alternatively, the nucleic acid aptamer may be biosynthesized by a method using RNA polymerase or a gene engineering approach using DNA polymerase. For example, when the nucleic acid aptamer of the present invention is an RNA aptamer, the RNA aptamer can be prepared by chemically synthesizing template DNA comprising a promoter sequence (e.g., T7 promoter) of RNA polymerase, and transcribing the template DNA with the RNA polymerase. For example, when the nucleic acid aptamer of the present invention is DNA aptamer, the DNA aptamer can be prepared by chemically synthesizing a DNA template, and amplifying the template DNA by PCR.

In the case of producing a nucleic acid aptamer in which the single-stranded guide RNA recognition region is linked only to the second neck oligonucleotide (i.e., the single-stranded guide RNA recognition region forms a flap structure), the nucleic acid aptamer of the present invention may be prepared by the synthesis and subsequent hybridization of two partial nucleic acids of the aptamer, or may be prepared by linking the first structure-stabilizing oligonucleotide and the second structure-stabilizing oligonucleotide at the free end (i.e., an end opposite to the end linked with the neck region) of the structure stabilization region to synthesize one nucleic acid. The nucleic acid aptamer may be prepared, for example, by synthesizing a first partial nucleic acid of the aptamer including first neck oligonucleotide-first structure-stabilizing oligonucleotide and a second partial nucleic acid of the aptamer including second structure-stabilizing oligonucleotide-second neck oligonucleotide-guide RNA-recognizing oligonucleotide, and then hybridizing both, or may be preparing by synthesizing first neck oligonucleotide-first structure-stabilizing oligonucleotide-second structure-stabilizing oligonucleotide-second neck oligonucleotide-guide RNA-recognizing oligonucleotide, as one nucleic acid.

The nucleic acid aptamer of the present invention can inhibit the action of the gRNA/nuclease complex on a target sequence by specifically binding to the complex. Hence, the nucleic acid aptamer of the present invention can be used alone in combination with various existing methods based on the CRISPR-Cas system, including genome editing, thereby freely controlling the timing or accuracy thereof, and is therefore useful.

Specifically, according to the third embodiment, the present invention provides a method for inhibiting the binding activity or enzymatic activity of a complex comprising guide RNA and nuclease against a target nucleic acid served as a substrate of the complex, the method comprising the steps of: (1) preparing a reaction solution containing the complex and the target nucleic acid; and (2) adding the nucleic acid aptamer to the reaction solution.

In the method of the present embodiment, a reaction solution containing the gRNA/nuclease complex and the target nucleic acid of the complex is prepared. The composition of the reaction solution is not particularly limited as long as the reaction solution is suitable for the enzymatic activity of the nuclease. The composition of the reaction solution can be suitably determined in accordance with the composition of a reaction solution for use in an already established genome editing method. The reaction solution can be prepared, for example, by adding the gRNA/nuclease complex and the target nucleic acid of the complex to an aqueous solvent containing a buffer (e.g., 1 to 100 mM HEPES (pH 7.0 to pH 8.0) and 1 to 100 mM Tris (pH 7.0 to pH 8.0)) and/or a salt (e.g., 50 to 300 mM NaCl, 50 to 300 mM KCl, and 0 to 100 mM MgCl₂). The final concentration of the gRNA/nuclease complex in the reaction solution can be in the range of, for example, 10 to 300 nM. The final concentration of the target nucleic acid in the reaction solution can be in the range of, for example, 1 to 1000 nM. The reaction time between the gRNA/nuclease complex and the target nucleic acid of the complex can be appropriately determined according to the type of the nuclease used. The reaction can be performed, for example for 5 minutes to 24 hours.

Subsequently, the nucleic acid aptamer is added to the reaction solution. The amount of the nucleic acid aptamer added can be appropriately determined in the range of, for example, 0.1 to 1000 nM in terms of the final concentration. The inhibition reaction with the nucleic acid aptamer can be preferably performed for 5 minutes to 24 hours.

In the method of the present embodiment, the gRNA/nuclease complex and the nucleic acid aptamer can be added sequentially or simultaneously to the reaction solution. For example, after reaction between the gRNA/nuclease complex and the target nucleic acid, the nucleic acid aptamer can be added thereto. This can stop excessive enzymatic reaction of the complex. Alternatively, the nucleic acid aptamer may be added together with the preparation of the reaction solution containing the gRNA/nuclease complex and the target nucleic acid. This can control the degree or accuracy of the enzymatic reaction of the complex. Thus, all the gRNA/nuclease complex, the target nucleic acid of the complex, and the nucleic acid aptamer may be added at the same time to the aqueous solvent containing a buffer and/or a salt.

According to the fourth embodiment, the present invention provides a method for inhibiting the binding activity or enzymatic activity of a complex comprising guide RNA and nuclease against a target nucleic acid served as a substrate of the complex, the method comprising the steps of: (1) introducing the complex to a cell containing the target nucleic acid; and (2) introducing the nucleic acid aptamer to the cell.

In the method of the present embodiment, the gRNA/nuclease complex is introduced to a cell containing the target nucleic acid. The cell is not particularly limited as long as the cell contains the target nucleic acid of interest. Cells of any species of organism such as a prokaryote (e.g., E. coli), a fungus (e.g., an yeast), an insect, a plant, and an animal can be used. The cells in the method of the present embodiment are preferably cells derived from a plant or an animal, particularly preferably cells derived from a mammal such as a human. The type of the animal cells is not particularly limited, and cells isolated from a freely chosen tissue, fertilized eggs, cultured cells, or the like can be used. The target nucleic acid contained in the cells may be an endogenous nucleic acid such as genomic DNA or mitochondrial DNA, or may be an exogenous nucleic acid such as a plasmid vector.

The introduction of the gRNA/nuclease complex to the cells can be performed according to the protocol of an already established genome editing method. For example, gRNA and nuclease prepared in advance can be introduced into the cells by lipofection, microinjection, electroporation, or the like. Alternatively, an expression vector containing a nucleic acid encoding the gRNA and/or the nuclease may be introduced into the cells, and then, the cells can be cultured so that the gRNA/nuclease complex is expressed in the cells. A suitable viral vector or non-viral vector can be selected as the expression vector according to the type of the cells to which the gRNA/nuclease complex is introduced.

Subsequently, the nucleic acid aptamer is introduced to the cell. The introduction of the nucleic acid aptamer to the cells can be performed by a method known in the art according to the type of the cells, and can be performed by, for example, lipofection, microinjection, or electroporation. The cells after the introduction of the nucleic acid aptamer is preferably cultured for 1 to 7 days under suitable culture conditions according to the type thereof.

In the method of the present embodiment, the nucleic acid aptamer and the gRNA/nuclease complex can be introduced sequentially or at the same time into the cells. For example, the nucleic acid aptamer can be introduced into the cells 1 to 6 hours after the introduction of the gRNA/nuclease complex. This can suppress excessive enzymatic activity of the complex. Alternatively, the nucleic acid aptamer and the gRNA/nuclease complex may be introduced at the same time into the cells. This can control the degree or accuracy of the enzymatic reaction of the complex.

According to the fifth embodiment, the present invention provides a genome editing method comprising the steps of: (1) introducing a complex comprising guide RNA and nuclease of the CRISPR-Cas family to a cell; and (2) introducing the nucleic acid aptamer to the cell.

In the method of the present embodiment, the genome editing is performed using genomic DNA, as the target nucleic acid according to the fourth embodiment, and a gRNA/Cas nuclease complex, as the gRNA/nuclease complex according to the fourth embodiment. Thus, the cells that can be used in the method of the present embodiment is the same as that according to the fourth embodiment. The introduction of the gRNA/Cas nuclease complex and/or the nucleic acid aptamer into the cells can be performed by the same procedure as that in the method of the fourth embodiment.

The methods of the third to fifth embodiments are useful because the methods can freely control the timing, accuracy, degree, etc. of various existing methods based on the CRISPR-Cas system, including genome editing.

### EXAMPLES

Hereinafter, the present invention will be further described with reference to specific Examples. However, the present invention is not limited by the disclosure given below. The contents of documents cited herein are incorporated herein by reference.

### Example 1

### Acquirement of Nucleic Acid Aptamer Against Cas9

Nucleic acid aptamers against His-Cas9 protein prepared in an E. coli expression system were obtained as nucleic acid aptamers against Cas9 protein by SELEX. The details are as described below.

His-Cas9 (Streptococcus pyogenes Cas9) protein was bound to His-binding magnetic beads (Thermo Fisher Scientific Inc., 10103D), which were then washed with a binding buffer (10 mM Tris/HCl pH 7.4, 150 mM NaCl, 1 mM MgCl₂, and tRNA). Then, chemically synthesized nucleic acid libraries (N17, N19, N21, and N23) and the magnetic beads bound with the His-Cas9 protein were incubated at room temperature for 1 hour in a binding buffer. Then, the mixtures were washed with a binding buffer three times, followed by elution at 98° C. for 3 minutes to recover nucleic acids bound with the His-Cas9 protein. Five cycles of selection were performed. N-Rev and N-F were used as primers in an amplification step. The sequences amplified after the selection were analyzed with MiSeq from Illumina, Inc. Sequences of clusters having similar sequences were picked up from the obtained sequences of approximately 100,000 reads, and arbitrarily chosen candidate aptamer sequences were picked up from abundant sequences. The candidate aptamer sequences were chemically synthesized. In Examples given below, the candidate aptamers were examined for their inhibitory activities against Cas9. The sequences of the nucleic acid libraries, and the primer sequences are shown in Table 2. The picked up candidate sequences are shown in Tables 3A to 3C.

TABLE 2

| Library | Nucleotide sequence |
|---|---|
| N17 | GUGGAGAGGUTCTUACA NNNNNNNNNNNNNNNNN |
| | TGTGAGAGCCTCTCCGC |
| | (SEQ ID NO: 1) |
| N19 | GUGGAGAGGUTCTUACA NNNNNNNNNNNNNNNNNNN |
| | TGTGAGAGCCTCTCCGC |
| | (SEQ ID NO: 2) |
| N21 | GUGGAGAGGUTCTUACA NNNNNNNNNNNNNNNNNNNNN |
| | TGTGAGAGCCTCTCCGC |
| | (SEQ ID NO: 3) |
| N23 | GUGGAGAGGUTCTUACA |
| | NNNNNNNNNNNNNNNNNNNNNNN |
| | TGTGAGAGCCTCTCCGC |
| | (SEQ ID NO: 4) |
| Primer | Nucleotide sequence |
| N-Rev | CAAGCAGAAGACGGCATACGAGCTCTTCCGATCTGCGGAGAG |
| | GCTCTCACA |
| | (SEQ ID NO: 5) |
| N-F | AATGATACGGCGACCACCGAGATCTACACTCTTTCCCTACACG |
| | ACGCTCTTCCGATCTTCTACTGTGGAGAGGTTCTTACA |
| | (SEQ ID NO: 6) |

N represents G, A, T or C, and U represents deoxyuridine.

TABLE 3A

| Aptamer | Nucleotide sequence |
|---|---|
| s1 | GAGGCTCTCACATCGCCCTCCCTTGACGGTGTGAGAGCCTC |
| | (SEQ ID NO: 7) |
| s2 | GAGGCTCTCACATCACCCACCTTCAATGGTGTGAGAGCCTC |
| | (SEQ ID NO: 8) |
| s3 | GAGGCTCTCACACTTTGCCTTGCGGACCTTGTGAGAGCCTC |
| | (SEQ ID NO: 9) |
| s4 | GAGGCTCTCACATCATTAGGCGTAATTGGTGTGAGAGCCTC |
| | (SEQ ID NO: 10) |
| s5 | GAGGCTCTCACATCTATCGGCTTTACAGGTGTGAGAGCCTC |
| | (SEQ ID NO: 11) |
| s6 | GAGGCTCTCACATAAAAGGGGCAGGGTGGTGTGAGAGCCTC |
| | (SEQ ID NO: 12) |
| s7 | GAGGCTCTCACATTGGTCCCCTTTATCGGTGTGAGAGCCTC |
| | (SEQ ID NO: 13) |
| s8 | GAGGCTCTCACATTGGGGTGTACTTACGGTGTGAGAGCCTC |
| | (SEQ ID NO: 14) |
| s9 | GAGGCTCTCACATTAGGCGGCACCTCTAGTGTGAGAGCCTC |
| | (SEQ ID NO: 15) |
| s10 | GAGGCTCTCACATTGGGGTGTACTTACGGTGTGAGAGCCTC |
| | (SEQ ID NO: 16) |
| s11 | GAGGCTCTCACATTCACTATACCCTTGATTGTGAGAGCCTC |
| | (SEQ ID NO: 17) |
| s12 | GAGGCTCTCACATGTCCTAACCTCTCCGGTGTGAGAGCCTC |
| | (SEQ ID NO: 18) |
| s13 | GAGGCTCTCACAACTCAGCCCTCCCAGGGTGTGAGAGCCTC |
| | (SEQ ID NO: 19) |
| s14 | GAGGCTCTCACACTATCGGACGCGGTACTTGTGAGAGCCTC |
| | (SEQ ID NO: 20) |
| s15 | GAGGCTCTCACAGGAATCCAAGCTCGGCCTCCCGGTGTGAG |
| | AGCCTC (SEQ ID NO: 21) |
| s16 | GAGGCTCTCACACGGTTACGGTCACCCAAGCGCATTGTGAG |
| | AGCCTC (SEQ ID NO: 22) |
| s17 | GAGGCTCTCACATCCACCCTTCCGCGATGACATGGTGTGAG |
| | AGCCTC (SEQ ID NO: 23) |
| s18 | GAGGCTCTCACATCACTGATCACAGCTCTTTTTGGTGTGAG |
| | AGCCTC (SEQ ID NO: 24) |
| s19 | GAGGCTCTCACATCGCAAAAAGGGTCAGAATTCGGTGTGAG |
| | AGCCTC (SEQ ID NO: 25) |
| s20 | GAGGCTCTCACATCGCCCCATTCCCTGTTGCTCGGTGTGAG |
| | AGCCTC (SEQ ID NO: 26) |
| s21 | GAGGCTCTCACATCGCGCCTTTCCCCAGCTTTCGGTGTGAG |
| | AGCCTC (SEQ ID NO: 27) |
| s22 | GAGGCTCTCACATCGATGCCTCCTTTACTATACCGTGTGAG |
| | AGCCTC (SEQ ID NO: 28) |

TABLE 3B

| Aptamer | Nucleotide sequence |
|---|---|
| s23 | GAGGCTCTCACATCGTTCCACCCTTTCTGTTTCGGTGTGAG |
| | AGCCTC (SEQ ID NO: 29) |
| s24 | GAGGCTCTCACATCGTGGTCAGGTTGATTTGCTGGTGTGAG |
| | AGCCTC (SEQ ID NO: 30) |
| s25 | GAGGCTCTCACATCGGTGTGGCAGGTTTATTACGGTGTGAG |
| | AGCCTC (SEQ ID NO: 31) |
| s26 | GAGGCTCTCACATCGGATACACACCAACTGCTTGGTGTGAG |
| | AGCCTC (SEQ ID NO: 32) |
| s27 | GAGGCTCTCACATCGGACGACCTAAGGCAAAACGGTGTGAG |
| | AGCCTC (SEQ ID NO: 33) |
| s28 | GAGGCTCTCACACACTCCTTCATACTCCCTCGGCCTGTGAG |
| | AGCCTC (SEQ ID NO: 34) |
| s29 | GAGGCTCTCACAACTATGCGCTGGCACCTCTTGTCTGTGAG |
| | AGCCTC (SEQ ID NO: 35) |
| s30 | GAGGCTCTCACAACGCTCCCTCCCAAGTATTATGGTGTGAG |
| | AGCCTC (SEQ ID NO: 36) |
| s31 | GAGGCTCTCACATCTTCGGCTCCCTCCTCTCAGACTGTGAGA |
| | GCCTC (SEQ ID NO: 37) |
| s32 | GAGGCTCTCACATCGTTCTTTGGTGCGGTGAATGGTGTGAGA |
| | GCCTC (SEQ ID NO: 38) |
| s33 | GAGGCTCTCACATCGGGGGCGCTCTTTAATATTGGTGTGAGA |
| | GCCTC (SEQ ID NO: 39) |
| s34 | GAGGCTCTCACATAAGTGTGATCGAGCCCTCCTGGTGTGAGA |
| | GCCTC (SEQ ID NO: 40) |
| s35 | GAGGCTCTCACATTTACTCTCGCCATCGATCACGGTGTGAGA |
| | GCCTC (SEQ ID NO: 41) |
| s36 | GAGGCTCTCACAACGCGCCTCCCGTCCGAATTCGGTGTGAGA |
| | GCCTC (SEQ ID NO: 42) |
| s37 | GAGGCTCTCACACTGTCGCGCCTCTCCGGATATGGTGTGAGA |
| | GCCT (SEQ ID NO: 43) |
| s38 | GAGGCTCTCACATGCGCAGTCCCCTCACGTTACCTTGTGAGA |
| | GCCT (SEQ ID NO: 44) |
| s39 | GAGGCTCTCACAACCACGTTCCCGGCATGTCATTATGTGAGA |
| | GCCTC (SEQ ID NO: 45) |
| s40 | GAGGCTCTCACATCGCGGTAGTCCCTTTTTCGGTGTGAGAG |
| | CCTC (SEQ ID NO: 46) |
| s41 | GAGGCTCTCACACGTTCGCTGTTCGTGGTAATATGTGAGAG |
| | CCTC (SEQ ID NO: 47) |
| s42 | GAGGCTCTCACATTGTGCGATCCCTTTATACGGTGTGAGAG |
| | CCTC (SEQ ID NO: 48) |
| s43 | GAGGCTCTCACATATGCCAGCTTTCCATCACGGTGTGAGAG |
| | CCTC (SEQ ID NO: 49) |
| s44 | GAGGCTCTCACAACTCCGCGCCGACCCATTATGTGAGAGCC |
| | TC (SEQ ID NO: 50) |
| s45 | GAGGCTCTCACACGCCGGATTCCCCTGTATTTGTGAGAGCC |
| | TC (SEQ ID NO: 51) |

TABLE 3C

| Aptamer | Nucleotide sequence |
|---|---|
| s46 | GAGGCTCTCACATCTGGGGCGGTCATTAAGGTGTGAGAGCCTC |
| | (SEQ ID NO: 52) |
| s47 | GAGGCTCTCACATCGGTCCCCCTTTAAACGGTGTGAGAGCCTC |
| | (SEQ ID NO: 53) |
| s48 | GAGGCTCTCACATCGGCCTCTCCTTGTTTGGTGTGAGAGCCTC |
| | (SEQ ID NO: 54) |
| 849 | GAGGCTCTCACATCGCCCTCTCGGCACTCGGTGTGAGAGCCTC |
| | (SEQ ID NO: 55) |
| 850 | GAGGCTCTCACATCGCACAGGTTTAGTACGGTGTGAGAGCCTC |
| | (SEQ ID NO: 56) |
| s51 | GAGGCTCTCACATCAGGCTCCTCCTTATTGGTGTGAGAGCCTC |
| | (SEQ ID NO: 57) |
| 852 | GAGGCTCTCACATCTGTTGCCTCTCCGGAACTGTGAGAGCCTC |
| | (SEQ ID NO: 58) |
| s53 | GAGGCTCTCACAATCATACTCCCCGCTTTGGTGTGAGAGCCTC |
| | (SEQ ID NO: 59) |
| s54 | GAGGCTCTCACAGGGGTTCCGTAGGGAGTGGTGTGAGAGCCTC |
| | (SEQ ID NO: 60) |
| s55 | GAGGCTCTCACATTGGCACATGGCGTTACGGTGTGAGAGCCTC |
| | (SEQ ID NO: 61) |
| C1 | GAGGCTCTCACATTGGGGCGTGCTGACGGTGTGAGAGCCTC |
| | (SEQ ID NO: 62) |
| C2 | GAGGCTCTCACATTGCACTCCTTCATCGGTGTGAGAGCCTC |
| | (SEQ ID NO: 63) |
| C4 | GAGGCTCTCACATCGGGCTCCTTTAACGGTGTGAGAGCCTC |
| | (SEQ ID NO: 64) |
| C6 | GAGGCTCTCACACTTTGCTGGGGCGGACTTGTGAGAGCCTC |
| | (SEQ ID NO: 65) |
| C95 | GAGGCTCTCACATCGGGCTCCTTTATCGGTGTGAGAGCCTC |
| | (SEQ ID NO: 66) |

### Example 2-1

### Secondary Screening of Candidate Aptamers Based on Inhibitory Activity Against Cas9

A test was conducted on whether the cleavage of a plasmid comprising an EGFP(GFP) sequence by Cas9 and GFP-targeting sgRNA (sgRNA(GFPg1): sgRNA having a guide sequence designed so as to target a GFP-g1 site) is inhibited in the presence of each candidate aptamer. When the candidate aptamer lacks inhibitory activity against Cas9, the GFP sequence in the plasmid is cleaved by a complex of Cas9 and sgRNA(GFPg1). On the other hand, when the candidate aptamer has inhibitory activity against Cas9, the cleavage is suppressed. The GFP sequence contained in the plasmid is as follows.

GFP:
(SEQ ID NO: 67)
ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGG
GCGAGGGCGATGCCACCTACGGCAAGCTGACCCTGAAGTTCATCTGCACC
ACCGGCAAGCTGCCCGTGCCCTGGCCCACCCTCGTGACCACCCTGACCTA
CGGCGTGCAGTGCTTCAGCCGCTACCCCGACCACATGAAGCAGCACGACT
TCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGG
CGACACCCTGGTGAACCGCATCGAGCTGAAGGGCATCGACTTCAAGGAGG
ACGGCAACATCCTGGGGCACAAGCTGGAGTACAACTACAACAGCCACAAC
GTCTATATCATGGCCGACAAGCAGAAGAACGGCATCAAGGTGAACTTCAA
GATCCGCCACAACATCGAGGACGGCAGCGTGCAGCTCGCCGACCACTACC
AGCAGAACACCCCCATCGGCGACGGCCCCGTGCTGCTGCCCGACAACCAC
TACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCA
TGGACGAGCTGTACAAG

An amount of 0.5 pmol (final concentration: 50 nM) of Cas9 protein, 0.3 pmol of sgRNA (final concentration: 30 nM), 1 pmol of each candidate aptamer (final concentration: 100 nM), and a plasmid comprising a GFP sequence (final concentration: 3 nM) were mixed in 10 μl of a reaction buffer (20 mM HEPES pH 6.5, 100 mM NaCl, 5 mM MgCl₂, and 0.1 mM EDTA), and incubated at 37° C. for 20 minutes. Then, the cleavage of the plasmid was confirmed by 0.65% agarose gel electrophoresis.

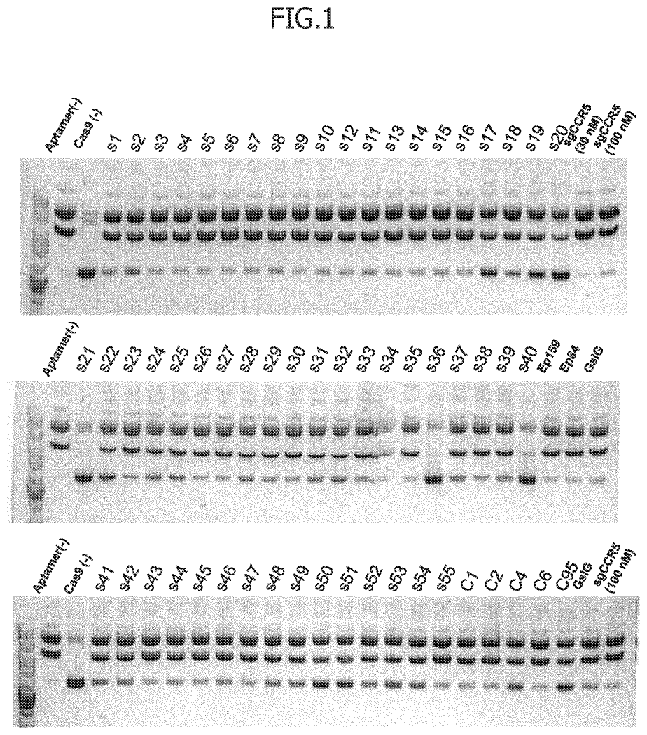

The results are shown in FIG. 1. In the drawing, “Cas9(—)” represents the results about a control sample without Cas9, i.e., the case where the plasmid was not cleaved. On the other hand, “Aptamer(—)” represents the results about a sample prepared without the addition of any candidate aptamer, i.e., the case in which the plasmid was cleaved. Two high-molecular-weight bands (open circular DNA and single-stranded DNA) resulting from the cleavage of the plasmid can be confirmed. Thus, the candidate aptamers can be evaluated as having higher inhibitory activity against Cas9 as a low-molecular-weight band is more strongly detected.

From the results of FIG. 1, s21, s36 and s40 among 60 candidate aptamers of s1 to s55, C1, C2, C4, C6, and C95 were found to have high Cas9 inhibitory activity. “Ep159”, “Ep84” and “Gs1G” were negative controls using an irrelevant nucleic acid aptamer sequence, none of which were confirmed to have Cas9 inhibitory activity. The “sgCCR5” represents the results obtained by adding sgRNA (sgRNA (CCR5)) having a guide sequence designed so as to target another sequence (CCR5 sequence) instead of the candidate aptamers. The competitive inhibitory effect of sgRNA (CCR5) with final concentrations of 30 nM and 100 nM was not observed

The sequence of the GFP-targeting sgRNA (sgRNA(GFPg1)) (Nat. Biotechnol. 2015, 33 (1): 73-80. doi:10.1038/nbt.3081.), the sequence of sgRNA (CCR5), and the sequences of irrelevant nucleic acid aptamers (Ep159, Ep84, and Gs1G) are shown below.

TABLE 4

| sgRNA | Nucleotide sequence |
|---|---|
| sgRNA | ggccucgaacuucaccucggcgguuuuagagcuagaaauag |
| (GFPg1) | caaguuaaaauaaggcuaguccguuaucaacuugaaaagug |
| | gcaccgagucggugcuuuuuuu (SEQ ID NO: 68) |
| sgRNA | gguuuugcaguuuaucaggaugguuuuagagcuagaaauag |
| (CCR5) | caaguuaaaauaaggcuaguccguuaucaacuugaaaaagu |
| | ggcaccgagucggugcuuuuuuu (SEQ ID NO: 69) |
| Aptamer | Nucleotide sequence |
| Ep159 | GAGGCTCTCACA CGCGACGAAGCTGGACA |
| | TGTGAGAGCCTC (SEQ ID NO: 70) |
| Ep84 | GAGGCTCTCACA GGAGCGACGGGATCTCA |
| | TGTGAGAGCCTC (SEQ ID NO: 71) |
| GslG | GAGGCTCTCACA |
| | GGTCCGGTACGGCACGGTCGCGAAGCGAGGTCTGGGGTGG |
| | GGAGG TGTGAGAGCCTC (SEQ ID NO: 72) |

In the notation of the nucleic acids, the upper-case characters represent DNA, and the lower-case characters represent RNA.

In Examples given below, the inhibitory activity of aptamers was evaluated by the same procedure as that in Example 2-1, unless otherwise specified.

### Example 2-2

### Revaluation of Inhibitory Activity of Candidate Aptamer

The s21, s36, and s40 confirmed to have high Cas9 inhibitory activity were reevaluated for their inhibitory activities at varying concentrations of the aptamers. Specifically, the final concentration of each candidate aptamer was set to 3 nM, 10 nM, or 30 nM, and the inhibitory activities of the aptamers were evaluated by the same procedure as that in Example 2-1.

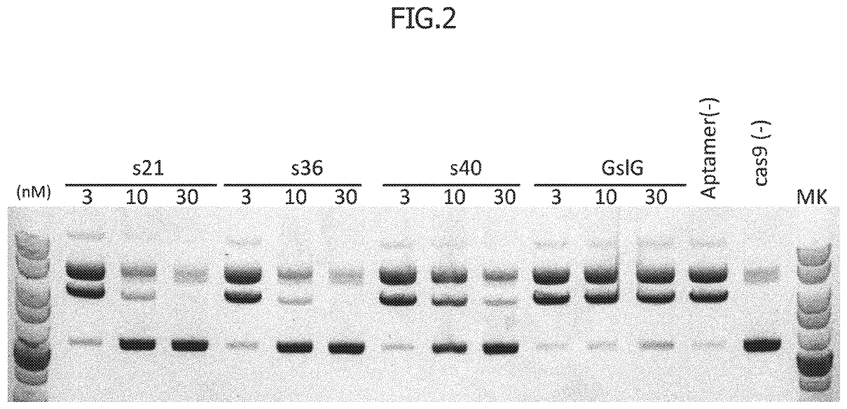

The results are shown in FIG. 2. As a result, the nucleic acid aptamers s21 and s36 were found to have high inhibitory activities against Cas9/sgRNA(GFP-g1).

### Example 3

### Analysis of Correlation Between Sequence/Structure of Nucleic Acid Aptamer and Inhibitory Activity by Mutagenesis Experiment

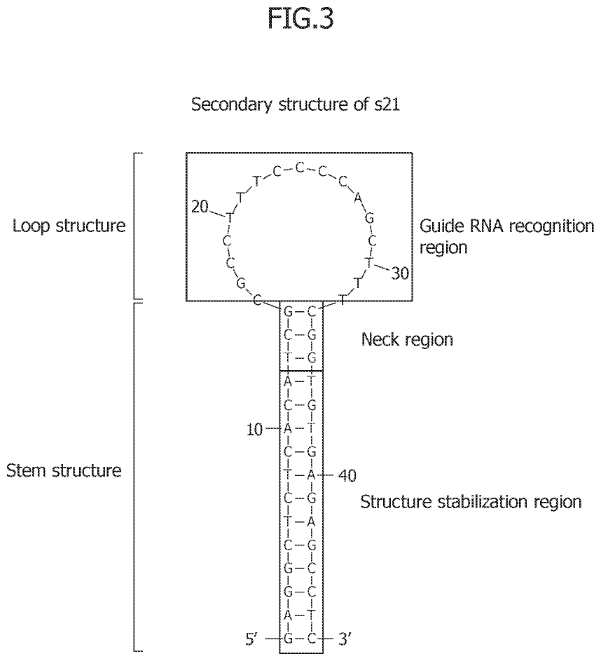

The s21 that exhibited high inhibitory activity against Cas9/sgRNA(GFP-g1) had a sequence with the 5′ end and the 3′ end complementary to each other, and therefore presumably formed a secondary structure as shown in FIG. 3. On the basis of this putative secondary structure, the nucleic acid aptamer was divided into the following three regions and subjected to mutagenesis analysis:

(1) Structure stabilization region: Forming a double-strand. Comprising PCR primer-binding sequences, and having a fixed sequence in each library derived from the aptamer.

(2) Neck region: Forming a double-strand. Being a variable region having a random sequence in the library.

(3) Guide RNA recognition region: Forming a single-stranded loop structure. Being a variable region having a random sequence in the library.

### Example 3-1

### Mutagenesis Analysis of Guide RNA Recognition Region

First, the influence of the length of the guide RNA recognition region on the inhibitory activity of the nucleic acid aptamer was examined. Truncated mutant aptamers, having a common sequence between s21 and s36 but differing in length of the guide RNA recognition region, were synthesized (s21-1, s21-2, s21-3, s21-4, s21-5, s21-6, and Tetra), and examined for their inhibitory activities against Cas9/sgRNA(GFP-g1). The sequences of the mutant aptamers are shown in Table 5. The underlined sequence (CGCC) is a sequence suggested to be important for the inhibitory activity against Cas9/sgRNA(GFP-g1) from the results of the present Example.

TABLE 5

| Aptamer | Nucleotide sequence |
|---|---|
| s21-1 | GAGGCTCTCACA TCG CGCCTTTCTTT CGG |
| | TGTGAGAGCCTC (SEQ ID NO: 73) |
| s21-2 | GAGGCTCTCACA TCG CGCCTTT CGG TGTGAGAGCCTC |
| | (SEQ ID NO: 74) |
| s21-3 | GAGGCTCTCACA TCG CGCCTT CGG TGTGAGAGCCTC |
| | (SEQ ID NO: 75) |
| s21-4 | GAGGCTCTCACA TCG CGCCTTTTTT CGG |
| | TGTGAGAGCCTC (SEQ ID NO: 76) |
| s21-5 | GAGGCTCTCACA TCG CGCCTTTTT CGG |
| | TGTGAGAGCCTC (SEQ ID NO: 77) |
| s21-6 | GAGGCTCTCACA TCG CGCCTTTT CGG |
| | TGTGAGAGCCTC (SEQ ID NO: 78) |
| Tetra | GAGGCTCTCACA TCG GTAA CGG TGTGAGAGCCTC |
| | (SEQ ID NO: 79) |

Furthermore, nucleic acid aptamers (N19-M1, N19-M2, N19-13, and N19-14: the sequences are shown in Table 6), comprising a sequence in common with s21 and s36, were selected from among the sequences obtained by the screening of the candidate aptamers, and evaluated for their inhibitory activities.

TABLE 6

| Aptamer | Nucleotide sequence |
|---|---|
| N19-M1 | GAGGCTCTCACA ACTCCGCGCCGACCCATTA |
| | TGTGAGAGCCTC (SEQ ID NO: 80) |
| N19-M2 | GAGGCTCTCACA TGGGTACGCGCCTTTGTGG |
| | TGTGAGAGCCTC (SEQ ID NO: 81) |
| N19-13 | GAGGCTCTCACA TCG CGCCCCTCATCAT CGG |
| | TGTGAGAGCCTC (SEQ ID NO: 82) |
| N19-14 | GAGGCTCTCACA TTGCCCTCTTTCAATACGG |
| | TGTGAGAGCCTC (SEQ ID NO: 83) |

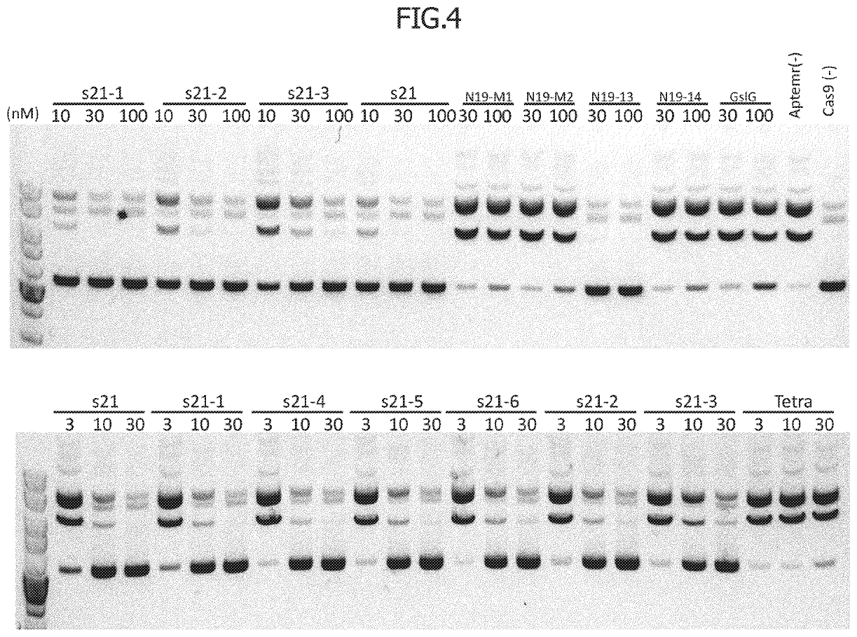

The results are shown in FIG. 4. The inhibitory activity was not much reduced in the aptamers comprising the guide RNA recognition region having a length of 7 bases or more, whereas the inhibitory activity was significantly reduced in the aptamer (s21-3) comprising the guide RNA recognition region having a length of 6 bases. On the other hand, the inhibitory activity was markedly reduced in the aptamer with the guide RNA recognition region replaced with a tetraloop sequence, known to form a stable loop structure (Tetra). Among the nucleic acid aptamers comprising a sequence in common with s21 and s36, only N19-13, which had the neck region and CGCC as the sequence of the first (5′-terminal) four bases in the guide RNA recognition region, exhibited inhibitory activity, inferring that the first CGCC in the guide RNA recognition region is important for the inhibitory activity against Cas9/sgRNA(GFP-g1).

In order to further analyze the influence of the position of CGCC in the guide RNA recognition region on the inhibitory activity, candidate aptamers comprising the guide RNA recognition region starting with CGCC (N23-meme1 and N23-meme2), candidate aptamers containing CGCC at any position of the guide RNA recognition region (N23-m1, N23-m2, and N23-m3), and candidate aptamers having a mutation in the neck region of s21 and s36 (s21-mut1 and s36-mut1) were selected from among the sequences obtained by the screening of the candidate aptamers, and examined for their inhibitory activities against Cas9/sgRNA(GFP-g1). The sequences of these candidate aptamers are shown in Table 7.

TABLE 7

| | |
|---|---|
| N23- | GAGGCTCTCACA TCG CGCCTCCCTGTAAAATT CGG |
| meme1 | TGTGAGAGCCTC (SEQ ID NO: 84) |
| N23- | GAGGCTCTCACA TCG CGCCTCTCCGCAACATA CGG |
| meme2 | TGTGAGAGCCTC (SEQ ID NO: 85) |
| N23-m1 | GAGGCTCTCACA TCG TGACCTACTCGCGCCGT ATG |
| | TGTGAGAGCCTC (SEQ ID NO: 86) |
| N23-m2 | GAGGCTCTCACA CTG TCGCGCCTCTCCGGATA TGG |
| | TGTGAGAGCCTC (SEQ ID NO: 87) |
| N23-m3 | GAGGCTCTCACA CCCTCCACACGCGCCTGGTCCA |
| | TGTGAGAGCCTC (SEQ ID NO: 88) |
| s21-mut1 | GAGGCTCTCACA TCTCGCCTTTCCCCAGCTTT CGG |
| | TGTGAGAGCCTC (SEQ ID NO: 89) |
| s36-mut1 | GAGGCTCTCACA ACG CGCCTCCCGTCCGAATT AGG |
| | TGTGAGAGCCTC (SEQ ID NO: 90) |

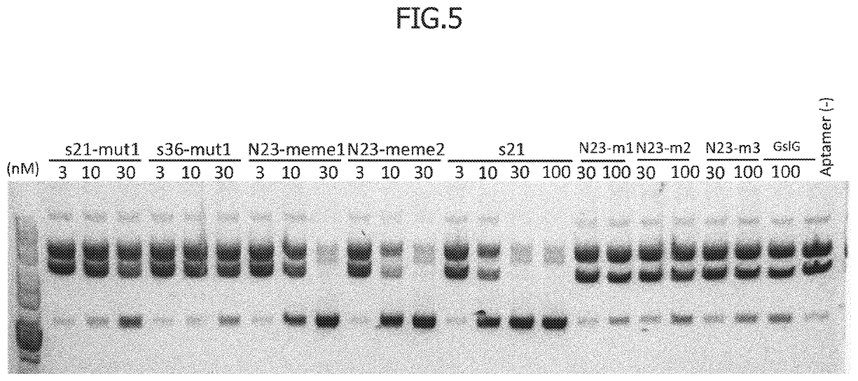

The results are shown in FIG. 5. Both the candidate aptamers (N23-meme1 and N23-meme2) comprising the guide RNA recognition region starting with CGCC exhibited inhibitory activity at similar levels as that of s21. On the other hand, all of the candidate aptamers (N23-m1, N23-m2, and N23-m3), containing CGCC at any of the other positions of the guide RNA recognition region, and the candidate aptamers (s21-mut1 and s36-mut1), having a mutation in the neck region of s21 or s36, exhibited only weak inhibitory activity compared with s21.

Furthermore, nucleic acids having a point mutation introduced in the guide RNA recognition region of s21-2 were prepared (s21-21m1, s21-21m2, s21-21m3, s21-21m4, s21-21m5, s21-21m6, and s21-21m7), and evaluated for their inhibitory activities. The sequences of these nucleic acids are shown in Table 8 (site of mutation underlined).

TABLE 8

| Aptamer | Nucleotide sequence |
|---|---|
| s21-2lm1 | GAGGCTCTCACA TCG GGCCTTT CGG |
| | TGTGAGAGCCTC (SEQ ID NO: 91) |
| s21-2lm2 | GAGGCTCTCACA TCG CCCCTTT CGG |
| | TGTGAGAGCCTC (SEQ ID NO: 92) |
| s21-2lm3 | GAGGCTCTCACA TCG CGGCTTT CGG |
| | TGTGAGAGCCTC (SEQ ID NO: 93) |
| s21-2lm4 | GAGGCTCTCACA TCG CGCGTTT CGG |
| | TGTGAGAGCCTC (SEQ ID NO: 94) |
| s21-2lm5 | GAGGCTCTCACA TCG CGCCATT CGG |
| | TGTGAGAGCCTC (SEQ ID NO: 95) |
| s21-2lm6 | GAGGCTCTCACA TCG CGCCTAT CGG |
| | TGTGAGAGCCTC (SEQ ID NO: 96) |
| s21-2lm7 | GAGGCTCTCACA TCG CGCCTTA CGG |
| | TGTGAGAGCCTC (SEQ ID NO: 97) |

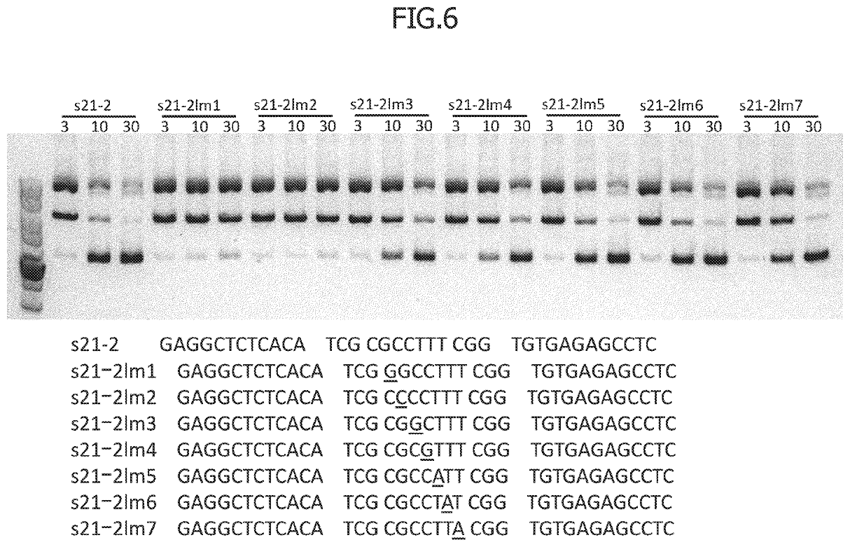

The results are shown in FIG. 6. The inhibitory activity disappeared critically in the aptamer (s21-21m1) having a point mutation introduced (C→G) in the first base and the aptamer (s21-21m2) having a point mutation introduced (G→C) in the second base in the guide RNA recognition region (7 bases long). The inhibitory activity was also significantly reduced in the aptamer (s21-21m3) having a point mutation introduced (C→G) in the third base and the aptamer (s21-21m4) having a point mutation introduced (C→G) in the fourth base. On the other hand, point mutagenesis in the 5th to 7th bases did not have large influence on the inhibitory activity (s21-21m5, s21-21m6, and s21-21m7). From these results, it was confirmed that the first CGCC sequence of the guide RNA recognition region is important for the inhibitory activity against Cas9/sgRNA(GFP-g1), and the other sequence is not particularly important.

### Example 3-2

### Mutagenesis Analysis of Structure Stabilization Region and Neck Region

Next, the correlation between the sequence/structure of the structure stabilization region and the inhibitory activity of the aptamer was examined. In order to first examine the influence of the length of the structure stabilization region, nucleic acids comprising the structure stabilization region that was 11 base pairs long (s21-2e), 10 base pairs long (s21-2f), 9 base pairs long (s21-2g), 8 base pairs long (s21-2a), 6 base pairs long (s21-2b), 3 base pairs long (s21-2c), or 0 base pairs long (s21-2d) were prepared on the basis of s21-2 (structure stabilization region: 12 base pairs long), and examined for their inhibitory activities against Cas9/sgRNA (EGFP-g1). The sequences of these nucleic acids are shown in Table 9.

TABLE 9

| Aptamer | Nucleotide sequence |
|---|---|
| s21-2e | AGGCTCTCACA TCG CGCCTTT CGG TGTGAGAGCCT |
| | (SEQ ID NO: 98) |
| s21-2f | GGCTCTCACA TCG CGCCTTT CGG TGTGAGAGCC |
| | (SEQ ID NO: 99) |
| s21-2g | GCTCTCACA TCG CGCCTTT CGG TGTGAGAGC |
| | (SEQ ID NO: 100) |
| s21-2a | CTCTCACA TCG CGCCTTT CGG TGTGAGAG |
| | (SEQ ID NO: 101) |
| s21-2b | CTCACA TCG CGCCTTT CGG TGTGAG |
| | (SEQ ID NO: 102) |
| s21-2c | ACA TCG CGCCTTT CGG TGT (SEQ ID NO: 103) |
| s21-2d | TCG CGCCTTT CGG (SEQ ID NO: 104) |

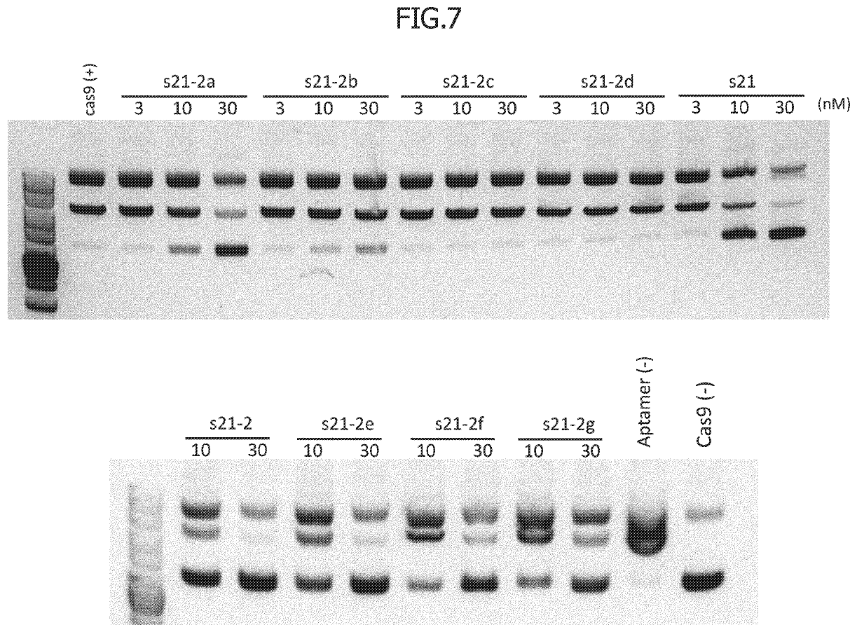

The results are shown in FIG. 7. The inhibitory activity was gradually reduced with decrease in the length of the structure stabilization region to the length of 8 base pairs, whereas the inhibitory activity was rapidly lost when the length was 6 base pairs or fewer.

Next, in order to confirm the importance of the nucleotide sequences of the structure stabilization region and the neck region, nucleic acids having a sequence substituted by every 2 base pairs from the free end, whereas retaining the stem structure were prepared (s21-2m1 to s21-2m7) on the basis of s21-2, and examined for their inhibitory activities against Cas9/sgRNA (EGFP-g1). The sequences of these nucleic acids are shown in Table 10 (site of mutation underlined).

TABLE 10

| Aptamer | Nucleotide sequence |
|---|---|
| s21-2m1 | CTGGCTCTCACA TCG CGCCTTT CGG TGTGAGAGCCAG |
| | (SEQ ID NO: 105) |
| s21-2m2 | GACCCTCTCACA TCG CGCCTTT CGG TGTGAGAGGGTC |
| | (SEQ ID NO: 106) |
| s21-2m3 | GAGGGACTCACA TCG CGCCTTT CGG TGTGAGTCCCTC |
| | (SEQ ID NO: 107) |
| s21-2m4 | GAGGCTGACACA TCG CGCCTTT CGG TGTGTCACCTC |
| | (SEQ ID NO: 108) |
| s21-2m5 | GAGGCTCTGTCA TCG CGCCTTT CGG TGACAGAGCCTC |
| | (SEQ ID NO: 109) |
| s21-2m6 | GAGGCTCTCAGT TCG CGCCTTT CGG ACTGAGAGCCTC |
| | (SEQ ID NO: 110) |
| s21-2m7 | GAGGCTCTCACA TGC CGCCTTT GCG TGTGAGAGCCTC |
| | (SEQ ID NO: 111) |

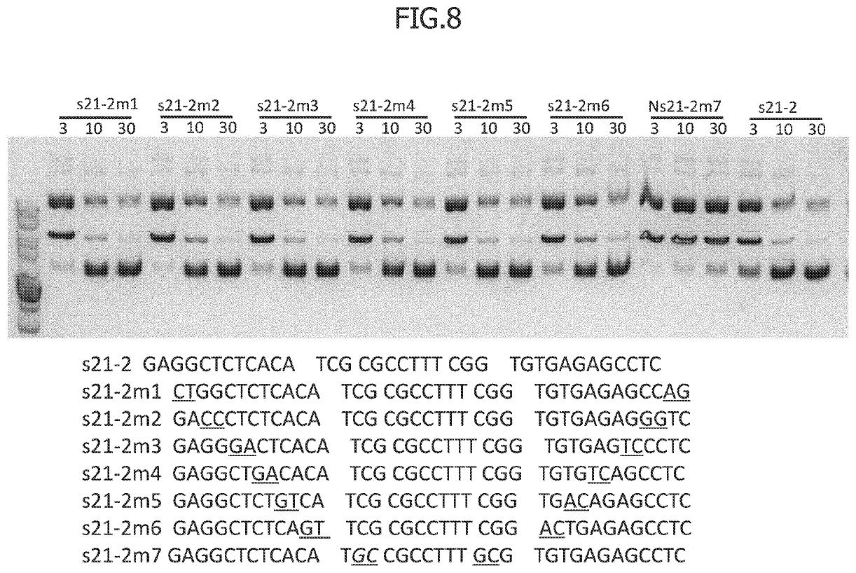

The results are shown in FIG. 8. The nucleic acids (s21-2m1 to s21-2m6), having a mutation introduced in the structure stabilization region, all exhibited inhibitory activity equivalent to that of s21-2, demonstrating that the inhibitory activity of the aptamer does not depend on the sequence of the structure stabilization region. On the other hand, the nucleic acid (s21-2m7), having a mutation introduced in the neck region, largely lost inhibitory activity, suggesting that the nucleotide sequence of the neck region is important for the inhibitory activity of the aptamer.

### Example 3-3

### Mutagenesis Analysis of Neck Region

Next, nucleic acid sequences having various mutations introduced in the neck region were prepared (s21-2mm1 to s21-2mm11), and examined for the detailed correlation between the sequence or structure of the neck region and the inhibitory activity of the aptamer. These nucleic acid sequences are shown in Table 11 (sites of mutations underlined).

TABLE 11

| Aptamer | Nucleotide sequence |
|---|---|
| s21-2mm1 | GAGGCTCTCACA ACG CGCCTTT CGG TGTGAGAGCCTC |
| | (SEQ ID NO: 112) |
| s21-2mm2 | GAGGCTCTCACA GCG CGCCTTT CGT TGTGAGAGCCTC |
| | (SEQ ID NO: 113) |
| s21-2mm3 | GAGGCTCTCACA CCG CGCCTTT CGG TGTGAGAGCCTC |
| | (SEQ ID NO: 114) |
| 821-2mm4 | GAGGCTCTCACA TCG CGCCTTT TGG TGTGAGAGCCTC |
| | (SEQ ID NO: 115) |
| s21-2mm5 | GAGGCTCTCACA TCA CGCCTTT TGG TGTGAGAGCCTC |
| | (SEQ ID NO: 116) |
| s21-2mm6 | GAGGCTCTCACA TCC CGCCTTT GGG TGTGAGAGCCTC |
| | (SEQ ID NO: 117) |
| s21-2mm7 | GAGGCTCTCACA TCA CGCCTTT CGG TGTGAGAGCCTC |
| | (SEQ ID NO: 118) |
| s21-2mm8 | GAGGCTCTCACA TGG CGCCTTT CCG TGTGAGAGCCTC |
| | (SEQ ID NO: 119) |
| s21-2mm9 | GAGGCTCTCACA TTG CGCCTTT CGG TGTGAGAGCCTC |
| | (SEQ ID NO: 120) |
| s21-2mm10 | GAGGCTCTCACA TAG CGCCTTT CGG TGTGAGAGCCTC |
| | (SEQ ID NO: 121) |
| s21-2mm11 | GAGGCTCTCACA TCCA CGCCTTT TGG TGTGAGAGCCTC |
| | (SEQ ID NO: 122) |

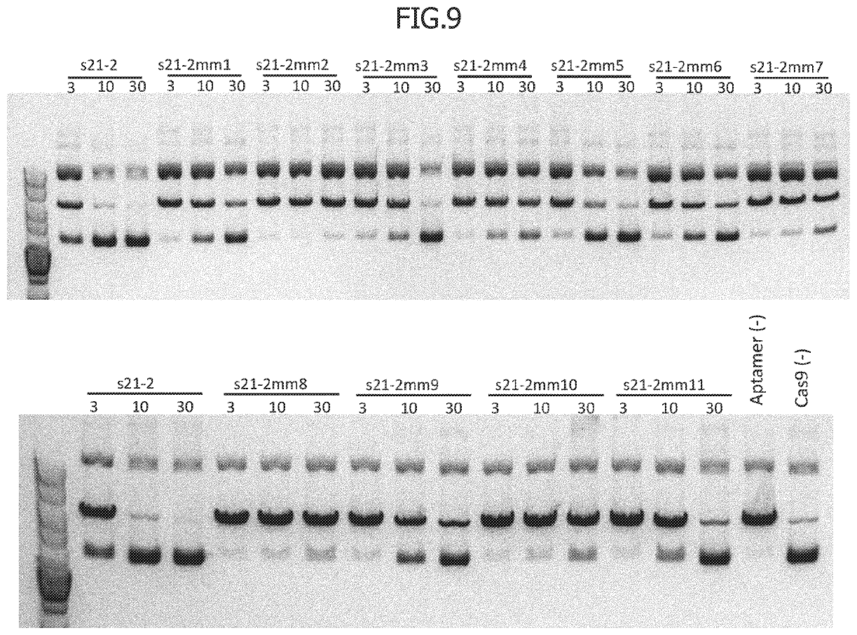

The results are shown in FIG. 9. It was found that: the types of the bases in the third base pair in the neck region, counted from a base pair adjacent to the structure stabilization region, do not influence the inhibitory activity of the aptamer as long as the base pair is formed; as for the first and second base pairs, the inhibitory activity is higher in the order of TC>CC>TT of the sequence of a neck moiety (in the present Example, referred to as a “sense sequence”) adjacent to the upstream CGCC sequence of the guide RNA recognition region; the sequence of a neck moiety having a complementary strand thereof (in the present Example, referred to as an “antisense sequence”) needs to be GG; and the insertion of a bulge to the sense sequence is acceptable (s21-2mm11).

### Example 3-4

### Inhibitory Activity of Phosphorothioated Nucleic Acid Aptamer

For in vivo use of a nucleic acid, the oxygen atom of the phosphate group of the nucleic acid is often substituted with a sulfur atom (phosphorothioate modification) in order to enhance stability. Accordingly, study was conducted to determine whether or not the phosphorothioate modification (hereinafter, referred to as “thiolation”) would influence the inhibitory activity of the aptamer. The sequences of prepared thiolated nucleic acids are shown in Table 12 (thiolated bases indicated by \hat{ }).

TABLE 12

| Aptamer | Nucleotide sequence |
|---|---|
| s21- | G\hat{ }A\hat{ }G\hat{ }GCTCTCACA TCG CGCCTTT CGG TGTGAGAG |
| ls2s1 | C\hat{ }C\hat{ }T\hat{ }C (SEQ ID NO: 123) |
| s21- | GAGG\hat{ }C\hat{ }T\hat{ }CTCACA TCG CGCCTTT CGG TGTGA |
| ls2s2 | G\hat{ }A\hat{ }G\hat{ }CCTC (SEQ ID NO: 124) |
| s21- | GAGGCTC\hat{ }T\hat{ }C\hat{ }ACA TCG CGCCTTT CGG TGT\hat{ }G\hat{ }A\hat{ }G |
| ls2s3 | AGCCTC (SEQ ID NO: 125) |
| s21- | GAGGCTCTCA\hat{ }C\hat{ }A\hat{ } TCG CGCCTTT CGG \hat{ }T\hat{ }G\hat{ }TGAG |
| ls2s4 | AGCCTC (SEQ ID NO: 126) |
| s21- | GAGGCTCTCACA T\hat{ }C\hat{ }G\hat{ } CGCCTTT \hat{ }C\hat{ }G\hat{ }GTGTGAGA |
| ls2s5 | GCCTC (SEQ ID NO: 127) |
| s21- | GAGGCTCTCACA TCG C\hat{ }G\hat{ }C\hat{ }C\hat{ }T\hat{ }T\hat{ }T CGG TGTGAG |
| ls2s6 | AGCCTC (SEQ ID NO: 128) |
| s21- | G\hat{ }A\hat{ }G\hat{ }G\hat{ }C\hat{ }T\hat{ }C\hat{ }T\hat{ }C\hat{ }A\hat{ }C\hat{ }A\hat{ } \hat{ }T\hat{ }C\hat{ }G\hat{ }C\hat{ }G\hat{ }C\hat{ }C\hat{ } |
| ls2s | T\hat{ }T\hat{ }T\hat{ }C\hat{ }G\hat{ }G\hat{ } T\hat{ }G\hat{ }T\hat{ }G\hat{ }A\hat{ }G\hat{ }A\hat{ }G\hat{ }C\hat{ }C\hat{ }T\hat{ }C |
| all | (SEQ ID NO: 129) |

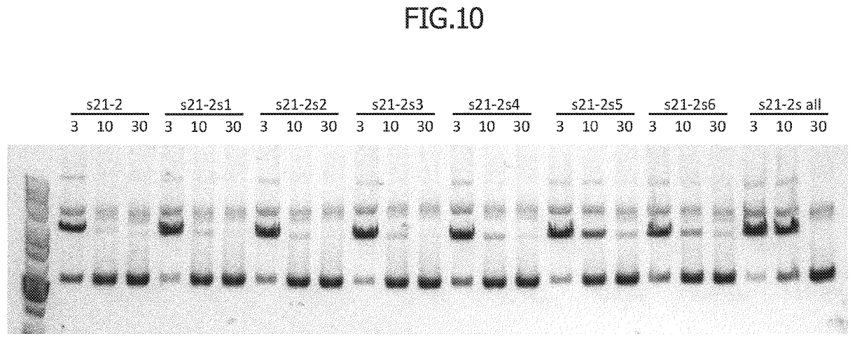

The results are shown in FIG. 10. All the nucleic acids (s21-1s2s1 to s21-1s2s5), in which 3 bases each of the sense sequence and the antisense sequence in the structure stabilization region and/or the neck region of s21-2 were thiolated, and the nucleic acid (s21-1s2s6), in which all the bases of the guide RNA recognition region were thiolated, had inhibitory activity, and their inhibitory activities was hardly reduced as compared with s21-2. On the other hand, the inhibitory activity of the nucleic acid (s21-1s2s all), in which all the bases of the entire region of s21-2 were thiolated, was significantly reduced.

### Example 4

### Analysis of Interaction Between Cas9 and Aptamer by Means of Gel Shift Assay

The interaction between Cas9 protein and each nucleic acid aptamer having inhibitory activity was analyzed by gel shift assay. The detailed experimental conditions are as follows.

Amounts of 2 pmol of Cas9 protein and 20 pmol of each nucleic acid aptamer (s21, s21-2, s21-mm2, tetra, and St2-1SA (aptamer against streptavidin)) were incubated at normal temperature for 30 minutes in a binding buffer (20 mM Tris/HCl pH 8.0, 250 mM NaCl, 1 mM MgCl₂, 2.5% glycerol, 0.05% Tween-20, and 0.05 mg/ml tRNA). Then, the obtained product was electrophoresed on 8% acrylamide gel, and the gel was stained with SYBR Gold (Thermo Fisher Scientific Inc.), followed by analysis with Bio-Rad ChemiDoc XRS image analysis system (Bio-Rad Laboratories, Inc.). The sequence of St2-1SA is as described below (Table 13).

TABLE 13

| Aptamer | Nucleotide sequence |
|---|---|
| St2-1SA | ATTGACCGCTGTGTGACGCAACACTCAAT |
| | (SEQ ID NO: 130) |

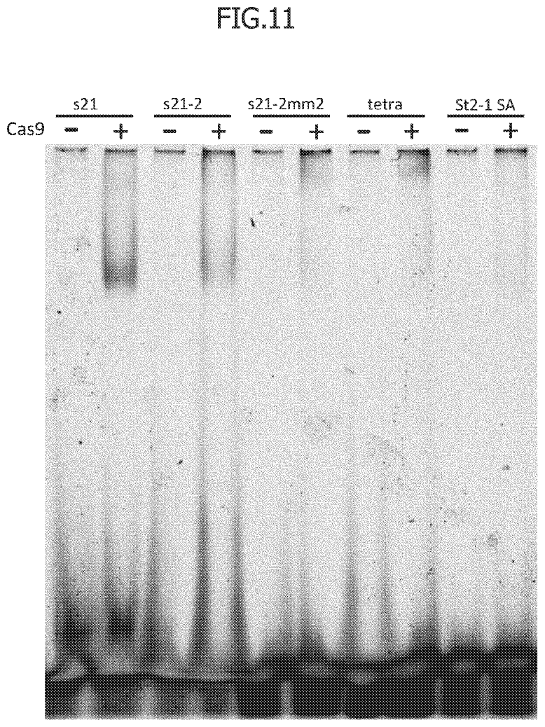

The results are shown in FIG. 11. A band indicating a complex with the Cas9 protein was seen in the upper portion of the gel for the samples using s21 and s21-2, having high Cas9 inhibitory activity. On the other hand, the band indicating a complex with the Cas9 protein was not seen for the samples using s21-2mm2, Tetra, and St2-1SA (negative control), having low Cas9 inhibitory activity.

### Example 5

### Analysis of Physicochemical Interaction between Cas9 Protein and Nucleic Acid Aptamer by means of Surface Plasmon Resonance Analysis

In order to calculate the dissociation constant (Kd) between Cas9 protein and each nucleic acid aptamer, surface plasmon resonance analysis was conducted. The detailed experimental approach was as follows.

5′-Biotinylated s21 was prepared and was immobilized on a streptavidin sensor chip (GE Healthcare Japan Corp.). Then, different concentrations (2.5 nM, 5 nM, 10 nM, and 20 nM) of Cas9 protein were loaded in a running buffer (20 mM Tris/HCl pH 8.0, 150 mM NaCl, 1 mM MgCl₂, and 0.005% Tween-20) to obtain sensorgrams. Kd was calculated with Biacore evaluation software package (GE Healthcare Japan Corp., Ver 2.0). Tetra was used as a control to conduct a similar test.

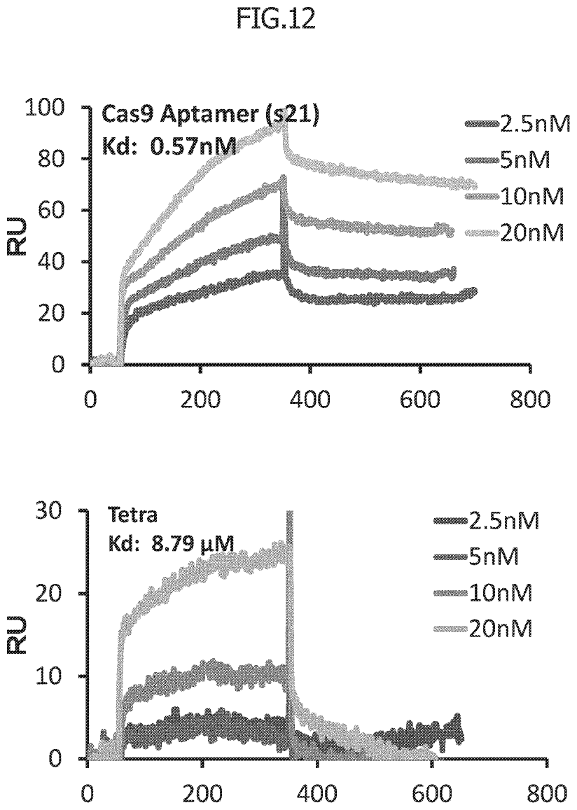

The results are shown in FIG. 12. The Kd between s21 and the Cas9 protein was 0.57 nM, demonstrating that s21 has very high ability to bind to Cas9. On the other hand, the Kd between Tetra having weak inhibitory activity and the Cas9 protein was 8.79 μM.

### Example 6

### Design of Stem-Flap-Type Aptamer

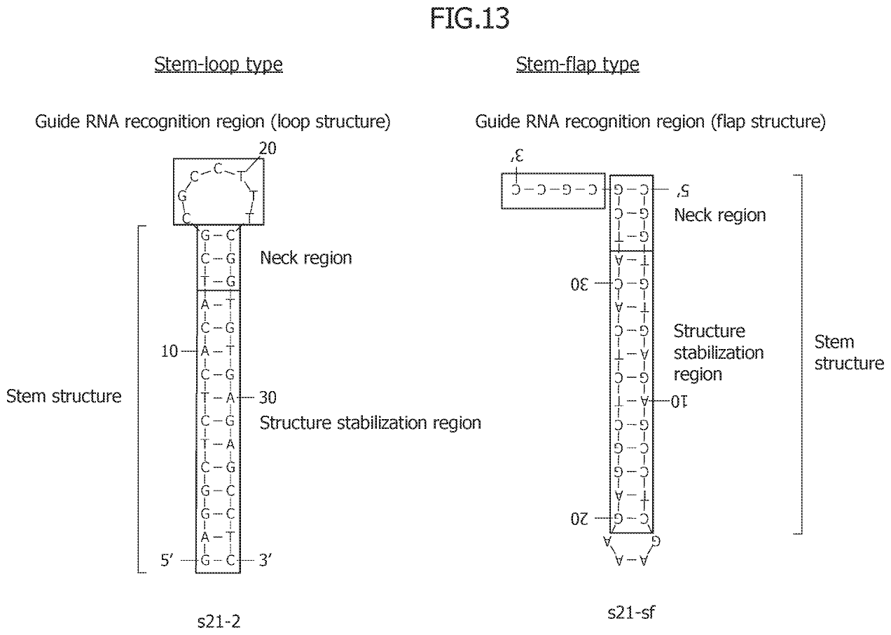

Since the first four bases of the guide RNA recognition region are important for the inhibitory activity of the aptamer against Cas9, a stem-flap-type aptamer comprising the guide RNA recognition region including only the first four bases (CGCC) was prepared (s21-sf) as shown in FIG. 13, and examined for its inhibitory activity. The sequence of s21-sf is shown below (Table 14).

TABLE 14

| Aptamer | Nucleotide sequence |
|---|---|
| s21-sf | CGG TGTGAGAGCCTC GAGG GAGGCTCTCACA TCG |
| | CGCC (SEQ ID NO: 131) |

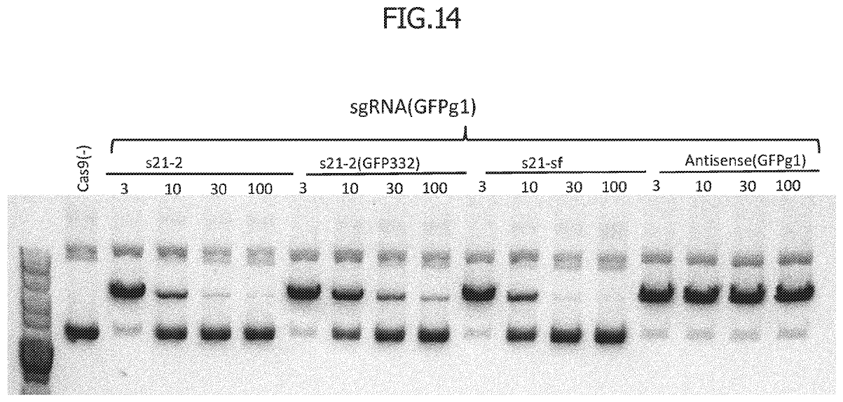

The results are shown in FIG. 14. The aptamer (s21-sf), formed the newly designed stem-flap structure, was confirmed to have inhibitory activity substantially equivalent to that of the aptamer (s21-2) that formed a stem-loop structure.

### Example 7-1

### sgRNA Sequence Specificity of Aptamer against Cas9

Since s21-2 exhibited high inhibitory activity against Cas9/sgRNA(GFPg1), study was conducted on whether s21-2 could have similar inhibitory activity against a Cas9/sgRNA complex targeting other sites on the GFP sequence. In the subsequent experiments, a complex comprising crRNA and tracrRNA annealed with each other (crRNA:tracrRNA) was used instead of sgRNA. The crRNA and the tracrRNA used were purchased from FASMAC or Integrated Device Technology, Inc. (IDT). The sequence of the crRNA is shown in Table 15 (guide sequence in crRNA underlined).

TABLE 15

| crRNA | Nucleotide sequence |
|---|---|
| crRNA | ggccucgaacuucaccucggcg guuuuagagcuaugcuguu |
| (GFPg1) | ug (SEQ ID NO: 132) |
| crRNA | caacuacaagacccgcgccg guuuuagagcuaugcuguuug |
| (GFP332) | (SEQ ID NO: 133) |
| crRNA | cgaugcccuucagcucgaug guuuuagagcuaugcuguuug |
| (GPF373) | (SEQ ID NO: 134) |
| crRNA | caugccgagagugaucccgg guuuuagagcuaugcuguuug |
| (GFP686) | (SEQ ID NO: 135) |

Three crRNAs targeting different sites in GFP were each annealed with tracrRNA to form crRNA:tracrRNA complexes. Then, s21-2 and Cas9 protein were added thereto at the same time. The inhibitory activity of s21-2 was examined. The crRNA:tracrRNA used had a final concentration of 30 nM, and Cas9 had a final concentration of 50 nM. These final concentrations were the same as those in Example 2-1. The aptamer concentrations are shown in the drawing.

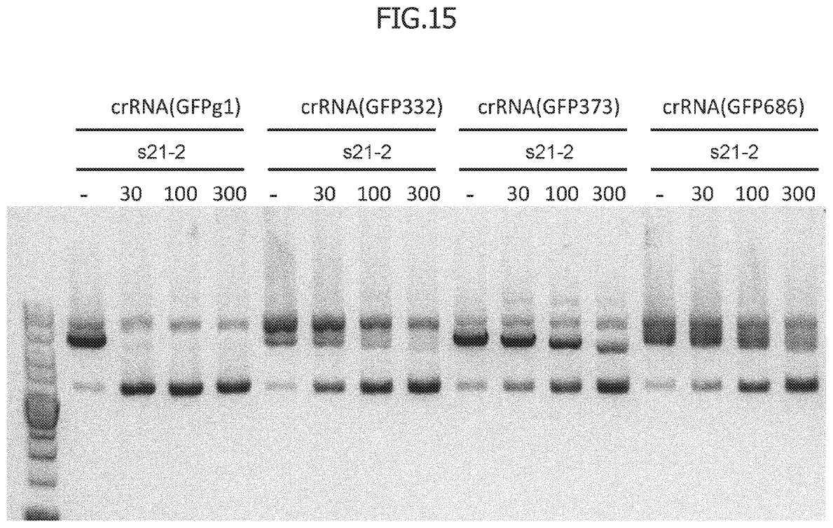

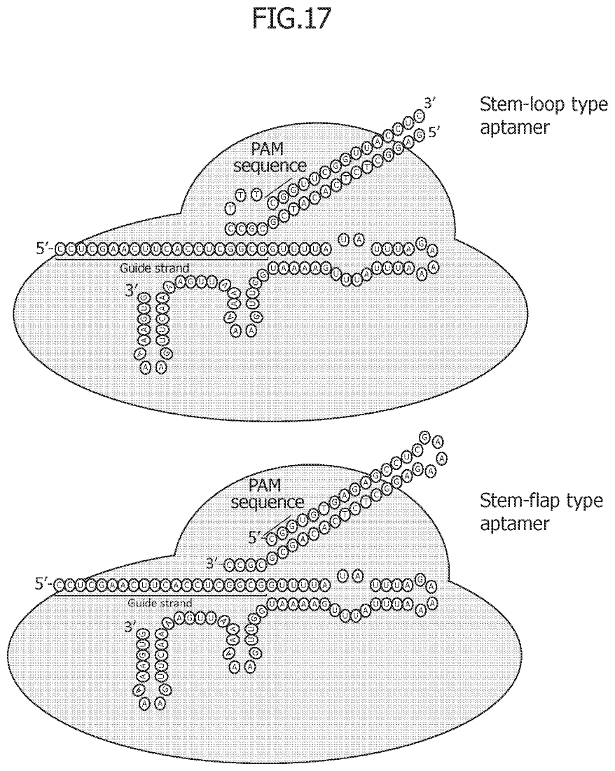

The results are shown in FIG. 15. The s21-2 exhibited high inhibitory activity against Cas9/crRNA:tracrRNA(GFPg1), targeting the same site as that of Cas9/sgRNA(GFPg1), but exhibited only weak inhibitory activity against the Cas9/crRNA:tracrRNA complexes targeting other sites (GFP332, GFP373, and GFP686). These results suggest that s21-2 might interact with crRNA(GFPg1). Referring to the sequence of s21-2, the first four bases CGCC, which are important for the inhibitory activity, in the guide RNA recognition region are complementary to the 3′-terminal four bases GGCG of the guide sequence in crRNA(GFPg1), and these bases seemed to form a double strand. The antisense sequence of the neck region was the same as the PAM sequence (NGG). In addition, discussion based on the crystal structure of Cas9/sgRNA suggested that s21-2 and s21-sf are each bound to the Cas9/sgRNA complex in a manner as shown in the schematic diagram of FIG. 17.

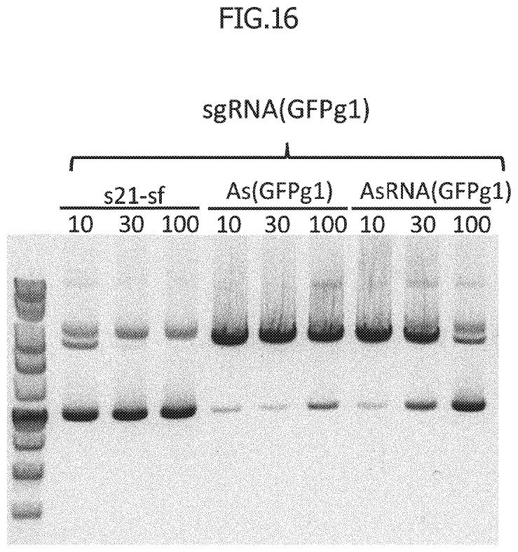

Antisense DNA (As(GFPg1)) and antisense RNA (AsRNA(GFPg1)) against the guide sequence in sgRNA(GFPg1) were evaluated for their inhibitory activities according to an existing approach (antisense method). As a result, the inhibitory activity was hardly seen (FIG. 16). The sequences of As(GFPg1) and AsRNA(GFPg1) are shown in Table 16.

TABLE 16

| Antisense | |
|---|---|
| oligonucleotide | Nucleotide sequence |
| As(GFPg1) | CGCCGAGGTGAAGTTCGAGGCC |
| | (SEQ ID NO: 136) |
| AsRNA(GFPg1) | cgccgaggugaaguucgaggcc |
| | (SEQ ID NO: 137) |

### Example 7-2

### Design of Aptamer Based on Mechanism of Specificity

Stem-flap-type aptamers (sf(GFP332) and sf(GFP373)) and stem-loop-type aptamers (s21-2(GFP332) and s21-2(GFP373)) were designed for Cas9/crRNA(GFP332) and Cas9/crRNA(GPF373), according to the binding models shown in FIG. 17, and their inhibitory activities was measured. The sequences are shown in Tables 17 and 18 (sequences probably forming a double strand with the guide sequence in crRNA are underlined).

TABLE 17

| Aptamer | Nucleotide sequence |
|---|---|
| sf(GFP332) | CGG TGTGAGAGCCTC GAGG GAGGCTCTCACA TCG |
| | CGGC (SEQ ID NO: 138) |
| sf(GFP373) | CGG TGTGAGAGCCTC GAGG GAGGCTCTCACA TCG |
| | CATC (SEQ ID NO: 139) |

TABLE 18

| Aptamer | Nucleotide sequence |
|---|---|
| s21-2(GFP332) | GAGGCTCTCACA TCG CGGCTTT CGG TGTGAG |
| | AGCCTC (SEQ ID NO: 140) |
| s21-2(GFP373) | GAGGCTCTCACA TCG CATCTTT CGG TGTGAG |
| | AGCCTC (SEQ ID NO: 141) |
| s21-A(GFPg1) | GAGGCTCTCACA TCG CGCCAAAAAA |
| | AAAAAAA CGG TGTGAGAGCCTC |
| | (SEQ ID NO: 142) |
| s21-A(GFP332) | GAGGCTCTCACA TCG CGGCAAAAAAAAAAAAA |
| | CGG TGTGAGAGCCTC (SEQ ID NO: 143) |
| s21-A(GFP373) | GAGGCTCTCACA TCG CATCAAAAAAAAAAAAA |
| | CGG TGTGAGAGCCTC (SEQ ID NO: 144) |
| As(GFP332) | CGGCGCGGGTCTTGTAGTTG |
| | (SEQ ID NO: 145) |
| As(GFP373) | CATCGAGCTGAAGGGCATCG |
| | (SEQ ID NO: 146) |

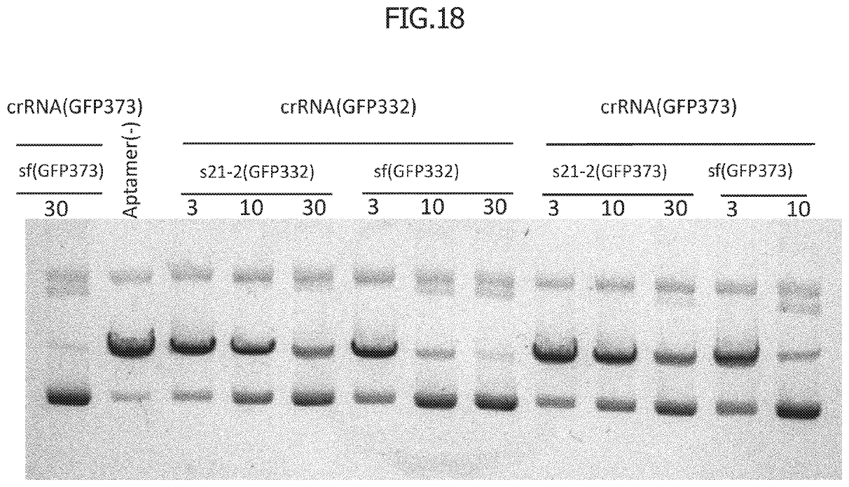

The results are shown in FIG. 18. The stem-flap-type aptamers (sf(GFP332) and sf(GFP373)) exhibited high inhibitory activity (equivalent to that of the s21 aptamer against Cas9/sgRNA(GFPg1)) against both Cas9/crRNA(GFP332) and Cas9/crRNA(GPF373). These results strongly suggest the binding manner expected by the present inventors.

The stem-loop-type aptamers against Cas9/crRNA(GFP332) or Cas9/crRNA(GPF373) (s21-2(GFP332) and s21-2(GFP373)) exhibited slightly lower inhibitory activity than that of their stem-flap-type aptamer counterparts. This is probably because a sufficient double strand with the guide strand in crRNA was not formed due to a short loop length (7 bases).

Accordingly, stem-loop-type aptamers having a loop length extended to 17 bases, all of which were constituted by A except for the 4 bases considered to form a double-strand with the guide sequence were designed (s21-A(GFP332) and s21-A(GFP373)) (the sequences are shown in Table 18 above), and their inhibitory activities against Cas9/crRNA(GFP332) or Cas9/crRNA(GPF373) were measured.

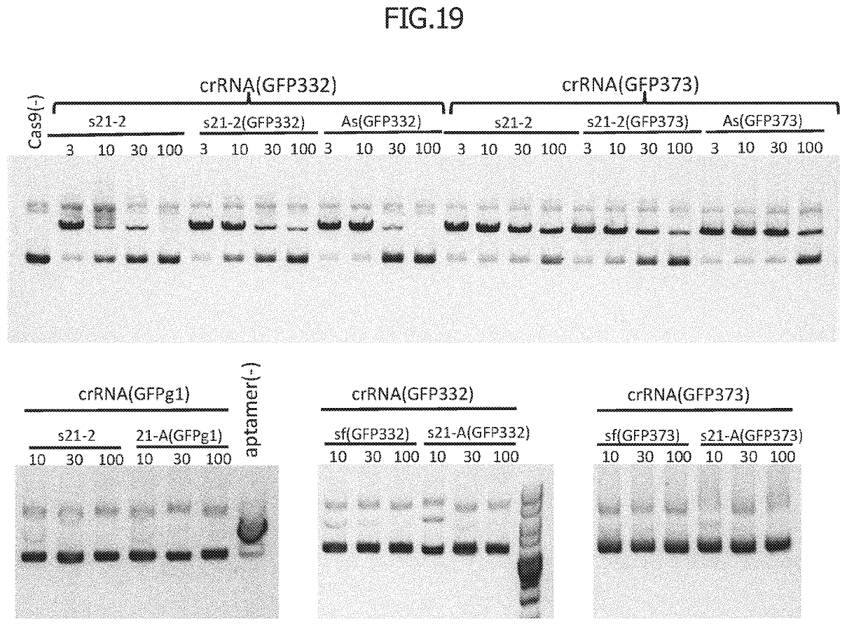

The results are shown in FIG. 19. Both s21-A(GFP332) and s21-A(GFP373) exhibited inhibitory activity equivalent to that of their stem-flap-type aptamer counterparts. These results indicated that the extension of the loop length can restore the inhibitory activity. The antisense DNA (As(GFP332) and As(GFP373)) against the guide sequence exhibited only lower inhibitory activities as compared with the aptamers described above.

### Example 8

### Experiment on Structural Parameter of Stem-Flap-Type Aptamer

In order to examine the specificity of each stem-flap-type aptamer for the guide sequence, aptamers targeting the GFPg1 site for Cas9/crRNA(GFP332) and Cas9/crRNA(GPF373) respectively (s21-2 and s21-sf), a negative control aptamer having no flap structure (sf-0), a stem-flap-type aptamer targeting the GFP332 site (sf(GFP332)), and a stem-flap-type aptamer targeting the GFP373 site (sf(GFP373)) were evaluated for their inhibitory activities. The sequence of sf-0 is shown in the table.

TABLE 19

| Aptamer | Nucleotide sequence |
|---|---|
| sf-0 | CGG TGTGAGAGCCTC GAGG GAGGCTCTCACA TCG |
| | (SEQ ID NO: 147) |

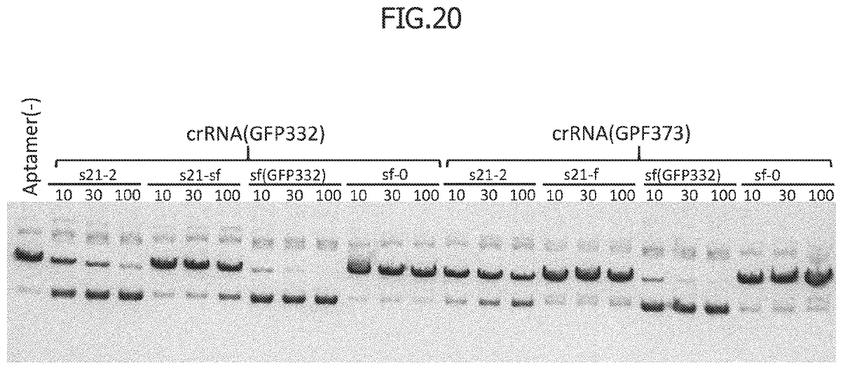

The results are shown in FIG. 20. The sf(GFP332) and sf(GFP373) exhibited very high inhibitory activity against Cas9/crRNA(GFP332) and Cas9/crRNA(GPF373), respectively. In contrast, s21-sf (stem-flap-type aptamer targeting the GFPg1 site) hardly exhibited inhibitory activity against either of Cas9/crRNA(GFP332) and Cas9/crRNA(GPF373). From these results, the stem-flap-type aptamers were found to have high sequence specificity. On the other hand, s21-2 (stem-loop-type aptamer targeting the GFPg1 site) exhibited slight inhibitory activity against both Cas9/crRNA(GFP332) and Cas9/crRNA(GPF373), suggesting slightly low sequence specificity.

Next, the relationship between the flap length and the inhibitory activity of each stem-flap-type aptamer was examined. Stem-flap-type aptamers having varying lengths of the flap structure (sf-0, sf-2(GFPg1), sf-3(GFPg1), sf-4(GFPg1), sf-5(GFPg1), and sf-6(GFPg1); having 0, 2, 3, 4, 5, and 6 bases long flap structures, respectively), and an aptamer, in which a complementary sequence of the flap sequence was added to the antisense sequence of the neck region, (sf-ds) were prepared on the basis of s21-sf, and examined for their inhibitory activities. Double-stranded decoy (double-strand of decoy-s and decoy-as) having the same guide sequence and PAM sequence as those of target DNA was used as a control. The sequences of sf-2(GFPg1), sf-3(GFPg1), sf-4(GFPg1), sf-5(GFPg1), sf-6(GFPg1), decoy-s, and decoy-as are shown in Table 20.

TABLE 20

| Aptamer | Nucleotide sequence |
|---|---|
| sf-2(GFPg1) | CGG TGTGAGAGCCTC GAAA GAGGCTCTCACA TCG |
| | CG (SEQ ID NO: 148) |
| sf-3(GFPg1 | CGG TGTGAGAGCCTC GAAA GAGGCTCTCACA TCG |
| | CGC (SEQ ID NO: 149) |
| sf-4(GFPg1) | CGG TGTGAGAGCCTC GAAA GAGGCTCTCACA TCG |
| | CGCC (the same sequence as in s21-sf) |
| | (SEQ ID NO: 131) |
| sf-5(GFPg1) | CGG TGTGAGAGCCTC GAAA GAGGCTCTCACA TCG |
| | CGCCG (SEQ ID NO: 150) |
| sf-6(GFPg1) | CGG TGTGAGAGCCTC GAAA GAGGCTCTCACA TCG |
| | CGCCGA (SEQ ID NO: 151) |
| sf-ds | GGCG CGG TGTGAGAGCCTC GAAA GAGGCTCTCAC |
| | A TCG CGCC (SEQ ID NO: 152) |
| decoy-s | GAGGCTCTCACATCGCGCCGAGGTGAAGTTCGAGGCC |
| | (SEQ ID NO: 152) |
| decoy-as | GGCCTCGAACTTCACCTCGGCGCGATGTGAGAGCCTC |
| | (SEQ ID NO: 153) |

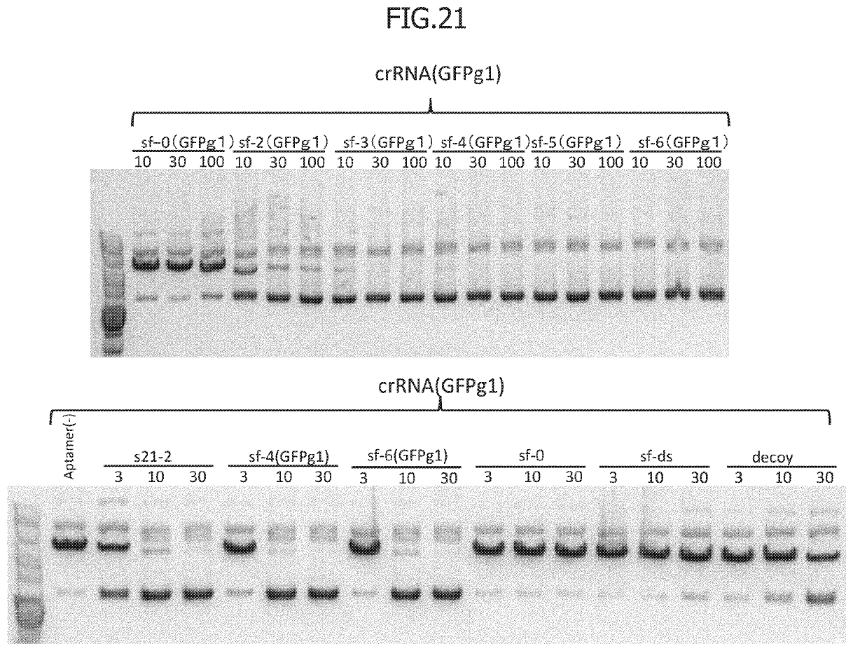

The results are shown in FIG. 21. The sf-0 (flap length: 0) hardly exhibited inhibitory activity even at 100 nM, whereas the inhibitory activity of the aptamers was confirmed to be elevated as the length of the flap structure was increased to the lengths of 2, 3, and 4 bases (sf-2(GFPg1), sf-3(GFPg1), and sf-4(GFPg1)). All the aptamers (sf-4(GFPg1), sf-5(GFPg1), and sf-6(GFPg1)), having the flap structure 4 bases long or longer, exhibited high inhibitory activity. On the other hand, sf-ds having a double-stranded flap sequence moiety, or double-stranded decoy merely exhibited weak inhibitory activity as compared with the stem-flap-type aptamers. These results suggested that the single-stranded flap moiety in the stem-flap-type aptamer is important for the inhibitory activity.

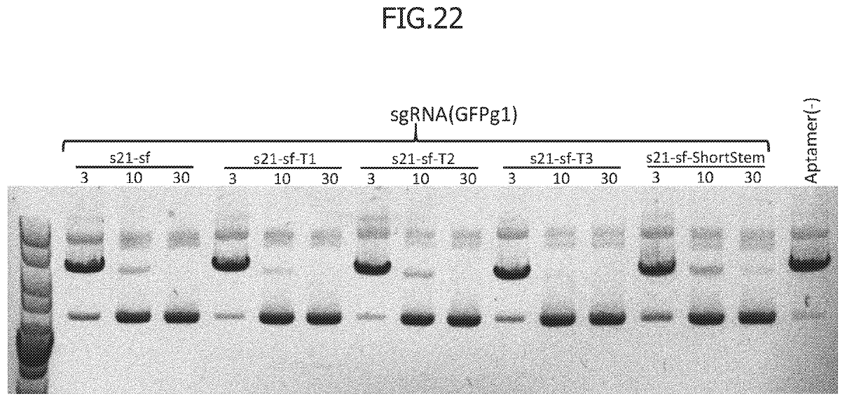

Furthermore, aptamers in which a 1-base to 4-base long sequence forming no double-strand with the flap moiety was added to the antisense sequence of the neck region (s21-sf-T1, s21-sf-T2, and s21-sf-T3) were also evaluated for their inhibitory activities. As a result, all the aptamers had high inhibitory activity (FIG. 22). An aptamer with the structure stabilization region of s21-sf truncated to be 6 base pairs long (s21-sf-ShortStem) exhibited inhibitory activity comparable to that of s21-sf (structure stabilization region: 12 base pairs long) (FIG. 22). From these results, it was concluded that the structure of the structure stabilization region does not seem to influence the inhibitory activity as long as the structure stabilization region is at least 6 base pairs long. The sequences of s21-sf-T1, s21-sf-T2, s21-sf-T3, and s21-sf-ShortStem are as described below (Table 21).

TABLE 21

| Aptamer | Nucleotide sequence |
|---|---|
| s21-sf-T1 | T CGG TGTGAGAGCCTC GAAA GAGGCTCTCACA |
| | TCG CGCC (SEQ ID NO: 154) |
| s21-sf-T2 | TT CGG TGTGAGAGCCTC GAAA GAGGCTCTCACA |
| | TCG CGCC (SEQ ID NO: 155) |
| s21-sf-T3 | TTT CGG TGTGAGAGCCTC GAAA GAGGCTCTCACA |
| | TCG CGCC (SEQ ID NO: 156) |
| s21-sf- | CGG TGTGAG GAAA CTCACA TCG CGCC |
| ShortStem | (SEQ ID NO: 157) |

### Example 9

### Experiment to Verify Inhibitory Activity of Aptamer Against Cas9/crRNA Targeting Another Gene

Stem-flap-type aptamers against Cas9/crRNA(EGFR-b) and Cas9/crRNA(EGFR-c) targeting two sites (EGFR-b and EGFR-c) in an EGF receptor (EGFR) gene sequence were designed (sf(EGFR-b) and sf(EGFR-c)), and examined for their inhibitory activities. The target plasmid used was a plasmid comprising the coding sequence of a fusion protein of human EGF receptor and GFP. The coding sequence of the EGF receptor is as follows.

EGF receptor:
(SEQ ID NO: 159)
ATGCGACCCTCCGGGACGGCCGGGGCAGCGCTCCTGGCGCTGCTGGCTGCG
CTCTGCCCGGCGAGTCGGGCTCTGGAGGAAAAGAAAGTTTGCCAAGGCACG
AGTAACAAGCTCACGCAGTTGGGCACTTTTGAAGATCATTTTCTCAGCCTC
CAGAGGATGTTCAATAACTGTGAGGTGGTCCTTGGGAATTTGGAAATTACC
TATGTGCAGAGGAATTATGATCTTTCCTTCTTAAAGACCATCCAGGAGGTG
GCTGGTTATGTCCTCATTGCCCTCAACACAGTGGAGCGAATTCCTTTGGAA
AACCTGCAGATCATCAGAGGAAATATGTACTACGAAAATTCCTATGCCTTA
GCAGTCTTATCTAACTATGATGCAAATAAAACCGGACTGAAGGAGCTGCCC
ATGAGAAATTTACAGGAAATCCTGCATGGCGCCGTGCGGTTCAGCAACAAC
CCTGCCCTGTGCAACGTGGAGAGCATCCAGTGGCGGGACATAGTCAGCAGT
GACTTTCTCAGCAACATGTCGATGGACTTCCAGAACCACCTGGGCAGCTGC
CAAAAGTGTGATCCAAGCTGTCCCAATGGGAGCTGCTGGGGTGCAGGAGAG
GAGAACTGCCAGAAACTGACCAAAATCATCTGTGCCCAGCAGTGCTCCGGG
CGCTGCCGTGGCAAGTCCCCCAGTGACTGCTGCCACAACCAGTGTGCTGCA
GGCTGCACAGGCCCCCGGGAGAGCGACTGCCTGGTCTGCCGCAAATTCCGA
GACGAAGCCACGTGCAAGGACACCTGCCCCCCACTCATGCTCTACAACCCC
ACCACGTACCAGATGGATGTGAACCCCGAGGGCAAATACAGCTTTGGTGCC
ACCTGCGTGAAGAAGTGTCCCCGTAATTATGTGGTGACAGATCACGGCTCG
TGCGTCCGAGCCTGTGGGGCCGACAGCTATGAGATGGAGGAAGACGGCGTC
CGCAAGTGTAAGAAGTGCGAAGGGCCTTGCCGCAAAGTGTGTAACGGAATA
GGTATTGGTGAATTTAAAGACTCACTCTCCATAAATGCTACGAATATTAAA
CACTTCAAAAACTGCACCTCCATCAGTGGCGATCTCCACATCCTGCCGGTG
GCATTTAGGGGTGACTCCTTCACACATACTCCTCCTCTGGATCCACAGGAA
CTGGATATTCTGAAAACCGTAAAGGAAATCACAGGGTTTTTGCTGATTCAG
GCTTGGCCTGAAAACAGGACGGACCTCCATGCCTTTGAGAACCTAGAAATC
ATACGCGGCAGGACCAAGCAACATGGTCAGTTTTCTCTTGCAGTCGTCAGC
CTGAACATAACATCCTTGGGATTACGCTCCCTCAAGGAGATAAGTGATGGA
GATGTGATAATTTCAGGAAACAAAAATTTGTGCTATGCAAATACAATAAAC
TGGAAAAAACTGTTTGGGACCTCCGGTCAGAAAACCAAAATTATAAGCAAC
AGAGGTGAAAACAGCTGCAAGGCCACAGGCCAGGTCTGCCATGCCTTGTGC
TCCCCCGAGGGCTGCTGGGGCCCGGAGCCCAGGGACTGCGTCTCTTGCCGG
AATGTCAGCCGAGGCAGGGAATGCGTGGACAAGTGCAACCTTCTGGAGGGT
GAGCCAAGGGAGTTTGTGGAGAACTCTGAGTGCATACAGTGCCACCCAGAG
TGCCTGCCTCAGGCCATGAACATCACCTGCACAGGACGGGGACCAGACAAC
TGTATCCAGTGTGCCCACTACATTGACGGCCCCCACTGCGTCAAGACCTGC
CCGGCAGGAGTCATGGGAGAAAACAACACCCTGGTCTGGAAGTACGCAGAC
GCCGGCCATGTGTGCCACCTGTGCCATCCAAACTGCACCTACGGATGCACT
GGGCCAGGTCTTGAAGGCTGTCCAACGAATGGGCCTAAGATCCCGTCCATC
GCCACTGGGATGGTGGGGGCCCTCCTCTTGCTGCTGGTGGTGGCCCTGGGG
ATCGGCCTCTTCATGCGAAGGCGCCACATCGTTCGGAAGCGCACGCTGCGG
AGGCTGCTGCAGGAGAGGGAGCTTGTGGAGCCTCTTACACCCAGTGGAGAA
GCTCCCAACCAAGCTCTCTTGAGGATCTTGAAGGAAACTGAATTCAAAAAG
ATCAAAGTGCTGGGCTCCGGTGCGTTCGGCACGGTGTATAAGGGACTCTGG
ATCCCAGAAGGTGAGAAAGTTAAAATTCCCGTCGCTATCAAGGAATTAAGA
GAAGCAACATCTCCGAAAGCCAACAAGGAAATCCTCGATGAAGCCTACGTG
ATGGCCAGCGTGGACAACCCCCACGTGTGCCGCCTGCTGGGCATCTGCCTC
ACCTCCACCGTGCAACTCATCACGCAGCTCATGCCCTTCGGCTGCCTCCTG
GACTATGTCCGGGAACACAAAGACAATATTGGCTCCCAGTACCTGCTCAAC
TGGTGTGTGCAGATCGCAAAGGGCATGAACTACTTGGAGGACCGTCGCTTG
GTGCACCGCGACCTGGCAGCCAGGAACGTACTGGTGAAAACACCGCAGCAT
GTCAAGATCACAGATTTTGGGCTGGCCAAACTGCTGGGTGCGGAAGAGAAA
GAATACCATGCAGAAGGAGGCAAAGTGCCTATCAAGTGGATGGCATTGGAA
TCAATTTTACACAGAATCTATACCCACCAGAGTGATGTCTGGAGCTACGGG
GTGACCGTTTGGGAGTTGATGACCTTTGGATCCAAGCCATATGACGGAATC
CCTGCCAGCGAGATCTCCTCCATCCTGGAGAAAGGAGAACGCCTCCCTCAG
CCACCCATATGTACCATCGATGTCTACATGATCATGGTCAAGTGCTGGATG
ATAGACGCAGATAGTCGCCCAAAGTTCCGTGAGTTGATCATCGAATTCTCC
AAAATGGCCCGAGACCCCCAGCGCTACCTTGTCATTCAGGGGGATGAAAGA
ATGCATTTGCCAAGTCCTACAGACTCCAACTTCTACCGTGCCCTGATGGAT
GAAGAAGACATGGACGACGTGGTGGATGCCGACGAGTACCTCATCCCACAG
CAGGGCTTCTTCAGCAGCCCCTCCACGTCACGGACTCCCCTCCTGAGCTCT
CTGAGTGCAACCAGCAACAATTCCACCGTGGCTTGCATTGATAGAAATGGG
CTGCAAAGCTGTCCCATCAAGGAAGACAGCTTCTTGCAGCGATACAGCTCA
GACCCCACAGGCGCCTTGACTGAGGACAGCATAGACGACACCTTCCTCCCA
GTGCCTGAATACATAAACCAGTCCGTTCCCAAAAGGCCCGCTGGCTCTGTG
CAGAATCCTGTCTATCACAATCAGCCTCTGAACCCCGCGCCCAGCAGAGAC
CCACACTACCAGGACCCCCACAGCACTGCAGTGGGCAACCCCGAGTATCTC
AACACTGTCCAGCCCACCTGTGTCAACAGCACATTCGACAGCCCTGCCCAC
TGGGCCCAGAAAGGCAGCCACCAAATTAGCCTGGACAACCCTGACTACCAG
CAGGACTTCTTTCCCAAGGAAGCCAAGCCAAATGGCATCTTTAAGGGCTCC
ACAGCTGAAAATGCAGAATACCTAAGGGTCGCGCCACAAAGCAGTGAATTT
ATTGGAGCA

The sequences of crRNAs targeting two sites (EGFR-b and EGFR-c) in the EGF receptor (EGFR) gene sequence, and the sequences of the stem-flap-type aptamers sf(EGFR-b) and sf(EGFR-c) are as shown below (Table 22).

TABLE 22

| | Nucleotide sequence |
|---|---|
| crRNA | |
| crRNA(EGFR-b) | aucauaauuccucugcacau guuuuagagcuaug |
| | cuguuug (SEQ ID NO: 160) |
| crRNA(EGFR-c) | ccacuguguugagggcaaug guuuuagagcuaug |
| | cuguuug (SEQ ID NO: 161) |
| Aptamer | |
| sf(EGFR-b) | CGG TGTGAGAGCCTC GAAA GAGGCTCTCACA |
| | TCG ATGT (SEQ ID NO: 162) |
| sf(EGFR-c) | CGG TGTGAGAGCCTC GAAA GAGGCTCTCACA |
| | TCG CATT (SEQ ID NO: 163) |

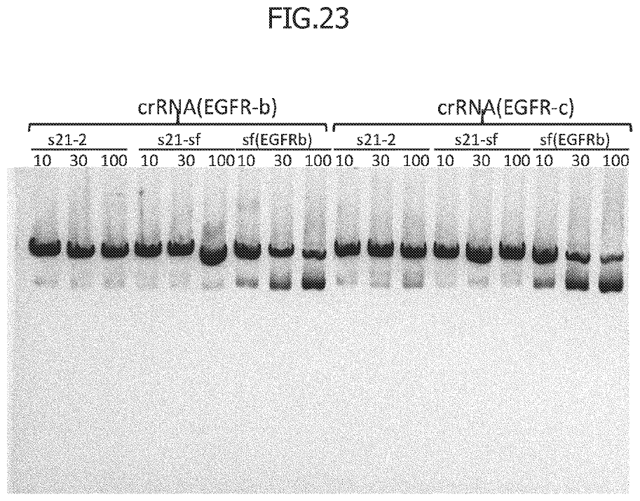

The results are shown in FIG. 23. The stem-flap-type aptamers sf(EGFR-b) and sf(EGFR-c) against Cas9/crRNA(EGFR-b) and Cas9/crRNA(EGFR-c) produced slightly lower inhibitory activities than those of the stem-flap-type aptamers against Cas9/crRNA targeting GFP, analyzed in the preceding Examples. This is presumably because the double-strand-forming sequences of crRNA and each aptamer were AT-rich, and a double-strand was difficult to form therebetween. Accordingly, in order to facilitate forming a double strand, aptamers with the flap structure having a length extended to be 5 bases long or 6 bases long were designed (sf5(EGFR-b), sf5(EGFR-c), sf5(EGFR-c), and sf6(EGFR-c)), and evaluated again for their inhibitory activities. Antisense DNA (As(EGFR-b) and As(EGFR-c)) was used as a control. These sequences are shown in Table 23.

TABLE 23

| | Nucleotide sequence |
|---|---|
| Aptamer | |
| sf5(EGFR-b) | CGG TGTGAGAGCCTC GAAA GAGGCTCTCACA TCG |
| | ATGTG (SEQ ID NO: 164) |
| sf6(EGFR-b) | CGG TGTGAGAGCCTC GAAA GAGGCTCTCACA TCG |
| | ATGTGC (SEQ ID NO: 165) |
| sf5(EGFR-c) | CGG TGTGAGAGCCTC GAAA GAGGCTCTCACA TCG |
| | CATTG (SEQ ID NO: 166) |
| sf6(EGFR-c) | CGG TGTGAGAGCCTC GAAA GAGGCTCTCACA TCG |
| | CATTGC (SEQ ID NO: 167) |
| Antisense | |
| DNA | |
| As(EGFR-b) | ATGTGCAGAGGAATTATGAT (SEQ ID NO: 168) |
| As(EGFR-c) | CATTGCCCTCAACACAGTGG (SEQ ID NO: 169) |

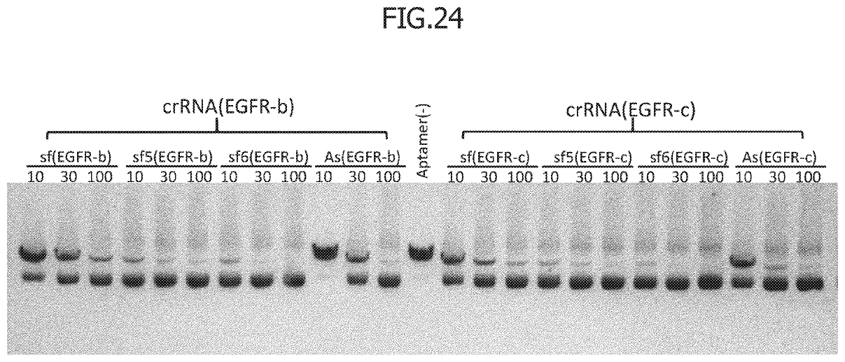

The results are shown in FIG. 24. As expected, the extension of the flap length recovered inhibitory activity against Cas9/crRNA for both the target sites in EGFR. The stem-flap-type aptamers sf6(EGFR-b) and sf6(EGFR-c), having a flap structure 6 bases long, exhibited inhibitory activity equivalent to that of the stem-flap-type aptamers against Cas9/crRNA targeting GFP.

Furthermore, a stem-flap-type aptamer against Cas9/crRNA(EpCAM) targeting EpCAM gene was designed (sf(EpCAM)), and examined for its inhibitory activity. The target plasmid used was a plasmid into which a target sequence in the EpCAM gene was cloned. The sequence is shown below.

EpCAM:
(SEQ ID NO: 170)
GTGCACCAACTGAAGTACACCGG

TABLE 24

| | Nucleotide sequence |
|---|---|
| crRNA | |
| crRNA(EpCAM) | gugcaccaacugaaguacac guuuuagagcuaugcu |
| | guuug (SEQ ID NO: 171) |
| Aptamer | |
| sf(EpCAM) | CGG TGTGAGAGCCTC GAAA GAGGCTCTCACA |
| | TCG GTGT (SEQ ID NO: 172) |

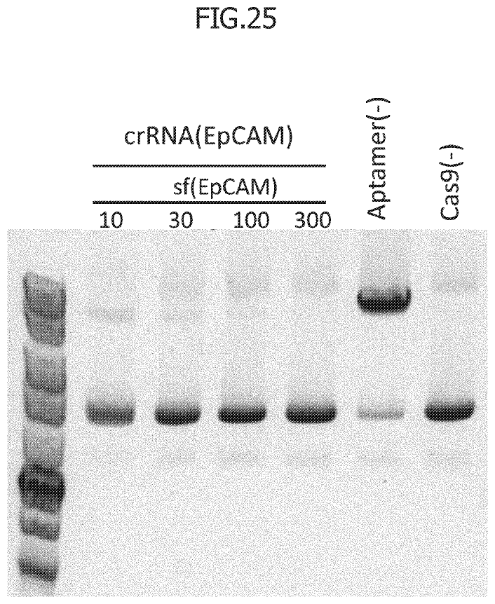

The results are shown in FIG. 25. The stem-flap-type aptamer (sf(EpCAM)) against Cas9/crRNA(EpCAM) also exhibited high inhibitory activity, as in the stem-flap-type aptamers against Cas9/crRNA targeting other genes.

### Example 10

### Design of Aptamer Against Cpfl (Cas9 Homolog) and Experiment to Verify Inhibitory Activity

Various homologs of Cas9 exist and are known to recognize respective PAMs including different sequences. Accordingly, an experiment was conducted to verify whether the method for designing the Cas9-inhibiting aptamers studied in Examples described above could be applied to a Cas9 homolog recognizing another PAM sequence.

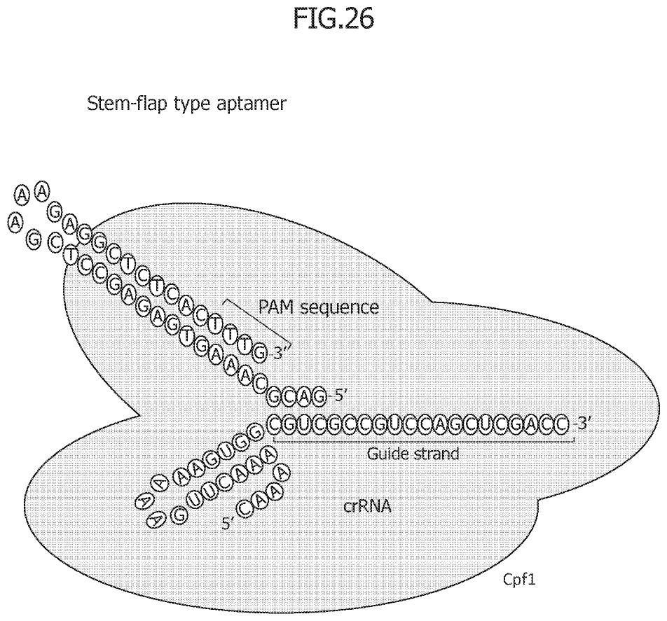

Here, Cpf1, a CRISPR genome editing enzyme most commonly used next to Cas9, was used as the Cas9 homolog. Cpf1 recognizes a PAM sequence (TTTN) upstream of (i.e., 5′-terminally adjacent to) the guide RNA-targeted sequence in a target gene, and cleaves the double-stranded DNA on the target gene. Hence, for stem-flap-type aptamers against Cpf1/crRNA, the flap structure moiety should need to be placed at an end opposite (i.e., the 5′ end) to that for stem-flap-type aptamers against Cas9/crRNA. Accordingly, a stem-flap-type aptamer targeting Cpf1/crRNA (sf-c(GFPa)) as shown in FIG. 26 and crRNA(GFPa) targeting GFP were designed, and the inhibitory activity of the aptamer was evaluated. Since a G:T base pair in the neck region of the Cas9-inhibiting aptamers contributed to increase in inhibitory activity, three types of stem-flap-type aptamers having a T:G base pair similarly introduced therein were also prepared (sf-c1(GFPa), sf-c2(GFPa), and sf-c3(GFPa)). The plasmid comprising GFP gene was the same as that used in Example 2. The Cpf1 enzyme and the crRNA used were purchased from Integrated Device Technology, Inc. (IDT).

The sequences of crRNA(GFPa) and the aptamers are shown in Table 25. In the table, sequences considered to form a double strand with crRNA are underlined.

TABLE 25

| | Nucleotide sequence |
|---|---|
| crRNA | (only guide sequence described) |
| crRNA(GFPa) | cgucgccguccagcucgacc |
| | (SEQ ID NO: 173) |
| Aptamer | Nucleotide sequence |
| sf-c(GFPa) | GACG CAAA GTGAGAGCCTC GAAA GAGGCTCTCAC |
| | TTTG (SEQ ID NO: 174) |
| sf-c1(GFPa) | GACG CGAA GTGAGAGCCTC GAAA GAGGCTCTCAC |
| | TTTG (SEQ ID NO: 175) |
| sf-c2(GFPa) | GACG CAGA GTGAGAGCCTC GAAA GAGGCTCTCAC |
| | TTTG (SEQ ID NO: 176) |
| sf-c3(GFPa) | GACG CAAG GTGAGAGCCTC GAAA GAGGCTCTCAC |
| | TTG( SEQ ID NO: 177) |

Cpf1 (final concentration: 60 nM) and crRNA(GFPa) (final concentration: 50 nM) were mixed in a buffer (50 mM Tris/HCl pH 7.9, 100 mM NaCl, 10 mM MgCl₂, and 100 ug/ml BSA), and left standing for 20 minutes. Then, the plasmid and each aptamer (final concentration: 3 nM) were added thereto, and incubated at 37° C. for 10 minutes. Then, the cleavage of the plasmid was observed by 0.65% agarose gel electrophoresis.

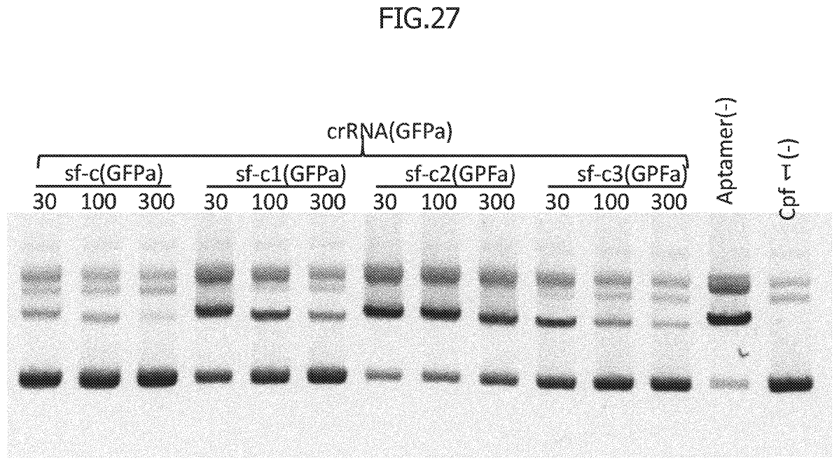

The results are shown in FIG. 27. The stem-flap-type aptamers designed for Cpf1/crRNA also exhibited high inhibitory activity, as in the stem-flap-type aptamers against Cas9/crRNA. All the aptamers having a T:G mismatch inserted in the neck region exhibited decrease in inhibitory activity.

In order to verify the generality of the design of the stem-flap-type aptamers against Cpf1/crRNA, crRNAs targeting three target sequences in EGFR (EGFR-1, EGFR-2, and EGFR-3) and stem-flap-type aptamers respectively compatible therewith (sf-c(EGFR-1), sf-c(EGFR-2), and sf-c(EGFR-3)) were designed, and the inhibitory activity of the aptamers was examined. The three target sequences in EGFR and the sequences of the stem-flap-type aptamers respectively compatible therewith are shown in Table 26. In the table, sequences considered to form a double strand with crRNA are underlined.

TABLE 26

| | Nucleotide sequence |
|---|---|
| crRNA | (only guide sequence described) |
| crRNA(EGFR-1) | gugccaccugcgugaagaag (SEQ ID NO: 178) |
| crRNA(EGFR-2) | cgccagaccaggcagucgcu (SEQ ID NO: 179) |
| crRNA(EGFR-3) | uggcaguucuccucuccugc (SEQ ID NO: 180) |
| Aptamer | Nucleotide sequence |
| sf-c(EGFR-1) | GCAC CAA AGTGAGAGCCTC GAAA GAGGCTCT |
| | CACT TTG (SEQ ID NO: 181) |
| sf-c(EGFR-2) | GCCG CAA AGTGAGAGCCTC GAAA GAGGCTCT |
| | CACT TTG (SEQ ID NO: 182) |
| sf-c(EGFR-3) | GCCA CAA AGTGAGAGCCTC GAAA GAGGCTCT |
| | CACT TTG (SEQ ID NO: 183) |

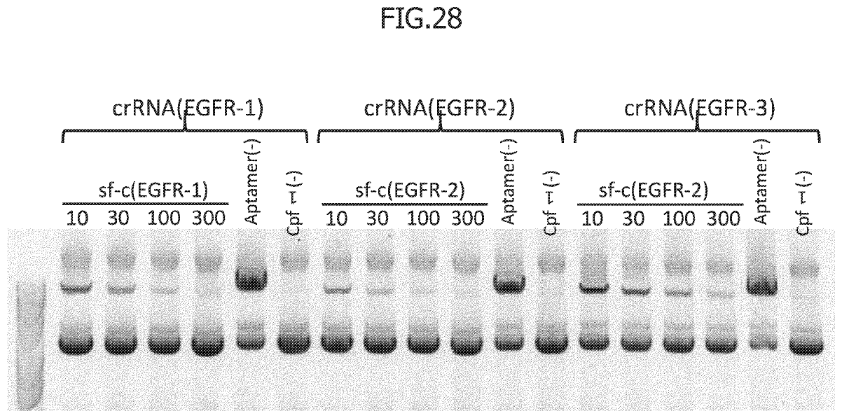

The results are shown in FIG. 28. All the stem-flap-type aptamers against Cpf1/crRNA exhibited high inhibitory activities.

### Example 11

### Experiment to Verify Intracellular Inhibitory Activity of Aptamer

In order to confirm whether the aptamer of the present invention could also inhibit Cas9/crRNA intracellularly, a reporter cell line expressing a fluorescent protein (mScarlet) resulting from genome editing was prepared (293-mScarlet). The 293-mScarlet cells were prepared by inserting a reporter cassette in which a target sequence having a stop codon was inserted upstream of the coding sequence of mScarlet, into the genome of HEK293 using the Flp-In system from Thermo Fisher Scientific Inc. Before genome editing, the 293-mScarlet cells does not express mScarlet due to the presence of the stop codon upstream of the coding sequence of mScarlet. However, when genome editing dependent on the target sequence occurs, the stop codon is eliminated by base insertion or deletion at the cleaved target site. If the cleaved site is correctly repaired in frame with the coding sequence of mScarlet, the mScarlet fluorescent protein is expressed.

The sequence of the reporter cassette is shown below. The lower-case characters represent the sequence of mScarlet, and the upper-case characters represent the target sequence containing a stop codon. A target sequence of Cas9/crRNA is underlined. TAG positioned at the 3′ end of the target sequence is a stop codon.

Reporter cassette (target sequence-mScarlet):
(SEQ ID NO: 184)
ATGGCGTCTTCTTCTCATTTCACACCGAAGCAGAGTTTTTCGGATTCCCGA
GTAGCAGATGACCATGACAAGTAGCGGCAGGACCAGCCCCAAGATGACTTG
GAGATAACTACTAAGAAGAAAACGGTGAGCAAGGGCGAGGAGgtgagcaag
ggcgaggcagtgatcaaggagttcatgcggttcaaggtgcacatggagggc
tccatgaacggccacgagttcgagatcgagggcgagggcgagggccgcccc
tacgagggcacccagaccgccaagctgaaggtgaccaagggtggccccctg
cccttctcctgggacatcctgtcccctcagttcatgtacggctccagggcc
ttcatcaagcaccccgccgacatccccgactactataagcagtccttcccc
gagggcttcaagtgggagcgcgtgatgaacttcgaggacggcggcgccgtg
accgtgacccaggacacctccctggaggacggcaccctgatctacaaggtg
aagctccgcggcaccaacttccctcctgacggccccgtaatgcagaagaag
acaatgggctgggaagcgtccaccgagcggttgtaccccgaggacggcgtg
ctgaagggcgacattaagatggccctgcgcctgaaggacggcggccgctac
ctggcggacttcaagaccacctacaaggccaagaagcccgtgcagatgccc
ggcgcctacaacgtcgaccgcaagttggacatcacctcccacaacgaggac
tacaccgtggtggaacagtacgaacgctccgagggccgccactccaccggc
ggcatggacgagctgtacaag

A Cas9 protein (2 μg)/crRNA:tracrRNA (0.5 μg) complex was introduced into the 293-mScarlet cell line placed in a 24-well plate using Lipofectamine™ CRISPRMAX™ Cas9 Transfection Reagent (Thermo Fisher Scientific Inc.). At the same time with (i.e., 0 hours after) or 6 hours after the introduction of Cas9/crRNA:tracrRNA, an aptamer (sf-Sca) (final concentration: 100 nM) or a negative control aptamer (ds-cont) (final concentration: 100 nM) were introduced thereto. Then, the cells were cultured for 3 days. The presence or absence of mScarlet expression was observed under a microscope. The introduction of the aptamers 6 hours after the introduction of Cas9/crRNA:tracrRNA was performed using Lipofectamine 2000 (Thermo Fisher Scientific Inc.). The sequences of the crRNA and the aptamers used are shown in Table 27. Thiolated bases are indicated by \hat{ }.

TABLE 27

| | Nucleotide sequence |
|---|---|
| crRNA | |
| crRNA(Sca) | cagaugaccaugacaaguag guuuuagagcuaugcug |
| | uuug (SEQ ID NO: 185) |
| Aptamer | |
| sf-Sca | C\hat{ }G\hat{ }G TGTGAGAGCCTC GAAA GAGGCTCTCACA |
| | TCG CTAC\hat{ }T\hat{ }T (SEQ ID NO: 186) |
| ds-cont | G\hat{ }A\hat{ }GGCTCTCACA GAAA TGTGAGAGCC\hat{ }T\hat{ }C |
| | (SEQ ID NO: 187) |

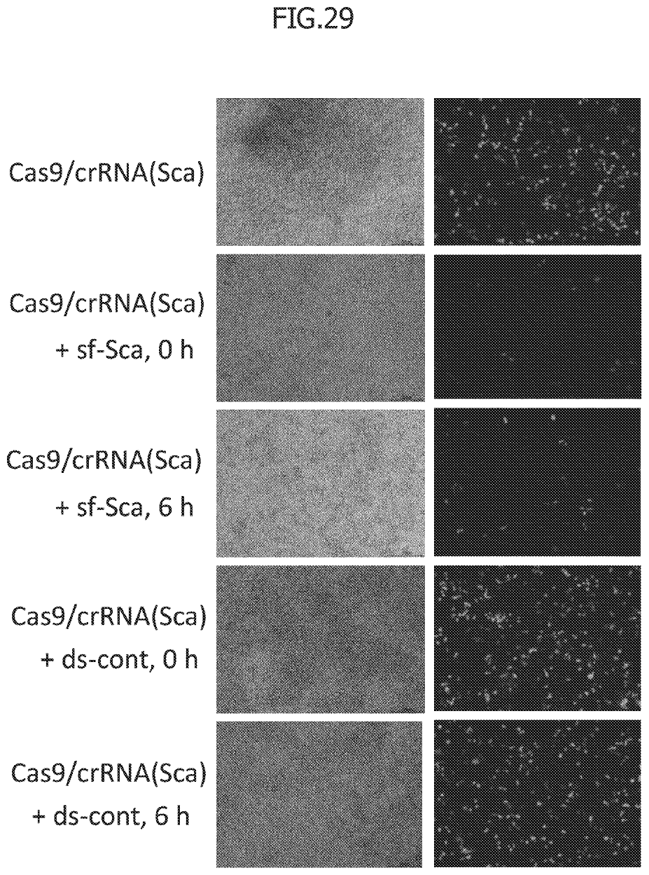

The results are shown in FIG. 29. The aptamer sf-Sca both when introduced at the same time with the introduction of Cas9/crRNA:tracrRNA (Cas9/crRNA(Sca)+sf-Sca, 0 h) and when introduced 6 hours thereafter (Cas9/crRNA(Sca)+sf-Sca, 6 h) markedly inhibited genome editing in the cells.

### Example 12

### Experiment on Intracellular Inhibition of Genome Editing by Aptamer Comprising LNA

In order to further improve the intracellular inhibitory effect of the aptamer on genome editing, an aptamer with the flap structure moiety substituted with LNA was synthesized (sf-Sca-LNA), and evaluated for its inhibitory activity using the same experimental system and procedure as those of Example 11. The aptamer (final concentration: 100 nM) was introduced into cells at the same time with the introduction of Cas9/sgRNA. Then, the cells were cultured for 3 days. The presence or absence of mScarlet expression was observed under a microscope. In order to quantitatively evaluate the inhibitory activity of the aptamer, the cells thus observed were recovered, and the number of cells in which genome editing occurred (cells expressing mScarlet) was counted using FACS. The sequence of sf-Sca-LNA is shown in Table 28. LNA is underlined (except that C in LNA is 5-methylcytosine). Thiolated bases are indicated by \hat{ }.

TABLE 28

| Aptamer | Nucleotide sequence |
|---|---|
| sf-Sca-LNA | C\hat{ }G\hat{ }G TGTGAGAGCCTC GAAA GAGGCTCTCACA TCG |
| | CTAC\hat{ }T\hat{ }T (SEQ ID NO: 188) |

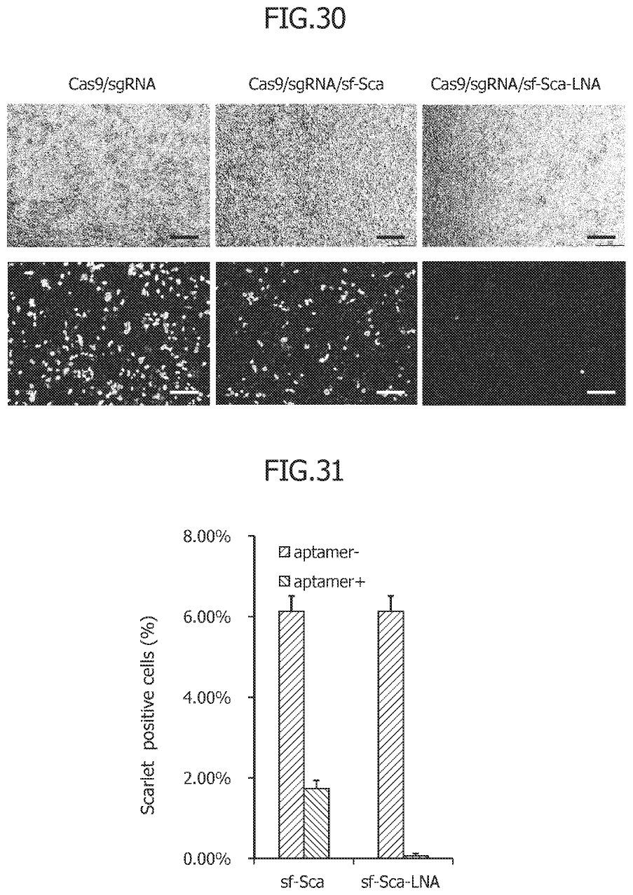

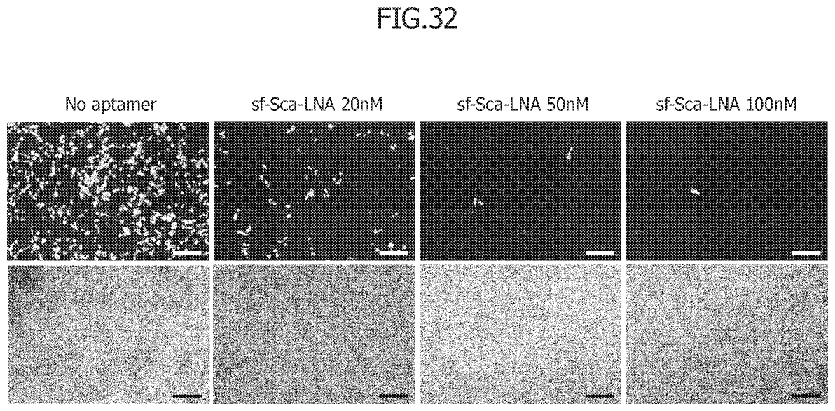

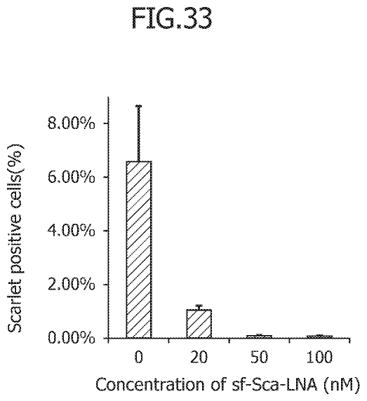

The results are shown in FIGS. 30 and 31. The DNA aptamer (sf-Sca) intracellularly inhibited genome editing by approximately 60 to 70%, whereas the LNA/DNA aptamer (sf-Sca-LNA) intracellularly inhibited genome editing almost completely. Results of examining the concentration dependence of sf-Sca-LNA are shown in FIGS. 32 and 33. These results demonstrated that sf-Sca-LNA can inhibit genome editing almost completely at final concentrations of 50 nM or higher.

### Example 13

### Experiment on Inhibition of Genome Editing of Endogenous Gene by LNA-Modified Aptamer

Next, examination was made on whether each aptamer could inhibit the Cas9-mediated genome editing of an endogenous gene. HPRT1 and EMX1 were selected as targeted endogenous genes. The crRNA targeting each of the genes was synthesized, and Cas9/crRNA:tracrRNA complexes were prepared. Each of the obtained complexes was introduced into 293FT cells (Thermo Fisher Scientific Inc.) at the same time with the introduction of the aptamer by the same procedure as that in Example 11. The cells were cultured for 3 days. Then, the cells were collected. DNA was extracted from the cells using Guide-it Mutation Detection kit (Takara Bio Inc.). A target site of genome editing was amplified by PCR. The PCR product was treated with T7E1 enzyme and then electrophoresed on 2% agarose gel to detect indel (insertion/deletion) resulting from genome editing. The sequences of the crRNAs, the aptamers, and the primers used are shown in Table 29. In the table, the upper-case characters represent DNA, and the lower-case characters represent RNA. LNA is underlined (except that C in LNA is 5-methylcytosine). Thiolated bases are indicated by \hat{ }.

TABLE 29

| | Nucleotide sequence |
|---|---|
| crRNA | |
| crRNA(HPRT1) | gcauuucucaguccuaaaca guuuuagagcuaugcu |
| | guuug (SEQ ID NO: 189) |
| crRNA(EMX1) | gaguccgagcagaagaagaa guuuuagagcuaugcu |
| | guuug (SEQ ID NO: 190) |
| Aptamer | |
| sf-HPRT1 | C\hat{ }G\hat{ }G TGTGAGAGCCTC GAAA GAGGCTCTCACA |
| | TCG TGTT\hat{ }T\hat{ }A (SEQ ID NO: 191) |
| sf-EMX1 | C\hat{ }G\hat{ }G TGTGAGAGCCTC GAAA GAGGCTCTCACA |
| | TCG TTCT\hat{ }T\hat{ }C (SEQ ID NO: 192) |
| Primer | |
| HPRT1-F | ACA TCA GCA GCT GTT CTG |
| | (SEQ ID NO: 193) |
| HPRT1-R | GGC TGA AAG GAG AGA ACT |
| | (SEQ ID NO: 194) |
| EMX1-F | GCC ATC CCC TTC TGT GAA TGT TAG AC |
| | (SEQ ID NO: 195) |
| EMX1-R | CGG AAT CTA CCA CCC CAG GCT CT |
| | (SEQ ID NO: 196) |

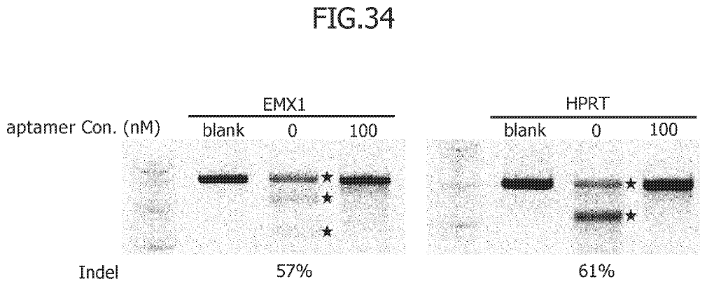

The results are shown in FIG. 34. The aptamers (sf-HPRT1 and sf-EMX1) designed by the method of the present invention were confirmed to be able to inhibit genome editing almost completely both for HPRT1 and for EMX1.

1. A nucleic acid aptamer inhibiting binding activity or enzymatic activity of a complex comprising guide RNA and nuclease against a target nucleic acid serving as a substrate of the complex, the nucleic acid aptamer comprising the following regions (1) to (3):
(1) a single-stranded guide RNA recognition region comprising a guide RNA-recognizing oligonucleotide including a sequence recognizing the guide RNA;
(2) a double-stranded neck region comprising a PAM sequence corresponding to the nuclease in one strand, the neck region comprising a first neck oligonucleotide including the PAM sequence, and a second neck oligonucleotide including a sequence having complementarity to the PAM sequence; and
(3) a double-stranded structure stabilization region comprising a first structure-stabilizing oligonucleotide and a second structure-stabilizing oligonucleotide,
wherein the region (1) is linked to the second neck oligonucleotide to form a flap structure, or the region (1) is linked to the first neck oligonucleotide and the second neck oligonucleotide to form a loop structure in which the guide RNA-recognizing oligonucleotide in the region (1) is adjacent to the second neck oligonucleotide, and
wherein the region (2) and the region (3) are linked to each other to together form a stem structure.

2. The nucleic acid aptamer according to claim 1, wherein the nuclease is a nuclease of the CRISPR-Cas family.

3. The nucleic acid aptamer according to claim 1, wherein the guide RNA-recognizing oligonucleotide in the region (1) includes a 2-base to 30-base long sequence adjacent to the PAM sequence of the target nucleic acid.

4. The nucleic acid aptamer according to claim 3, wherein the guide RNA-recognizing oligonucleotide in the region (1) includes a 3-base to 22-base long sequence adjacent to the PAM sequence of the target nucleic acid.

5. The nucleic acid aptamer according to claim 1, wherein the region (1) is 6 to 50 bases long.

6. The nucleic acid aptamer according to claim 1, wherein the region (1) comprises a bridged nucleic acid.

7. The nucleic acid aptamer according to claim 1, wherein the region (2) comprises a mismatch or a bulge.

8. The nucleic acid aptamer according to claim 1, wherein the first neck oligonucleotide in the region (2) is 5′-NGG-3′, and the complex is CRISPR-Cas9.

9. The nucleic acid aptamer according to claim 1, wherein the first neck oligonucleotide in the region (2) is 5′-TTTN-3′, and the complex is CRISPR-Cpf1.

10. The nucleic acid aptamer according to claim 1, wherein the region (3) is at least 4 base pairs long.

11. The nucleic acid aptamer according to claim 1, wherein the nucleic acid aptamer comprises a phosphorothioate modification.

12. A method for producing a nucleic acid aptamer inhibiting binding activity or enzymatic activity of a complex comprising guide RNA and nuclease against a target nucleic acid served as a substrate of the complex, the method comprising the steps of:
(1) determining a guide RNA-recognizing oligonucleotide including a sequence recognizing the guide RNA;
(2) determining a first neck oligonucleotide including a PAM sequence compatible with the nuclease, and a second neck oligonucleotide including a sequence having complementarity to the first neck oligonucleotide;
(3) determining a first structure-stabilizing oligonucleotide and a second structure-stabilizing oligonucleotide;
(4) adding the first structure-stabilizing oligonucleotide to the first neck oligonucleotide;
(5) adding the second structure-stabilizing oligonucleotide to the second neck oligonucleotide;
(6) linking the guide RNA-recognizing oligonucleotide to the second neck oligonucleotide; and
(7) synthesizing a nucleic acid comprising the sequences designed by the steps (1) to (6).

13. The method according to claim 12, further comprising the step of:
(5′) linking the first structure-stabilizing oligonucleotide to the second structure-stabilizing oligonucleotide.

14. The method according to claim 12, further comprising the step of:
(6′) linking the guide RNA-recognizing oligonucleotide to the first neck oligonucleotide either directly or via a linker oligonucleotide.

15. A method for inhibiting binding activity or enzymatic activity of a complex comprising guide RNA and nuclease against a target nucleic acid served as a substrate of the complex, the method comprising the steps of:
(1) preparing a reaction solution containing the complex and the target nucleic acid; and
(2) adding the nucleic acid aptamer according to claim 1 to the reaction solution.

16. A method for intracellularly inhibiting binding activity or enzymatic activity of a complex comprising guide RNA and nuclease against a target nucleic acid served as a substrate of the complex, the method comprising the steps of:
(1) introducing the complex to a cell containing the target nucleic acid; and
(2) introducing the nucleic acid aptamer according to claim 1 to the cell.

17. A genome editing method comprising the steps of:
(1) introducing a complex comprising guide RNA and nuclease of the CRISPR-Cas family to a cell; and
(2) introducing the nucleic acid aptamer according to claim 1 to the cell.
